(19) United States
(12) Patent Application Publication
(10) Pub. No.: US 2019/0054117 A1
(43) Pub. Date: Feb. 21, 2019

(54) DIMERIZATION SWITCHES AND USES THEREOF

(71) Applicants: Andreas Loew, Somerville, MA (US); Brian Edward Vash, Cambridge, MA (US)

(72) Inventors: Andreas Loew, Somerville, MA (US); Brian Edward Vash, Cambridge, MA (US)

(73) Assignee: Novartis AG, Basel (CH)

(21) Appl. No.: 15/536,790

(22) PCT Filed: Dec. 18, 2015
(86) PCT No.: PCT/IB2015/059796

§ 371 Date: Jun. 16, 2017

Related U.S. Application Data
(60) Provisional application No. 62/094,427, filed on Dec. 19, 2014.

(51) Int. Cl. A61K 35/17; C12N 15/62; C12N 9/12; C12N 9/90; A61K 48/00

(52) U.S. Cl. CPC A61K 35/17; C12N 15/62; C12N 9/12; C12N 9/90; A61K 48/0066; C12Y 207/11001; C12N 5/0636; C07K 2319/09; C07K 2319/70; C07K 2319/81; C07K 2319/02; C07K 2319/03; C12Y 502/01008

(57) ABSTRACT

The present invention provides gene editing systems comprising gene editing dimerization switches comprising a first and second gene editing switch domain that allow for the regulation of a gene editing function by the introduction, e.g., administration, of a gene editing dimerization molecule having the ability to bring together a first gene editing switch domain and a second gene editing switch domain. A regulated gene editing function provides, e.g., less off-target side effects, and increases the therapeutic window. The present invention also provides improved FKBP/FRB-based dimerization switches wherein the FRB switch domain or the FKBP switch domain, or both the FRB and FKBP switch domains, comprise one or more mutations that optimize performance, e.g., that alter, e.g., enhance the formation of a complex between the first switch domain, the second switch domain, and the dimerization molecule, rapamycin, or a rapalog, e.g., RAD001.

### RELATED APPLICATIONS

This application is a national phase entry under 35 USC § 371 of PCT Application No. PCT/M2015/059796, filed on Dec. 18, 2015, which claims priority under 35 USC § 119 to U.S. Provisional Application No. 62/094,427, filed Dec. 19, 2014, all of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 4, 2016, is named PAT056609-WO-PCT_SL.txt and is 102,400 bytes in size.

### BACKGROUND

Dimerization switches containing FK506 binding protein (FKBP)-derived domains and FRB (derived from FKBP rapamycin binding protein, also known as mTOR) domains have been described. Such dimerization switches rely upon dimerization of the FKBP and FRB domains, which results in the coupling of the fused protein components to trigger a desired biological event (Spencer et al., 1993, Science 262:1019-1024). Rapamycin and derivatives thereof (also known as rapalogs) are capable of dimerizing the FKBP/FRB switch domains. However, rapamycin and many rapalogs have potent immunosuppressive activity, which limit the use of such biological switches in certain therapeutic and in vivo applications. Thus, there is a need for improved dimerization switches that allow the use of a wider dosage range of rapamycin or rapalogs that does not induce immunosuppression or other adverse effects in vivo.

Recently, gene editing systems such as zinc finger nucleases, CRISPR/Cas systems, transcription activator-like effector nucleases (TALENs) and meganucleases have emerged as tools for the regulation of genes. However, therapeutic use, especially in vivo use, of these systems is limited by, among other things, uncontrolled activity and off-target gene editing. Thus, there is a need for regulatable gene editing systems.

### SUMMARY

The present invention features a dimerization switch which comprises:

(a) a polypeptide comprising a first switch domain comprising an FRB fragment or analog thereof, e.g., of SEQ ID NO:2, having the ability to form a complex between the FRB fragment or analog thereof, a FKBP fragment or analog thereof and a dimerization molecule; and

(b) a polypeptide comprising second switch domain comprising an FKBP fragment or analog thereof, e.g., of SEQ ID NO:1 or 3, having the ability to form a complex between the FKBP fragment or analog thereof, a FRB fragment or analog thereof and a dimerization molecule.

In some aspects the dimerization switch comprises one or more of the switch domains 1) to 10), below:
1) In an aspect, the first switch domain comprises one or more mutations each of which enhances formation of a complex between a first switch domain, a second switch domain (e.g., a FKBP derived switch domain), and a dimerization molecule (e.g., a rapamycin, or a rapalog, e.g., RAD001). In an aspect, the enhancement is additive or more than additive.
2) In an aspect, the first switch domain comprises a mutation at E2032, e.g., E2032I or E2032L, and at T2098, e.g., T2098L.
3) In an aspect, the first switch domain comprises the mutation E2032I, and further comprises a mutation at one or a plurality of L2031, S2035, R2036, F2039, G2040, T2098, W2101, D2102, Y2105, or F2108.
4) In an aspect, the first switch domain comprises a mutation at E2032I and at T2098. In one aspect the mutation at T2098 is T2098L.
5) In an aspect, the first switch domain comprises the mutation at E2032L, and further comprises a mutation at one or more of L2031, S2035, R2036, F2039, G2040, T2098, W2101, D2102, Y2105, or F2108.
6) In an aspect, the first switch domain comprises a mutation at E2032L and at T2098. In one aspect the mutation at T2098 is T2098L.
7) In an aspect, the first switch domain comprises a T2098 mutation and one or more mutations at L2031, E2032, R2036, G2040, or F2108. In one aspect the mutation at T2098 is T2098L.
8) In an aspect the first switch domain comprises a mutation at T2098L and at E2032. In an aspect the mutation at E2032 is E2032I. In another aspect the mutation at E2032 is E2032L.
9) In an aspect the second switch domain comprises one or more mutations that enhance the formation of a complex between the first switch domain, the second switch domain, and the dimerization molecule, rapamycin, or a rapalog, e.g., RAD001. In an aspect the second switch domain comprises one or more mutations at Q53, I56, W59, Y82, G89, I90, I91, K44, P45, or H87. In an aspect, the second switch domain comprises one or more mutations at Q53, I56, W59, Y82, H87, G89, or I90.
10) In an aspect the first switch domain comprises one or more mutations that enhance the formation of a complex between the first switch domain, the second switch domain, and the dimerization molecule (e.g., rapamycin, or a rapalog, e.g., RAD001); and the second switch domain comprises one or more mutations that enhance the formation of a complex between the first switch domain, the second switch domain, and the dimerization molecule (e.g., rapamycin, or a rapalog, e.g., RAD001).

In some aspects the dimerization switch is an isolated dimerization switch, e.g., as described herein. In an aspect the invention is a preparation of a dimerization switch, e.g., as described herein. In an aspect the invention is a pharmaceutically acceptable preparation of a dimerization switch, e.g., as described herein.

In some aspects the dimerization switch comprises any one of the first or second switch domains described above, e.g., the switch domains described in 1) to 10). In some aspects the dimerization switch comprises a combination more than one of the first or second switch domains described above, e.g., the switch domains described in 1) to 10).

In some aspects of the dimerization switch, e.g., as described above, the polypeptide of (a) and the polypeptide of (b) are on separate molecules, and activation of the switch results in an intermolecular association. In some aspects of the dimerization switch, e.g., as described above, the polypeptide of (a) and the polypeptide of (b) are on the same molecule and activation of the switch results in an intramolecular association.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising one or more mutations each of which enhances formation of a complex between a first switch domain, a second switch domain (e.g., a FKBP derived switch domain), and a dimerization molecule (e.g., a rapamycin, or a rapalog, e.g., RAD001), e.g., wherein the enhancement is additive or more than additive.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a mutation at E2032, e.g., E2032I or E2032L, and at T2098, e.g., T2098L.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising the mutation E2032I, and further comprises a mutation at one or a plurality of L2031, S2035, R2036, F2039, G2040, T2098, W2101, D2102, Y2105, or F2108.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a mutation at E2032I and at T2098. In one aspect the mutation at T2098 is T2098L.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a mutation at E2032L, and further comprising a mutation at one or more of L2031, S2035, R2036, F2039, G2040, T2098, W2101, D2102, Y2105, or F2108.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a mutation at E2032L and at T2098. In one aspect the mutation at T2098 is T2098L.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a T2098 mutation and one or more mutations at L2031, E2032, R2036, G2040, or F2108. In one aspect the mutation at T2098 is T2098L.

In an aspect, the dimerization switch comprises a second switch domain comprising one or more mutations that enhance the formation of a complex between the first switch domain, the second switch domain, and the dimerization molecule, rapamycin, or a rapalog, e.g., RAD001, e.g. one or more mutations at Q53, I56, W59, Y82, G89, I90, I91, K44, P45, or H87, e.g., one or more mutations at Q53, I56, W59, Y82, H87, G89, or I90; and a first switch domain comprising a mutation at T2098L and at E2032. In an aspect the mutation at E2032 is E2032I. In another aspect the mutation at E2032 is E2032L.

In an aspect the dimerization switch comprises a first switch domain comprising T2098L and E2032I. In an aspect the dimerization switch comprises a first switch domain comprising T2098L and E2032L. In some aspects the dimerization switch further comprises a second switch domain comprising one or more mutations at Y26, F36, D37, R42, K44, P45, F46, Q53, E54, V55, I56, W59, Y82, H87, G89, I90, I91, and F99, e.g., one or more mutations at Y26, F36, D37, R42, F46, Q53, E54, V55, I56, W59, Y82, H87, G89, I90, or F99.

In some aspects the dimerization switch comprises a first switch domain that differs at no more than 1, 2, 3, 4, 5, 6, 7, 8, 9, or 10 amino acid residues from the sequence of SEQ ID NO:2.

In some aspects, the dimerization switch comprises a first switch domain comprising 30, 35, 40, 45, 50, 55, 60, 70, 75, 80, 85 or 90 amino acids of the sequence of FRB, SEQ ID NO:2.

In some aspects, the dimerization switch comprises a second switch domain that differs at no more than 1, 2, 3, 4, 5, 6, 7, 8, 9, or 10 amino acid residues from the sequence of SEQ ID NO:1 or 3.

In some aspects, the dimerization switch comprises a second switch domain comprising 30, 35, 40, 45, 50, 55, 60, 70, 75, 80, 85 or 90 amino acids of the sequence of FKBP, SEQ ID NO:1 or 3.

Aspects of the dimerization switches described herein may feature multiple switch domains, sometimes referred to herein as a multi switch. A multi switch comprises plurality of, e.g., 2, 3, 4, 5, 6, 7, 8, 9, or 10, switch domains, independently, on a first polypeptide, e.g., the polypeptide of (a), and on a second polypeptide, e.g., the polypeptide of (b), as described in the section herein entitled MULTIPLE SWITCH DOMAINS.

In some aspects, the dimerization switch comprises a polypeptide of (a) further comprising an additional switch domain, e.g., any switch domain described herein.

In some aspects, the dimerization switch comprises a polypeptide of (b) further comprising an additional switch domain, e.g., any switch domain described herein.

In some aspects, the dimerization switch comprises a polypeptide of (a) further comprising an additional switch domain; and a polypeptide of (b) further comprising an additional switch domain.

In some aspects, the dimerization switch comprises a polypeptide of (a) further comprising an additional first switch domain, e.g., any first switch domain described herein.

In some aspects, the dimerization switch comprises a polypeptide of (b) further comprising an additional second switch domain, e.g., any second switch domain described herein.

In some aspects, the dimerization switch comprises a polypeptide of (a) further comprising an additional first switch domain; and a polypeptide of (b) further comprising an additional second switch domain.

In some aspects, the dimerization switch comprises a polypeptide of (a) further comprising a second switch domain, e.g., any second switch domain described herein.

In some aspects, the dimerization switch comprises a polypeptide of (b) further comprising a first switch domain, e.g., any first switch domain described herein.

In some aspects, the dimerization switch comprises a polypeptide of (a) further comprising a second switch domain; and a polypeptide of (b) further comprising a first switch domain.

The present invention also features a dimerization switch wherein the first and second switch domains of the dimerization switch are fused to a first and second moiety. As discussed in more detail below, in aspects, the dimerization switch, in the presence of a dimerization molecule, can bring together the first and second moieties. In some aspects, the dimerization switch comprises a polypeptide comprising a first switch domain coupled, e.g., fused, to a first moiety. In some aspects, the dimerization switch comprises a polypeptide comprising a second switch domain coupled, e.g., fused, to a second moiety. In some aspects, the polypeptide comprising a first switch domain is coupled, e.g., fused, to a first moiety and the polypeptide comprising the second switch domain is coupled, e.g., fused, to a second moiety. In some aspects the first and second moieties are the same. In some aspects the first and second moieties are different.

In some aspects, the polypeptides comprising the first or second switch domains are, independently, coupled, e.g., fused, to a moiety from a pair of entities from Table 5.

In some aspects, one of the polypeptides comprising the first or second switch domain is coupled, e.g., fused, to a moiety that anchors the switch domain to a membrane.

In some aspects, one of the polypeptides comprising the first or second switch domains is coupled to, e.g., fused to, a transactivation domain of a transcription factor, e.g., the C-terminus of NFkappaB p65, and the other is coupled to, e.g., fused to, a DNA binding domain of a transcription factor, e.g., a ZFHD1 DNA binding domain.

In some aspects, one of the polypeptides comprising the first or second switch domains is coupled to, e.g., fused to, an intracellular signalling region, e.g., of Fgfr4, and the other is coupled to, e.g., fused to, another, or the same, intracellular signalling region, e.g., of Fgfr4.

In some aspects, one of the polypeptides comprising the first or second switch domains is coupled to, e.g., fused to, a functional region of a ligand, e.g., FGF2IIIb, and the other is coupled to, e.g., fused to, a functional region of a counter ligand, or receptor, e.g., FGFRIIIb.

In some aspects, one of the polypeptides comprising the first or second switch domains is coupled to, e.g., fused to, a membrane tethering domain, e.g., myristoyl group or a transmembrane domain, and the other is coupled to, e.g., fused to, another moiety, e.g., a polypeptide, e.g., an intracellular, membrane associated, or secreted polypeptide.

In some aspects, one of the polypeptides comprising the first or second switch domains is coupled to, e.g., fused to, a membrane tethering domain, e.g., myristoyl group or a transmembrane domain, and the other is coupled to, e.g., fused to, a functional region of Akt.

In some aspects, one of the polypeptides comprising the first or second switch domains is coupled to, e.g., fused to, a membrane tethering domain, e.g., myristoyl group or a transmembrane domain, and the other is coupled to, e.g., fused to, an Fgfr1 intracellular signalling domain, e.g., intracellular kinase domain,

In some aspects, one of the polypeptides comprising the first or second switch domains is coupled to, e.g., fused to, a first portion of a reporter, and the other is coupled to, e.g., fused to, an activator of the reporter.

In some aspects, one of the polypeptides comprising the first or second switch domains is coupled to, e.g., fused to, a first portion of a reporter, e.g., luciferase protein, and the other is coupled to, e.g., fused to, a second portion of a reporter, e.g., a luciferase protein.

In some aspects, one of the polypeptides comprising the first or second switch domains is coupled to, e.g., fused to, a first moiety, e.g., a polypeptide, e.g., a region of GSK3b, wherein the other switch domain, by itself or coupled, e.g., fused a second moiety, is capable of modulating, e.g., decreasing, the interaction between the first or second moiety and a third moiety, e.g., an enzyme, which can modify, e.g., degrade, activate, or phosphorylate, the first or second moiety.

In some aspects, one of the polypeptides comprising the first or second switch domains is coupled to, e.g., fused to, a moiety, e.g., a protease, kinase, or other enzyme, which can modify, e.g., covalently modify, a second moiety, and the other is coupled to, e.g., fused to, the second moiety, e.g., a polypeptide, e.g., an intracellular, membrane associated, or secreted polypeptide.

In some aspects, one of the polypeptides comprising the first or second switch domains is coupled to, e.g., fused to, a regulator of post translational modification, an active region of Sumoyltransferase U9, and the other is coupled to, e.g., fused to, a substrate of the modulator, e.g., a substrate comprising a U9 substrates, e.g., STAT1, P53, CRSP9, FOS, CSNK2B.

In some aspects, one of the polypeptides comprising the first or second switch domains is coupled to, e.g., fused to, a nuclear localization sequence (NLS).

In some aspects, one of the polypeptides comprising the first or second switch domains is coupled to, e.g., fused to, a nuclear export sequence (NES).

In some aspects, as discussed herein, one of the polypeptides comprising the first or second switch domains is coupled to, e.g., fused to, a component of a gene editing system.

In an aspect, wherein the dimerization switch is an FKBP-FRB based switch, e.g., as described herein, the dimerization molecule is an mTOR inhibitor, e.g., an allosteric mTOR inhibitor, e.g., rapamycin or a rapalog, e.g., RAD001.

In an aspect, any of the dosing regimes or formulations of an allosteric mTOR inhibitor, e.g., RAD001, described herein, can be administered to dimerize an FKBP-FRB based dimerization switch.

In an aspect, the switch is an FKBP-FRB based switch and the dimerization molecule is RAD001.

In an aspect, 0.1 to 20, 0.5 to 10, 2.5 to 7.5, 3 to 6, or about 5, mgs of RAD001 per week, e.g., delivered once per week, is administered.

In an aspect, 0.3 to 60, 1.5 to 30, 7.5 to 22.5, 9 to 18, or about 15 mgs of RAD001 in a sustained release formulation, per week, e.g., delivered once per week, is administered.

In an aspect, 0.005 to 1.5, 0.01 to 1.5, 0.1 to 1.5, 0.2 to 1.5, 0.3 to 1.5, 0.4 to 1.5, 0.5 to 1.5, 0.6 to 1.5, 0.7 to 1.5, 0.8 to 1.5, 1.0 to 1.5, 0.3 to 0.6, or about 0.5 mgs of RAD001 per day, e.g., delivered once per day, is administered.

In an aspect, 0.015 to 4.5, 0.03 to 4.5, 0.3 to 4.5, 0.6 to 4.5, 0.9 to 4.5, 1.2 to 4.5, 1.5 to 4.5, 1.8 to 4.5, 2.1 to 4.5, 2.4 to 4.5, 3.0 to 4.5, 0.9 to 1.8, or about 1.5 mgs of RAD001 in 5 a sustained release formulation, per day, e.g., delivered once once per day, is administered.

In an aspect, 0.1 to 30, 0.2 to 30, 2 to 30, 4 to 30, 6 to 30, 8 to 30, 10 to 30, 1.2 to 30, 14 to 30, 16 to 30, 20 to 30, 6 to 12, or about 10 mgs of RAD001 in a sustained release formulation, per week, e.g., delivered once once per week, is administered.

The present invention also features an isolated polypeptide, or a preparation, e.g., a pharmaceutically acceptable preparation of a peptide, comprising an FRB fragment or analog thereof, e.g., of SEQ ID NO:2, having the ability to form a complex between the FRB fragment or analog thereof, a FKBP fragment or analog thereof and a dimerization molecule, wherein the polypeptide comprises one or more of the properties described in 1) to 8), above.

In an aspect, the FRB fragment or analog thereof further comprises T2098L and E2032I.

In an aspect, the FRB fragment or analog thereof further comprises T2098L and E2032L.

In some aspects, the polypeptide comprising an FRB fragment or analog thereof is coupled, e.g., fused, to a first moiety.

In some aspects the polypeptide is coupled, e.g. fused, to a member of a pair from Table 5.

In some aspects, the polypeptide is coupled, e.g., fused, to a moiety that anchors the polypeptide to a membrane.

In some aspects, the polypeptide comprises a FRB fragment or analog thereof that differs at no more than 1, 2, 3, 4, 5, 6, 7, 8, 9, or 10 amino acid residues from the sequence of FRB, e.g., SEQ ID NO:2.

In some aspects, the polypeptide comprises a FRB fragment or analog thereof that comprises 30, 35, 40, 45, 50, 55, 60, 70, 75, 80, 85 or 90 amino acids of the sequence of FRB, e.g., SEQ ID NO:2.

Aspects of the polypeptide described herein may feature additional switch domains. In some aspects, the polypeptide comprises a plurality of, e.g., 2, 3, 4, 5, 6, 7, 8, 9, or 10, switch domains, as described in the section herein entitled MULTIPLE SWITCH DOMAINS. In some aspects the additional switch domain comprises an additional FRB fragment or analog thereof, e.g., any FRB fragment or analog thereof described herein. In some aspects the additional switch domain comprises a FKBP fragment or analog thereof, e.g., any FKBP fragment or analog thereof described herein.

The present invention also features a polypeptide, e.g., an isolated polypeptide, or a preparation, e.g., a pharmaceutically acceptable preparation of a peptide, comprising an FKBP fragment or analog thereof, e.g., of SEQ ID NO:1 or 3, wherein the polypeptide comprises a mutation that enhances the formation of a complex between the FKBP fragment or analog thereof, a FRB fragment or analog thereof, and a dimerization molecule, rapamycin, or a rapalog, e.g., RAD001; e.g., one or more mutations at Q53, I56, W59, Y82, 190, 191, K44, P45, H87 or G89, e.g., one or more mutations at Q53, I56, W59, Y82, H87, G89 or I90.

In some aspects, the polypeptide is coupled, e.g., fused, to a second moiety.

In some aspects, the polypeptide is coupled, e.g. fused, to a member of a pair from Table 5.

In some aspects, the polypeptide is coupled, e.g., fused, to a moiety that anchors the polypeptide to a membrane.

In some aspects, the polypeptide is coupled, e.g., fused, to a polypeptide, e.g., a polypeptide comprising a sequence from a intracellular, membrane bound, or secreted protein.

In some aspects, the FKBP fragment or analog thereof differs at no more than 1, 2, 3, 4, 5, 6, 7, 8, 9, or 10 amino acid residues from the sequence of SEQ ID NO:1 or 3.

In some aspects, the FKBP fragment or analog thereof comprises 30, 35, 40, 45, 50, 55, 60, 70, 75, 80, 85 or 90 amino acids of the sequence of FKBP, SEQ ID NO:1 or 3.

Aspects of the polypeptide described herein may feature additional switch domains. In some aspects, the polypeptide comprises a plurality of, e.g., 2, 3, 4, 5, 6, 7, 8, 9, or 10, switch domains, as described in the section herein entitled MULTIPLE SWITCH DOMAINS. In some aspects the additional switch domain comprises an additional FKBP fragment or analog thereof, e.g., any FRB fragment or analog thereof described herein. In some aspects the additional switch domain comprises a FRB fragment or analog thereof, e.g., any FRB fragment or analog thereof described herein.

The present invention also features a nucleic acid, e.g., an isolated nucleic acid, encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

In an aspect, the nucleic acid comprises sequence that encodes:
(a) a first switch domain described herein, or a polypeptide comprising an FRB fragment or analog thereof described herein;
(b) a second switch domain described herein, or a polypeptide comprising an FKBP fragment or analog thereof described herein; or
(a) and (b).

In an aspect, sequence encoding (a) and (b) is disposed on a single nucleic acid molecule, e.g., a viral vector, e.g., a lentivirus vector.

In an aspect, sequence encoding (a) is disposed on a first nucleic acid molecule, e.g., a viral vector, e.g., a lentivirus vector, and sequence encoding (b) is disposed on a second nucleic acid molecule, e.g., a viral vector, e.g., a lentivirus vector.

In an aspect sequence encoding (a) and sequence encoding (b) are present on a single nucleic acid molecule, are transcribed as a single transcription product, and sequence encoding a cleavable peptide, e.g., a P2A or F2A sequence, or sequence encoding an IRES, e.g., an EMCV IRES, is disposed between sequence encoding (a) and sequence encoding (b).

The present invention also features a vector system, e.g., one or more vectors, comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

In an aspect, the vector system comprises a DNA, a RNA, a plasmid, a lentivirus vector, adenoviral vector, or a retrovirus vector.

The present invention also features a cell comprising a dimerization switch described herein, a first switch domain described herein, a second switch domain described herein, a polypeptide comprising an FKBP fragment or analog thereof described herein, and/or a polypeptide comprising an FRB fragment or analog thereof described herein.

In an aspect, the cell is a human cell, e.g., a human stem cell or progenitor cell.

In an aspect, the cell is a T cell.

In an aspect, the cell is a NK cell.

The present invention also features a method of making a cell comprising a dimerization switch described herein, a first switch domain described herein, a second switch domain described herein, a polypeptide comprising an FKBP fragment or analog thereof described herein, and/or a polypeptide comprising an FRB fragment or analog thereof described herein. The method comprises introducing into the cell:

a dimerization switch described herein;

a first switch domain described herein or a polypeptide comprising an FRB fragment or analog thereof described herein;

a second switch domain described herein a polypeptide comprising an FKBP fragment or analog thereof described herein;

a nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; or

a vector system comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

The present invention also features a method of activating a dimerization switch described herein. In one aspect the method comprises contacting a composition comprising the dimerization switch with a suitable dimerization molecule. Where the dimerization switch is a FKBP/FRB based dimerization switch, the dimerization molecule may be rapamycin or a rapalog, e.g., RAD001.

In one aspect the method of activating a dimerization switch comprises providing a cell, e.g., as described herein (or a lysate or other cell free or disrupted cell preparation of the cells); and contacting the cell (or a lysate or other cell free or disrupted cell preparation of the cells) with a dimerization molecule, e.g., rapamycin or a rapalog, e.g., RAD001.

In an aspect, the dimerization molecule comprises RAD001.

In an aspect, any of the dosing regimes or formulations of an allosteric mTOR inhibitor, e.g., RAD001, described herein, can be administered to dimerize an FKBP-FRB based dimerization switch.

In an aspect, 0.1 to 20, 0.5 to 10, 2.5 to 7.5, 3 to 6, or about 5, mgs of RAD001 per week, e.g., delivered once per week, is administered.

In an aspect, 0.3 to 60, 1.5 to 30, 7.5 to 22.5, 9 to 18, or about 15 mgs of RAD001 in a sustained release formulation, per week, e.g., delivered once per week, is administered.

In an aspect, 0.005 to 1.5, 0.01 to 1.5, 0.1 to 1.5, 0.2 to 1.5, 0.3 to 1.5, 0.4 to 1.5, 0.5 to 1.5, 0.6 to 1.5, 0.7 to 1.5, 0.8 to 1.5, 1.0 to 1.5, 0.3 to 0.6, or about 0.5 mgs of RAD001 per day, e.g., delivered once per day, is administered.

In an aspect, 0.015 to 4.5, 0.03 to 4.5, 0.3 to 4.5, 0.6 to 4.5, 0.9 to 4.5, 1.2 to 4.5, 1.5 to 4.5, 1.8 to 4.5, 2.1 to 4.5, 2.4 to 4.5, 3.0 to 4.5, 0.9 to 1.8, or about 1.5 mgs of RAD001 in 5 a sustained release formulation, per day, e.g., delivered once once per day, is administered.

In an aspect, 0.1 to 30, 0.2 to 30, 2 to 30, 4 to 30, 6 to 30, 8 to 30, 10 to 30, 1.2 to 30, 14 to 30, 16 to 30, 20 to 30, 6 to 12, or about 10 mgs of RAD001 in a sustained release formulation, per week, e.g., delivered once once per week, is administered.

In an aspect, the method comprises administering a low, immune enhancing, dose of an allosteric mTOR inhibitor, e.g., RAD001.

The present invention also features a method of treating a subject, e.g., a mammal, having a disease or disorder described herein comprising administering to the subject an effective amount of a cell described herein or providing a subject comprising the cell.

In an aspect, the cell is an autologous immune cell, e.g., a T cell, a NK cell.

In an aspect, the cell is an allogeneic immune cell, e.g., a T cell, a NK cell.

In an aspect, the cell is a stem or progenitor cell.

In an aspect, the subject is a human.

In an aspect, the polypeptides comprising the first and second switch domains of the dimerization switch are coupled, e.g., fused to a transactivation domain of a transcription factor, e.g., C-terminus of NFκB p65, and to a DNA binding domain of a transcription factor, e.g., a ZFHD1 DNA binding domain.

In an aspect, the method comprises treating the subject for a disease or disorder as described herein.

In an aspect, the method comprises administering a dimerization molecule to the subject.

In an aspect, the method comprises administering a dimerization molecule comprising an mTOR inhibitor, e.g., an allosteric mTOR inhibitor, e.g., rapamycin or a rapalog, e.g., RAD001.

In an aspect, the method comprises administering a low, immune enhancing, dose of an allosteric mTOR inhibitor, e.g., RAD001.

The present invention also features a method of providing a cell, e.g., a cell described herein comprising providing an acceptor cell, e.g., a T cell from a human, to a recipient entity, e.g., a laboratory or hospital; and receiving from said entity, a cell derived from the acceptor cell, or a daughter cell thereof, wherein the cell comprises

a dimerization switch described herein;

a first switch domain described herein or a polypeptide comprising an FRB fragment or analog thereof described herein;

a second switch domain described herein a polypeptide comprising an FKBP fragment or analog thereof described herein;

a nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; or

a vector system comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

In an aspect, the receiving entity inserted into the acceptor cell,

a dimerization switch described herein;

a first switch domain described herein or a polypeptide comprising an FRB fragment or analog thereof described herein;

a second switch domain described herein a polypeptide comprising an FKBP fragment or analog thereof described herein;

a nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; or

a vector system comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

In an aspect, the method further comprises administering the cell to said human.

The present invention also features a method of providing a cell described herein comprising: receiving from an entity, e.g., a health care provider, an acceptor cell, e.g., a T cell, from a human; inserting into the acceptor cell,

a dimerization switch described herein;

a first switch domain described herein or a polypeptide comprising an FRB fragment or analog thereof described herein;

a second switch domain described herein a polypeptide comprising an FKBP fragment or analog thereof described herein;

a nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; or

a vector system comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; and

optionally, providing the cell to the entity.

The present invention also features a reaction mixture comprising any of:

a dimerization switch described herein;

a first switch domain described herein or a polypeptide comprising an FRB fragment or analog thereof described herein;

a second switch domain described herein a polypeptide comprising an FKBP fragment or analog thereof described herein;

a nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein; or

a vector system comprising nucleic acid encoding a dimerization switch described herein, a first switch domain described herein, a second switch domain described herein, a polypeptide comprising an FKBP fragment or analog thereof described herein, and/or a polypeptide comprising an FRB fragment or analog thereof described herein.

The present invention also features a gene editing dimerization switch comprising:

(a) a polypeptide comprising a first gene editing switch domain coupled to, e.g. fused to, a first moiety; and

(b) a polypeptide comprising second gene editing switch domain coupled to, e.g., fused to, a second moiety;

Wherein the first or second moiety comprises a nuclear localization sequence (NLS), and wherein the other moiety comprises a gene editing protein.

In an aspect, the gene editing dimerization switch comprises a noncovalent gene editing dimerization switch.

In an aspect the gene editing dimerization switch comprises a FKBP/FRB-based gene editing dimerization switch. In such aspects, the gene editing dimerization molecule may comprise rapamycin or a rapalog, e.g., RAD001.

In an aspect, the gene editing dimerization switch comprises a GyrB/GyrB-based gene editing dimerization switch. In such aspects, the gene editing dimerization molecule may comprise coumermycin.

In an aspect, the gene editing dimerization switch comprises a GAI/GID-1-based gene editing dimerization switch. In such aspects, the gene editing gene editing dimerization molecule may comprise gibberellin, or a giberellin analog, e.g., GA3-AM or GA3.

In an aspect the gene editing dimerization comprises a covalent gene editing dimerization switch.

In an aspect, the covalent gene editing dimerization switch is a Halo-tag/SNAP-tag-based gene editing dimerization switch. In such aspects, the gene editing dimerization molecule may comprise HaXS.

In an aspect, the first gene editing switch domain comprises an FRB fragment or analog thereof and the second gene editing switch domain comprises an FKBP fragment or analog thereof. In such aspects, a gene editing dimerization molecule may comprise rapamycin or a rapalog, e.g., RAD001

In an aspect, the gene editing dimerization switch comprises one or more of the gene editing switch domains 1) to 10), below:
1) In an aspect, the first gene editing switch domain comprises one or more mutations each of which enhances formation of a complex between a first gene editing switch domain, a second gene editing switch domain (e.g., a FKBP derived switch domain), and a gene editing dimerization molecule (e.g., a rapamycin, or a rapalog, e.g., RAD001). In an aspect, the enhancement is additive or more than additive.
2) In an aspect, the first gene editing switch domain comprises a mutation at E2032, e.g., E2032I or E2032L, and at T2098, e.g., T2098L.
3) In an aspect, the gene editing first switch domain comprises the mutation E2032I, and further comprises a mutation at one or a plurality of L2031, S2035, R2036, F2039, G2040, T2098, W2101, D2102, Y2105, or F2108.
4) In an aspect, the first gene editing switch domain comprises a mutation at E2032I and at T2098. In one aspect the mutation at T2098 is T2098L.
5) In an aspect, the first gene editing switch domain comprises the mutation at E2032L, and further comprises a mutation at one or more of L2031, S2035, R2036, F2039, G2040, T2098, W2101, D2102, Y2105, or F2108.
6) In an aspect, the first gene editing switch domain comprises a mutation at E2032L and at T2098. In one aspect the mutation at T2098 is T2098L.
7) In an aspect, the first gene editing switch domain comprises a T2098 mutation and one or more mutations at L2031, E2032, R2036, G2040, or F2108. In one aspect the mutation at T2098 is T2098L.
8) In an aspect the gene editing first switch domain comprises a mutation at T2098L and at E2032. In an aspect the mutation at E2032 is E2032I. In another aspect the mutation at E2032 is E2032L.
9) In an aspect the second gene editing switch domain comprises one or more mutations that enhance the formation of a complex between the first gene editing switch domain, the second gene editing switch domain, and the gene editing dimerization molecule, rapamycin, or a rapalog, e.g., RAD001. In an aspect the second gene editing switch domain comprises one or more mutations at Q53, I56, W59, Y82, G89, I90, I91, K44, P45, or H87. In an aspect, the second gene editing switch domain comprises one or more mutations at Q53, I56, W59, Y82, H87, G89, or I90.
10) In an aspect the first gene editing switch domain comprises one or more mutations that enhance the formation of a complex between the first gene editing switch domain, the second gene editing switch domain, and the gene editing dimerization molecule, rapamycin, or a rapalog, e.g., RAD001; and (B) the second gene editing switch domain comprises one or more mutations that enhance the formation of a complex between the first gene editing switch domain, the second gene editing switch domain, and the gene editing dimerization molecule, rapamycin, or a rapalog, e.g., RAD001.

In an aspect, the gene editing dimerization switch comprises 9) and 1).

In an aspect, the gene editing dimerization switch comprises 9) and 2).

In an aspect, the gene editing dimerization switch comprises 9) and 3).

In an aspect, the gene editing dimerization switch comprises 9) and 4).

In an aspect, the gene editing dimerization switch comprises 9) and 5).

In an aspect, the gene editing dimerization switch comprises 9) and 6).

In an aspect, the gene editing dimerization switch comprises 9) and 7).

In an aspect, the gene editing dimerization switch comprises 9) and 8).

In an aspect, the gene editing dimerization switch comprises a first gene editing switch domain that comprises a first switch domain as described herein or a polypeptide comprising an FRB fragment or analog thereof as described herein.

In an aspect, the gene editing dimerization switch comprises a second gene editing switch domain that comprises a second switch domain as described herein or a polypeptide comprising an FKBP fragment or analog thereof as described herein.

In some aspects the gene editing dimerization switch comprises a first gene editing switch domain that differs at no more than 1, 2, 3, 4, 5, 6, 7, 8, 9, or 10 amino acid residues from the sequence of SEQ ID NO:2.

In some aspects, the gene editing dimerization switch comprises a first gene editing switch domain comprising 30, 35, 40, 45, 50, 55, 60, 70, 75, 80, 85 or 90 amino acids of the sequence of FRB, SEQ ID NO:2.

In some aspects, the gene editing dimerization switch comprises a second gene editing switch domain that differs at no more than 1, 2, 3, 4, 5, 6, 7, 8, 9, or 10 amino acid residues from the sequence of SEQ ID NO:1 or 3.

In some aspects, the gene editing dimerization switch comprises a second gene editing switch domain comprising 30, 35, 40, 45, 50, 55, 60, 70, 75, 80, 85 or 90 amino acids of the sequence of FKBP, SEQ ID NO:1 or 3.

In an aspect, the gene editing dimerization switch comprises a first gene editing switch domain that comprises a first switch domain as described herein or a polypeptide comprising an FRB fragment or analog thereof as described herein and a. second gene editing switch domain that comprises a second switch domain as described herein or a polypeptide comprising an FKBP fragment or analog thereof as described herein.

In an aspect, the gene editing protein comprises a zinc finger nuclease.

In an aspect, the gene editing protein comprises a transcription activator-like effector nuclease (TALEN).

In an aspect, the gene editing protein comprises a CRISPR-associated nuclease, e.g., Cas9 or dCas9.

In an aspect, the gene editing protein comprises a meganuclease.

The present invention also features a gene editing dimerization switch comprising:

(a) a polypeptide comprising a first gene editing switch domain coupled to, e.g. fused to, a first moiety; and

(b) a polypeptide comprising second gene editing switch domain coupled to, e.g., fused to, a second moiety;

Wherein the first or second moiety comprises a DNA-binding domain and the other moiety comprises a DNA-modifying domain.

In one aspect, the DNA-binding domain is a zinc finger or engineered zinc finger.

In one aspect, the DNA-binding domain is a transcription activator-like effector (TALE).

In one aspect, the DNA-binding domain is a DNA-binding domain of a Cas9, e.g., dCas9.

In one aspect, the DNA-modifying domain is a polypeptide having nuclease activity.

In one aspect, the DNA-modifying domain is a nuclease half-domain.

In one aspect, the nuclease half-domain is FokI or a derivative thereof.

In an aspect, the first gene editing switch domain comprises a sequence derived from FRB having the ability to form a complex with an FKBP and AP21967, e.g., a sequence comprising a lysine at residue 2098.

In an aspect the first gene editing switch domain comprises a sequence derived from FRB having the ability to form a complex with an FKBP and AP21967, e.g., a sequence comprising a lysine at residue 2098; and, the second gene editing switch domain comprises a sequence derived from FKBP having the ability to form a complex with an FRB and AP21967.

In an aspect, the gene editing dimerization molecule is a rapamycin analogue, e.g., AP21967.

In an aspect, the gene editing dimerization switch comprises a GyrB-GyrB-based gene editing dimerization switch, e.g., as described herein.

In an aspect, the first or second gene editing switch domain comprises a coumermycin binding sequence having at least 80, 85, 90, 95, 98, or 99% identity with the 24 K Da amino terminal sub-domain of GyrB.

In an aspect, the first or second gene editing switch domain comprises a coumermycin binding sequence that differs by no more than 10, 9, 8, 7, 6, 5, 4, 3, 2, or 1 amino acid residues from the corresponding sequence of 24 K Da amino terminal sub-domain of GyrB.

In an aspect, the first or second gene editing switch domain comprises a coumermycin binding sequence from the 24 K Da amino terminal sub-domain of GyrB.

In an aspect, the first or second gene editing switch domain comprises the 24 K Da amino terminal sub-domain of GyrB.

In an aspect, the gene editing dimerization molecule is a coumermycin.

In an aspect, the gene editing dimerization switch comprises a GAI-GID1-based gene editing dimerization switch, e.g., as described herein.

In an aspect, the first or second gene editing switch domain comprises a gibberellin, or gibberellin analog, e.g., GA3, binding sequence having at least 80, 85, 90, 95, 98, or 99% identity with GID1, and the other gene editing switch domain comprises a GAI having at least 80, 85, 90, 95, 98, or 99% identity with GAI.

In an aspect, the first or second gene editing switch domain comprises a gibberellin, or gibberellin analog, e.g., GA3, binding sequence that differs by no more than 10, 9, 8, 7, 6, 5, 4, 3, 2, or 1 amino acid residues from the corresponding sequence of a GID1 described herein, and the other gene editing switch domain comprises a polypeptide that differs by no more than 10, 9, 8, 7, 6, 5, 4, 3, 2, or 1 amino acid residues from the corresponding sequence of a GAI described herein.

In an aspect, the gene editing dimerization molecule is gibberellin, or a giberellin analog, e.g., GA3-AM or GA3.

In an aspect, the first and/or second gene editing switch domains comprise a polypeptide having affinity for an antibody molecule, or a non-antibody scaffold, e.g., a fribronectin or adnectin.

In an aspect, the dimerization molecule is an antibody molecule, or a non-antibody scaffold, e.g., a fribronectin or adnectin having specific affinity for one or both of the first and second gene editing switch domains.

In an aspect, the gene editing dimerization switch comprises a covalent switch.

In an aspect, the gene editing dimerization switch comprises a Halo-tag/SNAP-tag-based gene editing dimerization switch.

In an aspect, the first or second gene editing dimerization switch domain comprises a Halo-tag comprising at least 80, 85, 90, 95, 98, or 99% identity with SEQ ID NO: 38, and the other gene editing switch domain comprises a SNAP-tag having at least 80, 85, 90, 95, 98, or 99% identity with SEQ ID NO: 39.

In an aspect, the first or second gene editing dimerization switch domain comprises a Halo-tag that differs by no more than 10, 9, 8, 7, 6, 5, 4, 3, 2, or 1 amino acid residues from SEQ ID NO: 38, and the other gene editing switch domain comprises a SNAP-tag that differs by no more than 10, 9, 8, 7, 6, 5, 4, 3, 2, or 1 amino acid residues from SEQ ID: 39.

In an aspect, the gene editing dimerization molecule is HaXS

In an aspect, the gene editing dimerization switch comprises an FKBP/FRB based dimerization switch, e.g., as described herein.

In an aspect, the gene editing dimerization switch comprises one or more of the gene editing switch domains 1) to 10), below:
1) In an aspect, the first gene editing switch domain comprises one or more mutations each of which enhances formation of a complex between a first gene editing switch domain, a second gene editing switch domain (e.g., a FKBP derived switch domain), and a gene editing dimerization molecule (e.g., a rapamycin, or a rapalog, e.g., RAD001). In an aspect, the enhancement is additive or more than additive.
2) In an aspect, the first gene editing switch domain comprises a mutation at E2032, e.g., E2032I or E2032L, and at T2098, e.g., T2098L.
3) In an aspect, the gene editing first switch domain comprises the mutation E2032I, and further comprises a mutation at one or a plurality of L2031, S2035, R2036, F2039, G2040, T2098, W2101, D2102, Y2105, or F2108.
4) In an aspect, the first gene editing switch domain comprises a mutation at E2032I and at T2098. In one aspect the mutation at T2098 is T2098L.
5) In an aspect, the first gene editing switch domain comprises the mutation at E2032L, and further comprises a mutation at one or more of L2031, S2035, R2036, F2039, G2040, T2098, W2101, D2102, Y2105, or F2108.
6) In an aspect, the first gene editing switch domain comprises a mutation at E2032L and at T2098. In one aspect the mutation at T2098 is T2098L.
7) In an aspect, the first gene editing switch domain comprises a T2098 mutation and one or more mutations at L2031, E2032, R2036, G2040, or F2108. In one aspect the mutation at T2098 is T2098L.
8) In an aspect the gene editing first switch domain comprises a mutation at T2098L and at E2032. In an aspect the mutation at E2032 is E2032I. In another aspect the mutation at E2032 is E2032L.
9) In an aspect the second gene editing switch domain comprises one or more mutations that enhance the formation of a complex between the first gene editing switch domain, the second gene editing switch domain, and the gene editing dimerization molecule, rapamycin, or a rapalog, e.g., RAD001. In an aspect the second gene editing switch domain comprises one or more mutations at Q53, I56, W59, Y82, G89, I90, I91, K44, P45, or H87. In an aspect, the second gene editing switch domain comprises one or more mutations at Q53, I56, W59, Y82, H87, G89, or I90.
10) In an aspect, the first gene editing switch domain comprises one or more mutations that enhance the formation of a complex between the first gene editing switch domain, the second gene editing switch domain, and the gene editing dimerization molecule, rapamycin, or a rapalog, e.g., RAD001; and (B) the second gene editing switch domain comprises one or more mutations that enhance the formation of a complex between the first gene editing switch domain, the second gene editing switch domain, and the gene editing dimerization molecule, rapamycin, or a rapalog, e.g., RAD001.

In an aspect, the gene editing dimerization switch comprises 9) and 1).

In an aspect, the gene editing dimerization switch comprises 9) and 2).

In an aspect, the gene editing dimerization switch comprises 9) and 3).

In an aspect, the gene editing dimerization switch comprises 9) and 4).

In an aspect, the gene editing dimerization switch comprises 9) and 5).

In an aspect, the gene editing dimerization switch comprises 9) and 6).

In an aspect, the gene editing dimerization switch comprises 9) and 7).

In an aspect, the gene editing dimerization switch comprises 9) and 8).

In an aspect, the first gene editing switch domain comprises a first switch domain as described herein, or a polypeptide comprising an FRB fragment or analog thereof as described herein.

In an aspect, the second gene editing switch domain comprises a second switch domain as described herein, or a polypeptide comprising a FKBP fragment or analog thereof as described herein.

In some aspects the gene editing dimerization switch comprises a first gene editing switch domain that differs at no more than 1, 2, 3, 4, 5, 6, 7, 8, 9, or 10 amino acid residues from the sequence of SEQ ID NO:2.

In some aspects, the gene editing dimerization switch comprises a first gene editing switch domain comprising 30, 35, 40, 45, 50, 55, 60, 70, 75, 80, 85 or 90 amino acids of the sequence of FRB, SEQ ID NO:2.

In some aspects, the gene editing dimerization switch comprises a second gene editing switch domain that differs at no more than 1, 2, 3, 4, 5, 6, 7, 8, 9, or 10 amino acid residues from the sequence of SEQ ID NO:1 or 3.

In some aspects, the gene editing dimerization switch comprises a second gene editing switch domain comprising 30, 35, 40, 45, 50, 55, 60, 70, 75, 80, 85 or 90 amino acids of the sequence of FKBP, SEQ ID NO:1 or 3.

In an aspect, the first gene editing switch domain comprises a first switch domain as described herein, or a polypeptide comprising an FRB fragment or analog thereof as described herein and the second gene editing switch domain comprises a second switch domain as described herein, or a polypeptide comprising a FKBP fragment or analog thereof as described herein.

In aspects, a polypeptide comprising a gene editing switch domain may feature additional switch domains. In some aspects, the polypeptide comprises a plurality of, e.g., 2, 3, 4, 5, 6, 7, 8, 9, or 10, gene editing switch domains, as described in the section herein entitled MULTIPLE SWITCH DOMAINS. In some aspects the additional gene editing switch domain comprises an additional FKBP fragment or analog thereof, e.g., any FRB fragment or analog thereof described herein. In some aspects the additional gene editing switch domain comprises a FRB fragment or analog thereof, e.g., any FRB fragment or analog thereof described herein.

In an aspect, the polypeptide comprising the first and/or second gene editing switch domain further comprises a NLS.

In an aspect, the gene editing dimerization molecule is a rapamycin or a rapalog, e.g., RAD001.

In an aspect, the gene editing dimerization molecule is RAD001.

In an aspect, any of the dosing regimes or formulations of an allosteric mTOR inhibitor, e.g., RAD001, described herein, can be administered to dimerize an FKBP-FRB based dimerization switch.

In an aspect, 0.1 to 20, 0.5 to 10, 2.5 to 7.5, 3 to 6, or about 5, mgs of RAD001 per week, e.g., delivered once per week, is administered.

In an aspect, 0.3 to 60, 1.5 to 30, 7.5 to 22.5, 9 to 18, or about 15 mgs of RAD001 in a sustained release formulation, per week, e.g., delivered once per week, is administered.

In an aspect, 0.005 to 1.5, 0.01 to 1.5, 0.1 to 1.5, 0.2 to 1.5, 0.3 to 1.5, 0.4 to 1.5, 0.5 to 1.5, 0.6 to 1.5, 0.7 to 1.5, 0.8 to 1.5, 1.0 to 1.5, 0.3 to 0.6, or about 0.5 mgs of RAD001 per day, e.g., delivered once per day, is administered.

In an aspect, 0.015 to 4.5, 0.03 to 4.5, 0.3 to 4.5, 0.6 to 4.5, 0.9 to 4.5, 1.2 to 4.5, 1.5 to 4.5, 1.8 to 4.5, 2.1 to 4.5, 2.4 to 4.5, 3.0 to 4.5, 0.9 to 1.8, or about 1.5 mgs of RAD001 in 5 a sustained release formulation, per day, e.g., delivered once once per day, is administered. In an aspect, 0.1 to 30, 0.2 to 30, 2 to 30, 4 to 30, 6 to 30, 8 to 30, 10 to 30, 1.2 to 30, 14 to 30, 16 to 30, 20 to 30, 6 to 12, or about 10 mgs of RAD001 in a sustained release formulation, per week, e.g., delivered once once per week, is administered.

In an aspect, the gene editing dimerization switch may be dimerized using a low, immune enhancing, dose of an allosteric mTOR inhibitor, e.g., RAD001.

The present invention also features a nucleic acid, e.g., an isolated nucleic acid, comprising sequence that encodes a gene editing dimerization switch as described herein.

In an aspect the sequence encoding the polypeptide comprising the first gene editing switch domain and the sequence encoding the polypeptide comprising the second gene editing switch domain is disposed on a single nucleic acid molecule, e.g., a viral vector, e.g., a lentivirus vector.

In an aspect the sequence encoding the polypeptide comprising the first gene editing switch domain is disposed on a first nucleic acid molecule, e.g., a viral vector, e.g., a lentivirus vector, and the sequence encoding the polypeptide comprising the second gene editing switch domain is disposed on a second nucleic acid molecule, e.g., a viral vector, e.g., a lentivirus vector.

The present invention also features a vector system, e.g., one or more vectors, comprising a nucleic acid comprising sequence that encodes a gene editing dimerization switch as described herein.

In an aspect, the vector system comprises a DNA, a RNA, a plasmid, a lentivirus vector, adenoviral vector, or a retrovirus vector.

The present invention also features a method of modulating expression of an endogenous gene in a cell comprising administering to the cell a gene editing dimerization switch described herein; a nucleic acid encoding a gene editing dimerization switch described herein; or a vector system comprising a nucleic acid comprising sequence that encodes a gene editing dimerization switch as described herein; and contacting the cell with a gene editing dimerization molecule, such that expression of the endogenous gene is modulated.

In an aspect, the gene editing dimerization molecule comprises RAD001.

In an aspect, expression of a gene in a cell is repressed.

In an aspect, expression of a gene in a cell is activated.

The present invention also features a method of modifying an endogenous nucleic acid sequence, e.g., a gene, in a cell, comprising administering to the cell a gene editing dimerization switch described herein; a nucleic acid encoding a gene editing dimerization switch described herein; or a vector system comprising a nucleic acid comprising sequence that encodes a gene editing dimerization switch as described herein; and contacting the cell with a gene editing dimerization molecule, such that an endogenous nucleic acid sequence, e.g., a gene, in a cell is modified.

In an aspect, the modifying of an endogenous nucleic acid sequence comprises the deletion one or more nucleic acid residues.

In an aspect, the modifying of an endogenous nucleic acid sequence comprises the replacement of one or more endogenous nucleic acid residues with nucleic acids from a donor nucleic acid molecule.

In an aspect, the administering to the cell is performed in vivo.

In an aspect, the administering to the cell is performed in vitro.

In an aspect, the administering to the cell is performed ex vivo.

The present invention also features a cell comprising a gene editing dimerization switch as described herein, a nucleic acid encoding a gene editing dimerization switch as described herein; or a vector system comprising nucleic acid encoding a gene editing dimerization switch as described herein.

In an aspect, expression of one or more endogenous genes has been modulated by a method of modulating expression of an endogenous gene in a cell described herein.

In an aspect, one or more endogenous nucleic acid sequences, e.g., genes, have been modified by a method of modifying an endogenous nucleic acid sequence, e.g., a gene, in a cell described herein.

In an aspect, the one or more endogenous genes comprises an HLA gene.

In an aspect, the one or more endogenous genes comprises a TCR gene, e.g., TCRα or TCRβ.

In an aspect, the one or more endogenous genes comprises an inhibitory molecule selected from the group consisting of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4 and TGFR beta.

In an aspect, the cell is descended from a cell described herein, e.g. a daughter cell.

The present invention also features a method of treating a subject, e.g., a mammal having a disease associated with abberant gene expression, e.g., a disease described herein, comprising administering to the subject an effective amount of a gene editing dimerization switch described herein; a nucleic acid encoding a gene editing dimerization switch described herein, or a cell as described herein.

In an aspect the disease associated with abberant gene expression is a genetic disorder.

In an aspect the disease associated with abberant gene expression is a cancer.

The present invention also features a method of treating a subject, e.g., a mammal, having a lysosomal storage disorder, e.g., as described herein, comprising administering to the subject an effective amount of a gene editing dimerization switch described herein; a nucleic acid encoding a gene editing dimerization switch described herein, or a cell as described herein. Any of the mutations herein can be replaced with a mutation that is a conservative replacement of the designated mutation.

### BRIEF DESCRIPTION OF THE DRAWINGS

FIG. 1 is a graphic representation of the ternary complex between FKBP12, FRB and rapamycin, and was derived from RCSB Protein Data Bank code 2FAP. The dotted area represents the pocket surrounding the interface with the rapamycin or rapalog. Residues on FRB (labeled “A”) or FKBP (labeled “B”) that are in proximity of the rapamycin or rapalog or mediate interaction with rapamycin or the rapalog are circled and the amino acid position number is listed.

FIG. 2 shows the amino acid distribution of the NKK library used to generate libraries of FRB mutants. The different amino acids are listed on the x-axis, and the percent represented in the library is shown on the y-axis.

FIGS. 3A and 3B show the protein expression results from each of the different mutant FRB libraries. The 11 different mutant FRB libraries are listed on the x-axis. In FIG. 3A, the y-axis shows the percent of wells expressing the mutant FRB. In FIG. 3B, the y-axis shows the average protein concentration determined for each library.

FIGS. 4A, 4B, 4C, 4D, and 4E show the binding curves for the EC50 competition binding assay for FRB mutants: E2032L (FIG. 4A), E2032I (FIG. 4B), T2098L (FIG. 4C), E2032L, T2098L (FIG. 4D), and E2032I, T2098L (FIG. 4E).

FIGS. 5A, 5B, and 5C show the binding curves for the EC50 direct binding assay for FRB mutants: E2032L (FIG. 5A), E2032I (FIG. 5B), and T2098L (FIG. 5C).

FIGS. 6A, 6B, and 6C are schematic representations showing different configurations of regulatable receptor tyrosine kinases (RTKs) involved in cell proliferation for tissue regeneration and repair via the P13K/AKT signaling pathway. The FKBP/FRB switch domains are conjugated to RTKs extracellularly (FIG. 6A), intracellularly (FIG. 6B), and intracellularly, without a transmembrane domain or membrane anchor (FIG. 6C).

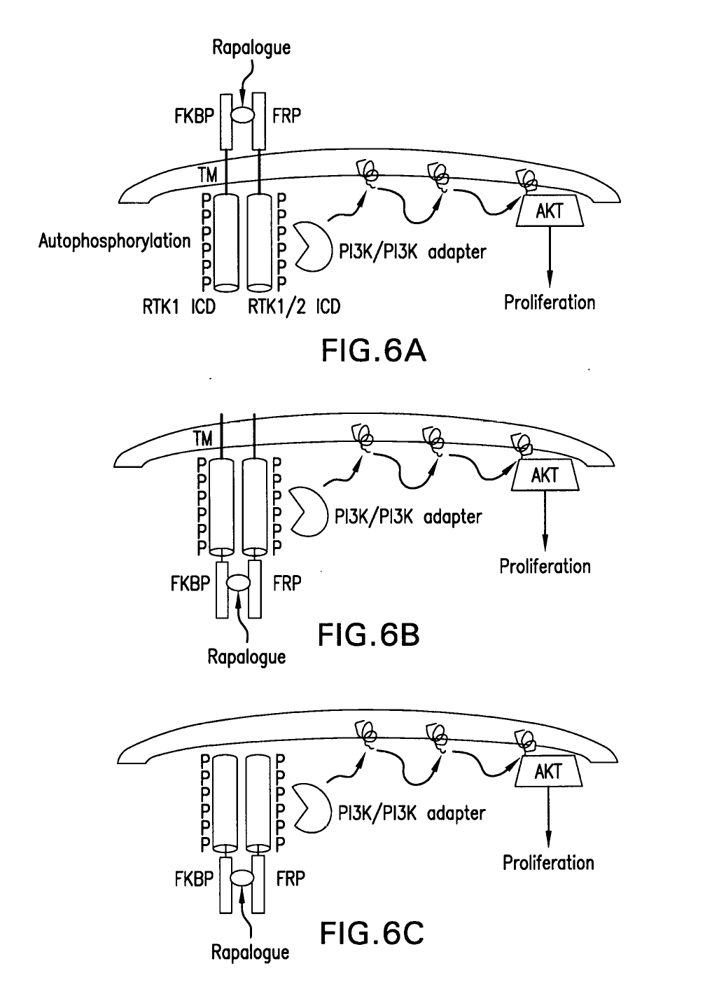

FIG. 7 is a schematic representation showing the configuration of elements on FGFRIIIb constructs that can be used to regulate P13K/AKT signaling, as described in FIGS. 6A, 6B, and 6C.

FIG. 8 is a schematic representation showing a configuration of a regulatable gene editing protein. “GESD1” stands for a first gene editing switch domain, and “GESD2” stands for a second gene editing switch domain.

FIGS. 9A, 9B and 9C are schematic representations showing different configurations of regulatable gene editing systems. The gene editing switch domains are coupled, e.g., fused, to the DNA-binding and DNA-modifying domains of the gene editing system. Exemplary DNA-modifying domains include a FokI or FokI half domain (referred to as “FokI” in FIGS. 9A and 9B) or the nuclease domain of Cas9 (FIG. 9C). Exemplary DNA-modifying domains include a zinc finger or engineered zinc finger (referred to as “Zinc Finger” in FIG. 9A), a TALE (FIG. 9B), and a domain of Cas9 responsible for DNA binding or guide RNA binding (referred to in FIG. 9C as “Cas9 DNA- or RNA-binding domain”). In these examples, the gene editing switch comprises a first or second gene editing switch domain comprising a FKBP fragment or analog thereof (“FKBP”), the other gene editing switch domain comprising a FRB fragment or analog thereof (“FRB”) and a RAD001 gene editing dimerization molecule.

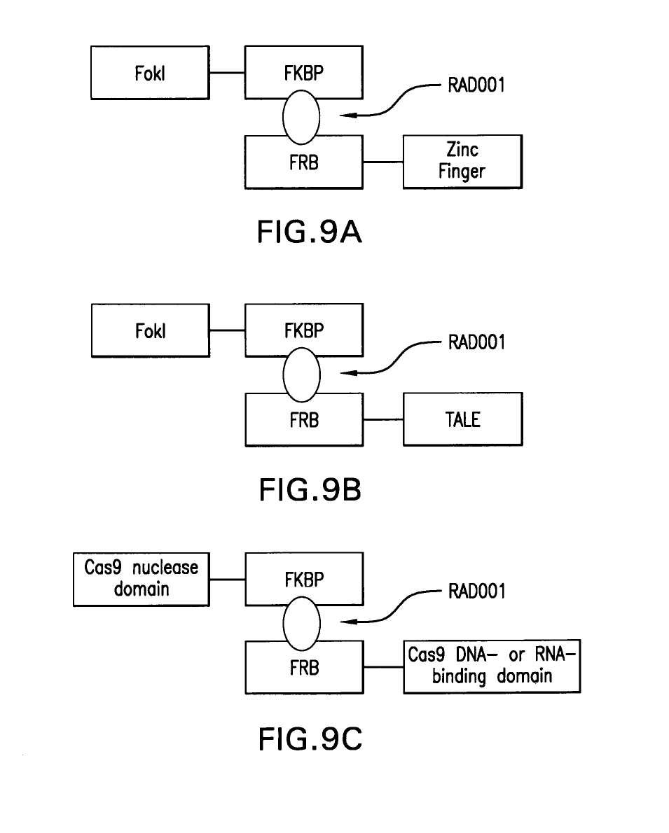

### DESCRIPTION

### Definitions

Unless defined otherwise, all technical and scientific terms used herein have the same meaning as commonly understood by one of ordinary skill in the art to which the invention pertains.

“A” and “an” as used herein, refers to one or to more than one (i.e., to at least one) of the grammatical object of the article. By way of example, “an element” means one element or more than one element.

The term “about” as used herein, when referring to a measurable value such as an amount, a temporal duration, and the like, is meant to encompass variations of ±20% or in some aspects ±10%, or in some aspects ±5%, or in some aspects ±1%, or in some aspects ±0.1% from the specified value, as such variations are appropriate to perform the disclosed methods.

The term “amino acid” as used herein, refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” as used herein, applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

The term “optimized” as used herein refers to a nucleotide sequence has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” as used herein in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “derived” as used herein, indicates a relationship between a first and a second molecule. It generally refers to structural similarity between the first molecule and a second molecule and does not conotate or include a process or source limitation on a first molecule that is derived from a second molecule. For example, in the case of an FRB fragment or analog thereof that is derived from a FRB molecule, the FRB fragment or analog thereof retains sufficient FRB structure such that is has the required function, namely, the ability bind to or associate with, in the presence of a dimerization molecule (e.g., rapamycin or a rapalog, e.g., RAD001) FKBP and/or a FKBP fragment or analog thereof. It does not conotate or include a limitation to a particular process of producing the FRB fragment or analog thereof, e.g., it does not mean that, to provide the FRB fragment or analog thereof, one must start with a FRB sequence and delete unwanted sequence, or impose mutations, to arrive at the FRB fragment or analog thereof.

The term “endogenous” refers to any material from or produced inside an organism, cell, tissue or system.

The term “exogenous” refers to any material introduced from or produced outside anorganism, cell, tissue or system.

The term “expression” refers to the transcription and/or translation of a particular nucleotide sequence driven by a promoter.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “tumor” as used herein, refers to neoplastic cell growth and proliferation, whether malignant or benign, and all pre-cancerous and cancerous cells and tissues.

The term “anti-tumor activity” as used herein, refers to a reduction in the rate of tumor cell proliferation, viability, or metastatic activity. A possible way of showing anti-tumor activity is to show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The term “malignancy” as used herein, refers to a non-benign tumor or a cancer. As used herein, the term “cancer” includes a malignancy characterized by deregulated or uncontrolled cell growth. Exemplary cancers include: carcinomas, sarcomas, leukemias, and lymphomas.

The term “cancer” as used herein, includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “pharmaceutically acceptable carrier” as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention, as used herein, refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting aspect, the term “a therapeutically effective amount” refers to the amount of the compound of the present invention that, when administered to a subject, is effective to at least partially alleviate, inhibit, prevent and/or ameliorate a condition, or a disorder or a disease, or at least partially inhibit activity of a targeted enzyme or receptor.

The terms “inhibit”, “inhibition” or “inhibiting” as used herein, refers to the reduction or suppression of a given condition, symptom, or disorder, or disease, or a significant decrease in the baseline activity of a biological activity or process.

The terms “treat”, “treating” or “treatment” of any disease or disorder, as used herein, refers in one aspect, to ameliorating the disease or disorder (i.e., slowing or arresting or reducing the development of the disease or at least one of the clinical symptoms thereof). In another aspect “treat”, “treating” or “treatment” refers to alleviating or ameliorating at least one physical parameter including those which may not be discernible by the patient. In yet another aspect, “treat”, “treating” or “treatment” refers to modulating the disease or disorder, either physically, (e.g., stabilization of a discernible symptom), physiologically, (e.g., stabilization of a physical parameter), or both. In yet another aspect, “treat”, “treating” or “treatment” refers to preventing or delaying the onset or development or progression of the disease or disorder.

As used herein, a subject is “in need of” a treatment if such subject would benefit biologically, medically or in quality of life from such treatment.

The term “coupled” or “coupled to” as used herein in the context of molecular interactions, refers to the association of two or more molecules or molecular complexes. The association can be through one or multiple covalent bonds, or non-covalent interactions and can include chelation. Various linkers, known in the art, can be employed in order to connect members of a molecular complex of the present invention. Additionally, a molecular complex of the present invention can be provided in the form of a fusion protein. The term “fusion protein” as used herein refers to proteins created through the joining of two or more genes or gene fragments which originally coded for separate proteins. Translation of the fusion gene results in a single protein with functional properties derived from each of the original proteins.

The term “affinity” as used herein, refers to the strength of interaction between two molecules or molecular complexes. Affinity can be measured, for example, by an affinity constant, a dissociation constant, or a competition binding assay by methods known in the art, e.g., Briggs, G. E., and Haldane, J. B. (1925) Biochem J 19:338-339; J Clin Invest. (1960) 39(7): 1157-1175, Yapici et al. Chembiochem (2012) 13: 553-489, or e.g., by a method described herein in the section entitled SCREENING ASSAYS.

The term “greater affinity” as used herein, indicates that a moiety, e.g., a first switch domain, binds more tightly (i.e., has stronger interaction) to its binding partner (e.g., a second switch domain) than a reference moiety, or than the same moiety in a reference condition, e.g., with a lower dissociation constant.

The terms “enhance,” “enhanced” and/or “enhances” as it relates to the formation of a dimerization complex, e.g., a complex among a first switch domain, a second switch domain, and a dimerization molecule (rapamycin or a rapalog, for example RAD001), indicates that a dimerization complex is more favorably formed and/or more stable due to greater affinity of one or more binding partners in the complex, or the complex as a whole.

The term “moiety” as used herein, refers to any molecular entity that can be coupled to (e.g., fused to) a switch domain or a gene editing switch domain. For example, a moiety can be a polypeptide, a chemical or drug molecule, or a nucleic acid, e.g., a DNA or RNA, or a combination thereof.

The term “dimerization molecule,” as used herein, refers to a molecule that promotes the association of a first switch domain with a second switch domain of an FKBP/FRB-based switch described herein. In some aspects, the dimerization molecule does not naturally occur in the subject, or does not occur in concentrations that would result in significant dimerization. In some aspects, the dimerization molecule is a small molecule, e.g., rapamycin or a rapalog, e.g., RAD001. In some aspects, the first and second switch domains of the FKBP/FRB-based switch described herein associate together in the presence of a small molecule dimerization molecule e.g., rapamycin or a rapalog.

The term “gene editing dimerization molecule” as used herein, refers to a molecule that promotes the association of a first gene editing switch domain with a second gene editing switch domain, e.g., as described herein. In some aspects the gene editing dimerization molecule is a “dimerization molecule.” In some aspects, the gene editing dimerization molecule is a small molecule, e.g., rapamycin or a rapalog. In some aspects, the gene editing dimerization molecule is a polypeptide. In some aspects, the gene editing dimerization molecule is an antibody molecule, e.g., antibody or antigen-binding fragment thereof, wherein the antibody or antigen-binding fragment thereof can be monospecific, bispecific, or multispecific. In some aspects, the first and second gene editing switch domains of a homodimerization gene editing dimerization switch or heterodimerization gene editing dimerization switch associate together in the presence of a small molecule gene editing dimerization molecule e.g., rapamycin or a rapalog. In some aspects, the first and second gene editing switch domains of a homodimerization gene editing dimerization switch or heterodimerization gene editing dimerization switch associate together in the presence of a polypeptide gene editing dimerization molecule. In some aspects, the first and second gene editing switch domains of a homodimerization gene editing dimerization switch or heterodimerization gene editing dimerization switch associate together in the presence of a multimeric peptide gene editing dimerization molecule. In some aspects, the first and second gene editing switch domains of a homodimerization gene editing dimerization switch or heterodimerization gene editing dimerization switch associate together in the presence of an antibody molecule gene editing dimerization molecule.

Generally, a gene editing dimerization molecule will promote the association of at least two gene editing switch domains (and thereby the association of moieties coupled to (e.g., fused to) the gene editing switch domains). In some aspects the gene editing dimerization molecule has a valency of greater than two, e.g., it is multi-valent, and binds, and thus clusters or binds to, more than two gene editing switch domains. For example, a gene editing dimerization molecule can comprise a plurality, e.g., at least 2, 3, 4, 5, 6, 7, 8, 9 or 10, binding domains, each of which can bind a gene editing switch domain.

The term “switch domain,” as used herein, refers to an FKBP or FRB derived polypeptide. In the presence of a dimerization molecule, e.g., RAD001, an FKPB derived switch domain associates with an FRB derived switch domain. The association results in a functional coupling of a first moiety coupled to, e.g., fused to, a first switch domain, and a second moiety coupled to, e.g., fused to, a second switch domain. A first and second switch domain are collectively referred to as a “dimerization switch.”

The term “gene editing switch domain,” as used herein, refers to a member, typically a polypeptide-based member, that, in the presence of a gene editing dimerization molecule, associates with another gene editing switch domain. The association results in a functional coupling of a first moiety coupled to (e.g., fused to) a first gene editing switch domain, and a second moiety coupled to (e.g., fused to) a second gene editing switch domain. A first and second gene editing switch domain are collectively referred to as a “gene editing dimerization switch.” In some aspects, the first and second gene editing switch domains are the same as one another, e.g., they are polypeptides having the same primary amino acid sequence, and are referred to collectively as a homodimerization switch. In some aspects, the first and second gene editing switch domains are different from one another, e.g., they are polypeptides having different primary amino acid sequence, and are referred to collectively as a heterodimerization switch. In some aspects, the gene editing switch domain is a “switch domain.” In some aspects, the gene editing switch domain is a polypeptide-based moiety, e.g., FKBP-FRB, and the gene editing dimerization molecule is small molecule, e.g., rapamycin or a rapalog, e.g., RAD001. In some aspects, the gene editing switch domain is a mutant FKBP domain, e.g., as described herein. In some aspects, the gene editing switch domain is a mutant FRB domain, e.g., as described herein. In some aspects, the gene editing switch domain is a polypeptide-based moiety, e.g., an scFv that binds a myc peptide, and the gene editing dimerization molecule is a polypeptide, a fragment thereof, or a multimer of a polypeptide, e.g., a myc ligand or multimers of a myc ligand that bind to one or more myc scFvs. In some aspects, the gene editing switch domain is a polypeptide-based moiety, e.g., myc receptor, and the gene editing dimerization molecule is an antibody or fragment thereof, e.g., myc antibody.

The term “FRB fragment or analog thereof” as used herein, refers to a FRB derived polypeptide that, in the presence of a dimerization molecule, e.g., rapamycin or a rapamycin analog, binds to or associates with a FKBP and/or a FKBP fragment or analog thereof, or a complex formed between a FKBP and/or a FKBP fragment or analog thereof and a dimerization molecule, and/or has the ability to form a complex with between a FKBP and/or a FKBP fragment or analog thereof and a dimerization molecule. In some aspects the FRB fragment or analog thereof is FRB, e.g., SEQ ID NO: 2 (NCBI GenBank accession number NP 004949.1, amino acid residues 2021 to 2112). In some aspects the FRB fragment or analog thereof comprises one or more, e.g., 2, 3, 4, 5, 6, 7, 8, 9, 10 or more, mutations in the amino acid sequence of a wild-type FRB, e.g., a FRB comprising SEQ ID NO: 2. In some aspects the FRB fragment or analog thereof comprises 30, 35, 40, 45, 50, 55, 60, 65, 70, 75, 80, 85, or 90 amino acids of the sequence of FRB, e.g., SEQ ID NO: 2. In some aspects the FRB fragment or analog thereof comprises at least 70, 75, 80, 85, 90, 95, 96, 97, 98 or 99% identity with SEQ ID NO: 2. In some aspects, the FRB fragment or analog thereof comprises one or more mutations which increase the affinity of binding with rapamycin or a rapamycin analog, e.g., RAD001, or a mutation described in the section herein entitled FRB MUTANTS. In some aspects, the FRB fragment or analog thereof comprises: an E2032 mutation, e.g., an E2032I mutation or E2032L mutation; a T2098 mutation, e.g., a T2098L mutation; or an E2032 and a T2098 mutation, e.g., an E2032I and a T2098L or an E2032L and a T2098L mutation.

As the term is used herein, “FKBP fragment or analog thereof” refers to a FKBP derived polypeptide that, in the presence of a dimerization molecule, e.g., rapamycin or a rapamycin analog, binds to or associates with FRB and/or a FRB fragment or analog thereof, or a complex formed between the FRB and/or a FRB fragment or analog thereof and the dimerization molecule, and/or has the ability to form a complex with between a FRB and/or a FRB fragment or analog thereof and a dimerization molecule. In some aspects the FKBP fragment or analog thereof is FKBP, e.g., SEQ ID NO: 1 or 3. SEQ ID NO: 3 corresponds to NCBI GenBank accession number NP_000792.1. In some aspects the FKBP fragment or analog thereof comprises one or more, e.g., 2, 3, 4, 5, 6, 7, 8, 9, 10 or more, mutations in the amino acid sequence of a wild-type FKBP, e.g., a FKBP comprising SEQ ID NO: 1 or 3. In some aspects the FKBP fragment or analog thereof comprises 30, 35, 40, 45, 50, 55, 60, 65, 70, 75, 80, 85, or 90 amino acids of the sequence of FKBP, e.g., SEQ ID NO: 1 or 3. In some aspects the FKBP fragment or analog thereof comprises at least 70, 75, 80, 85, 90, 95, 96, 97, 98 or 99% identity with SEQ ID NO: 1 or 3. In some aspects, the FKBP fragment or analog thereof comprises one or more mutations which enhances the formation of a complex between the FKBP fragment or analog thereof, a FRB fragment or analog thereof, and the dimerization molecule, rapamycin, or a rapalog, e.g., RAD001, or a mutation described in the section herein entitled FKBP MUTANTS. In some aspects, the FKBP fragment or analog thereof comprises a mutation at one or more amino acid positions(s) selected from a tyrosine at position 26 (Y26), phenylalanine at position 36 (F36), aspartic acid at position 37 (D37), arginine at position 42 (R42), lysine at position 44 (K44), proline at position 45 (P45), phenylalanine at position 46 (F46), glutamine at position 53 (Q53), glutamic acid at position 54 (E54), valine at position 55 (V55), isoleucine at position 56 (156), tryptophan at position 59 (W59), tyrosine at position 82 (Y82), histidine at position 87 (H87), glycine at position 89 (G89), isoleucine at position 90 (I90), isoleucine at position 91 (I91) and phenylalanine at 99 (F99), where Y26, F36, D37, R42, K44, P45, F46, Q53, E54, V55, I56, W59, Y82, H87, G89, I90, I91 and F99 is mutated to any other naturally-occurring amino acid.

The terms “modulate” or “modulating” as used herein in connection with gene expression refers to altering of the level of expression of the gene relative to any baseline level of expression. Modulating gene expression can include, for example, repression of expression or upregulation of expression. Modulation can be mediated, for example, at the transcription level, the translation level, or at the post-translation level. Levels of expression of a gene can be quantified to determine if expression has been modulated by any quantitative method known in the art, e.g., quantitiative PCR or quantitative binding assay.

The terms “modify” or “modifying” as used herein in connection with an endogenous nucleic acid sequence refers to the chemical alteration of the target nucleic acid sequence. In one aspect, the modifying comprises breaking a covalent bond present in the target nucleic acid sequence, e.g., a covalent bond of the target nucleic acid phosphodiester backbone. In one aspect, the modifying comprises the removal or excision of one or more base pairs from the target nucleic acid sequence. In one aspect, the modifying comprises the addition of one or more base pairs to the target nucleic acid sequence. The modifying may occur in one step or in more than one step.

### Description

The present invention provides gene editing systems comprising gene editing dimerization switches that allow for the regulation of a gene editing function by the introduction, e.g., administration, of a gene editing dimerization molecule. A regulated gene editing function provides, e.g., less off-target side effects, and increases the therapeutic window.

The present invention also provides improved FKBP/FRB-based dimerization switches wherein the FRB switch domain or the FKBP switch domain, or both the FRB and FKBP switch domains, comprise one or more mutations that optimize performance, e.g., that alter, e.g., enhance the formation of a complex between the first switch domain, the second switch domain, and the dimerization molecule, rapamycin, or a rapalog, e.g., RAD001.

Without wishing to be bound by theory, it is believed that enhancing the formation of a complex between an FRB-derived switch domain, a FKBP-derived switch domain, and a dimerization molecule, rapamycin, or a rapalog, e.g., RAD001 can optimize the response of the switch to a dimerization molecule, and, e.g., allow the use of lower concentrations of the dimerization molecule to dimerize heterologous domains bound to the switch domains. Some dimerization molecules induce immunosuppressive effects at certain dosages, and therefore have limited use in vivo. Thus, the ability to use lower concentrations of the dimerization molecule can increase the range of dosages of dimerization molecule that can be used without inducing immunosuppression. Alternatively or in addition, without wishing to be bound by theory, it is believed that use of mutant FRB switch domain that enhances the formation of a complex between the mutant FRB switch domain, a FKBP-derived switch domain, and the dimerization molecule, rapamycin, or a rapalog, e.g., RAD001, can result in preferential binding of the dimerization molecule to the mutant FRB instead of binding and inhibiting endogenous FRAP/mTOR. Preventing the inhibition of endogenous FRAP/mTOR decreases or inhibits adverse effects associated with endogenous FRAP/mTOR inhibition, e.g., toxicity or immunosuppression.

FKBP/FRAP

FKBP12 (FKBP, or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR), thereby acting to dimerize these molecules.

FKPB/FRAP Based Switches

FKBP Derived Switch Domains

The sequences of FKBP is as follows:|

(SEQ ID NO: 1)
D V P D Y A S L G G P S S P K K K R K V S R G V Q
V E T I S P G D G R T F P K R G Q T V V H Y T G M
L E D G K K F D S S R D R N K P F K F M L G K Q E
V I R G W E E G V A Q M S V G Q R A K L T I S P D
Y A Y G A T G H P G I I P P H A T L V F D V E L L
K L E T S Y

In some aspects, an FKBP switch domain can comprise a FRB binding fragment of

FKBP or a FKBP analog, e.g., the underlined portion of SEQ ID NO 1, which is:

(NCBI Genebank accession number NP_000792.1)
(SEQ ID NO: 3)
G V Q V E T I S P G D G R T F P K R G Q T C V V H
Y T G M L E D G K K F D S S R D R N K P F K F M L
G K Q E V I R G W E E G V A Q M S V G Q R A K L T
I S P D Y A Y G A T G H P G I I P P H A T L V F D
V E L L K L E.

In an aspect, a FRB binding fragment of FKBP or a FKBP analog comprises 30, 35, 40, 45, 50, 55, 60, 70, 75, 80, 85 or 90 amino acids of the sequence of FKBP, SEQ ID NO:1 or SEQ ID NO: 3, and, in some aspects, further comprises one or a plurality, e.g., 2, 3, 4, or 5, mutations that optimize binding, e.g., one or a plurality, e.g., 2, 3, 4, or 5, mutations described herein. In an aspect, the FRB binding fragment of FKBP or a FKBP analog is at least 5, 10, 15, 20, 25, 30, 35, 40 amino acids shorter than the sequence of FKBP, SEQ ID NO:1 or SEQ ID NO: 3, and, in some aspects, further comprises one or a plurality, e.g., 2, 3, 4, or 5, mutations that optimize binding, e.g., one or a plurality, e.g., 2, 3, 4, or 5, mutations described herein.

In an aspect, the FRB binding fragment of FKBP or FKBP analog comprises: at least 70, 75, 80, 85, 90, 95, 96, 97, 98, or 99% identity with, or differs by no more than 30, 25, 20, 15, 10, 5, 4, 3, 2, or 1 amino acid residues from, the FKBP sequence of SEQ ID NO: 1 or SEQ ID NO: 3, and, in some aspects, further comprises one or a plurality, e.g., 2, 3, 4, or 5, mutations that optimize binding, e.g., one or a plurality, e.g., 2, 3, 4, or 5, mutations described herein.

FRAP or FRB Derived Switch Domains

FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51).

The sequence of FRB is as follows:

(NCBI Genebank accession number NP_004949.1
(amino acid residues 2021-2113))
(SEQ ID NO: 2)
ILWHEMWHEG LEEASRLYFG ERNVKGMFEV LEPLHAMMER
GPQTLKETSF NQAYGRDLME AQEWCRKYMK SGNVKDLTQA
WDLYYHVFRR ISK.

In an aspect, a FKBP binding fragment of FRB or FRB analog comprises 30, 35, 40, 45, 50, 55, 60, 70, 75, 80, 85 or 90 amino acids of the sequence of FRB, SEQ ID NO:2, and, in some aspects, further comprises one or a plurality, e.g., 2, 3, 4, or 5, mutations that optimize binding, e.g., one or a plurality, e.g., 2, 3, 4, or 5, mutations described herein. In an aspect, the FKBP binding fragment of FRB or FRB analog is at least 5, 10, 15, 20, 25, 30, 35, 40 amino acids shorter than the sequence of FRB, SEQ ID NO:2, and, in some aspects, further comprises one or a plurality, e.g., 2, 3, 4, or 5, mutations that optimize binding, e.g., one or a plurality, e.g., 2, 3, 4, or 5, mutations described herein.

In an aspect, the FKBB binding fragment of FRB or FRB analog comprises: at least 70, 75, 80, 85, 90, 95, 96, 97, 98, or 99% identity with, or differs by no more than 30, 25, 20, 15, 10, 5, 4, 3, 2, or 1 amino acid residues from, the FRB sequence of SEQ ID NO: 2, and, in some aspects, further comprises one or a plurality, e.g., 2, 3, 4, or 5, mutations that optimize binding, e.g., one or a plurality, e.g., 2, 3, 4, or 5, mutations described herein.

In an aspect, an FKBP/FRAP, e.g., an FKBP/FRB, based switch comprises one switch domain comprising amino acid residues disclosed in SEQ ID NO: 1, or an FRB binding fragment or FKBP analog, e.g., SEQ ID NO:3, and one switch domain comprises amino acid residues disclosed in SEQ ID NO: 2 or an FKPB binding fragment or FRB analog.

In some aspects, an FKBP/FRAP, e.g., an FKBP/FRB, based switch can use a heterodimerization molecule, e.g., a rapamycin analog, that lacks rapamycin's undesirable properties, e.g., it lacks or has less immunosuppressive activity. Examples of suitable dimerization molecules are described herein.

Mutant Switch Domains

Mutations in the switch domains, e.g., an FRB or FKBP switch domain, that enhance formation of a complex between the FRB switch domain, the FKBP switch domain, and a dimerization molecule, rapamycin, or a rapalog, e.g., RAD001 can be identified using a screening method described herein. First, regions or amino acid residues in a wild-type FRB or FKBP switch domain that are present in the dimerization molecule-binding pocket of the natively folded wild-type FRB or FKBP switch domain, or contribute to the interaction, e.g., directly or indirectly, with the dimerization molecule, can be determined from structural data, e.g., x-ray crystallographic structures, or computer modeling, e.g., homology or comparative modeling of homologous proteins bound to the dimerization molecule or derivatives thereof.

Alternatively, or in addition, mutations in the switch domains that confer enhanced dimerization with the other switch domain in the switch in the presence of a dimerization molecule can also be identified using a screening method described herein. The amino acids of one switch domain, e.g., FRB switch domain, that contribute to interacting with the second switch domain, e.g., FKBP switch domain, in the presence of the dimerization molecule, can also be mutated to confer increased dimerization activity between the switch domains. Dimerization activity, as used herein, can refer to the affinity between the switch domains, or the kinetics, e.g., speed, of dimerization of the switch domains, in the presence of the dimerization molecule

A candidate mutant switch domain that may have altered, e.g., enhanced formation of a complex between the mutant switch domain, a second switch domain, and a dimerization molecule, rapamycin, or a rapalog, e.g., RAD001 can be generated by mutating the target region or target residue that may contribute to the affinity of the switch domain, e.g., FRB or FKBP switch domain, to a dimerization molecule or a complex formed between the dimerization molecule and a switch domain, e.g., by PCR site-directed mutagenesis. In some aspects, an unbiased approach for generating a library of candidate mutant switch domains in which putative sites that confer enhanced formation of a complex between the mutant switch domain, a second switch domain, and a dimerization molecule, rapamycin, or a rapalog, e.g., RAD001 is mutated to all other possible amino acids. In an aspect, a library of candidate mutant switch domains comprising one or more point mutations can be generated using a saturation mutagenesis approach, where a target residue is mutated to all other possible amino acids by randomizing the codon that encodes the target residue by PCR amplification. Randomization of each codon corresponding to a target residue can be achieved by using a codon library that represents all 20 amino acids, e.g., a NNK library, where N can be adenine (A), cytosine (C), guanine (G), or thymine (T), and K can be guanine (G) or thymine (T). Table 1 shows the codon distribution of an exemplary NNK library and the corresponding amino acids. Each codon in the NNK library is incorporated at the target residue position, thereby producing a library of candidate mutant switch domains for each target residue position where the target residue position has been mutated to every other possible amino acid. The library of candidate FRB mutants can then be screened to identify candidate mutant switch domains which enhance formation of a complex between the FRB mutant derived switch domain, a second switch domain, and a dimerization molecule, rapamycin, or a rapalog, e.g., RAD001.

TABLE 1
NNK Library
DNA base N defined to be A/C/G/T and K defined to be G/T.

| NNK | Amino Acid |
|---|---|
| AAG | Lysine, Lys, K |
| AAT | Asparagine, Asn, N |
| ACG | Theronine, Thr, T |
| ACT | Theronine, Thr, T |
| AGG | Arginine, Arg, R |
| AGT | Serine, Ser, S |
| ATG | Methionine, Met, M |
| ATT | Isoleucine, Ile, I |
| CAG | Glutamine, Gln, Q |
| CAT | Histidine, His, H |
| CCG | Proline, Pro, P |
| CCT | Proline, Pro, P |
| CGG | Arginine, Arg, R |
| CGT | Arginine, Arg, R |
| CTG | Leucine, Leu, L |
| CTT | Leucine, Leu, L |
| GAG | Glutamic acid, Glu, E |
| GAT | Aspartic acid, Asp, D |
| GCG | Alanine, Ala, A |
| GCT | Alanine, Ala, A |
| GGG | Glycine, Gly, G |
| GGT | Glycine, Gly, G |
| GTG | Valine, Val, V |
| GTT | Valine, Val, V |
| TAG | Stop |
| TAT | Tyrosine, Tyr, Y |
| TCG | Serine, Ser, S |
| TCT | Serine, Ser, S |
| TGG | Tryptophan, Trp, W |
| TGT | Cysteine, Cys, C |
| TTG | Leucine, Leu, L |
| TTT | Phenylalanine, Phe, F |

Candidate mutant switch domains can also be generated by site-specific mutagenesis to a specific amino acid, e.g., a conservative or non-conservative amino acid substitution.

Conservative amino acid substitutions are ones in which the amino acid residue is replaced with an amino acid residue having a similar side chain. In some aspects, a conservative amino acid modification does not result in a substantial change in binding or affinity of the switch domain. In other aspects, a conservative amino acid modification alters, e.g., enhances the formation of a complex between the first switch domain, a second switch domain, and a dimerization molecule, rapamycin, or a rapalog, e.g., RAD001. Substitutions can be introduced into a switch domain described herein by standard techniques known in the art, such as site-directed mutagenesis and PCR-mediated mutagenesis. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Amino acids may also be grouped according to common side-chain size, for example, small amino acids (Gly, Ala, Ser, Pro, Thr, Asp, Asn), or bulky hydrophobic amino acids (Met, He, Leu).

In some aspects, substantial modifications in the biological properties, e.g., binding affinity, of the switch domain can be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will entail exchanging a member of one of the families described above for a member of another family.

Multiple Switch Domains

Aspects of the dimerization switches described herein feature multiple switch domains, sometimes referred to herein as a multi switch. A multi switch comprises a plurality, e.g., 2, 3, 4, 5, 6, 7, 8, 9, or 10, switch domains, independently, on a first and second polypeptide. In an aspect the first polypeptide can comprise a plurality of first switch domains, e.g., FKBP-derived switch domains, and the second polypeptide can comprise a plurality of second switch domains, e.g., FRB-derived switch domains. In an aspect the first polypeptide can comprise a first and a second switch domain, e.g., an FKBP-derived switch domain and an FRB-derived switch domain, and the second polypeptide can comprise a first and a second switch domain, e.g., an FKBP-derived switch domain and an FRB-derived switch domain. In an aspect the first polypeptide can comprise an asymmetrical number of first and second switch domains, and/or the second polypeptide can comprise an asymmetrical number of first and second switch domains. For example, the first polypeptide can comprise one first switch domain, e.g., an FKBP derived switch domain, and more than one, e.g., 2, second switch domains, e.g., FRB derived switch domains; and the second polypeptide can comprise one first switch domain, e.g., a FRB derived switch domain, and more than one, e.g., 2, second switch domains, e.g., FKBP derived switch domains.

Screening Assays

Various screening assays can be used to evaluate candidate mutant switch domains to identify those switch domains which enhance the formation of a complex between the mutant switch domain, the second switch domain, and a dimerization molecule, rapamycin, or a rapalog, e.g., RAD001, or increase dimerization activity with the other switch domain in the presence of the dimerization molecule. Suitable binding assays are known in the art and are further described herein.

In a direct binding assay, unlabeled candidate mutant FRB is incubated in solution with tagged wild-type FKBP in the presence of the dimerization molecule, e.g., under conditions suitable for binding of FRB to the dimerization molecule and dimerization of FRB and FKBP. Tagged FKBP can be removed from the reaction by affinity purification; candidate mutant FRB that is able to bind the dimerization molecule and dimerize with the tagged FKBP will also be removed. The amount of free candidate mutant FRB that does not dimerize with the tagged wild-type FKBP can be calculated by determining protein concentration of the reaction. EC50 values for direct binding affinity can then be calculated using methods known in the art.

Alternatively or in addition to the direct binding assay described above, a competition binding assay can also be performed to identify a mutant FRB which enhances formation of a complex between the FRB mutant derived switch domain, a second switch domain, e.g., a FKBP derived switch domain, and a dimerization molecule, rapamycin, or a rapalog, e.g., RAD001. In this assay, an untagged candidate mutant FRB is incubated in solution with: 1) wild-type FKBP coupled to a first tag, e.g., biotinylated wild-type FKBP; 2) wild-type FRB coupled to a second tag, e.g., FLAG-tagged wild-type FRB; and 3) the dimerization molecule; under conditions suitable for binding of FRB to the dimerization molecule and dimerization of FRB and FKBP. The tagged wild-type FKBP and tagged wild-type FRB can be removed from the reaction by affinity purification. The amount of free candidate mutant FRB that does not dimerize with the tagged wild-type FKBP in the presence of wild-type FRB can be calculated by determining protein concentration of the reaction. EC50 values for competition binding affinity can then be calculated using methods known in the art.

FRB Mutants

In an aspect, a mutant FRB derived switch domain comprises one or more, e.g., 2, 3, 4, 5, 6, 7, 8, 9, 10 or more, mutations in the amino acid sequence of a wild-type FRB, e.g., a FRB comprising SEQ ID NO: 2. The mutant FRB derived switch domain enhances formation of a complex between the mutant FRB derived switch domain, a second switch domain, e.g., a FKBP derived switch domain, and a dimerization molecule, rapamycin, or a rapalog, e.g., RAD001, e.g., as compared to the complex formed with wild-type FRB. The amino acid position numbering of a wild-type or mutant FRB derived switch domain referred to herein can be determined from SEQ ID NO: 2, where the first amino acid of SEQ ID NO: 2 is position 2021 and the last amino acid of SEQ ID NO: 2 is position 2113.

In an aspect, a mutant FRB derived switch domain comprises one or more mutations at an amino acid position(s) selected from: a leucine at position 2031 (L2031), a glutamic acid at position 2032 (E2032), a serine at position 2035 (S2035), an arginine at position 2036 (R2036), a phenylalanine at position 2039(F2039), a glycine at position 2040 (G2040), a threonine at position 2098 (T2098), a tryptophan at position 2101(W2101), an aspartic acid at position 2102(D2102), a tyrosine at position 2105(Y2105), and a phenylalanine at position 2108 (F2108), where L2031, E2032, S2035, R2036, F2039, G2040, T2098, W2101, D2102, Y2105, and/or F2108 is mutated to any other naturally-occurring amino acid.

In an aspect, a mutant FRB derived switch domain comprises an amino acid sequence selected from SEQ ID NOs: 4-14, where X can be any naturally occurring amino acid. Amino acid sequences of exemplary mutant FRB switch domains which enhance formation of a complex between the FRB mutant derived switch domain, a second switch domain, e.g., a FKBP derived switch domain, and a dimerization molecule, rapamycin, or a rapalog, e.g., RAD001 are provided in Table 2 below. A screen, as described herein, can be performed to identify the mutant FRB derived switch domain which enhances formation of a complex between the FRB mutant derived switch domain, a second switch domain, e.g., a FKBP derived switch domain, and a dimerization molecule, rapamycin, or a rapalog, e.g., RAD001.

TABLE 2
Exemplary mutant FRB derived switch domains
which enhance formation of a complex between the
FRB mutant derived switch domain, a second
switch domain, e.g., a FKBP derived switch domain,
and a dimerization molecule, rapamycin,
or a rapalog, e.g., RAD001.

| | | SEQ |
|---|---|---|
| | | ID |
| FRB mutant | Amino Acid Sequence | NO: |
| L2031 mutant | ILWHEMWHEGXEEASRLYFGERNVKGM | 4 |
| | FEVLEPLHAMERGPQTLKETSFNQAYG | |
| | RDLMEAQEWCRKYMKSGNVKDLTQAWD | |
| | LYYHVFRRISK | |
| E2032 mutant | ILWHEMWHEGLXEASRLYFGERNVKGM | 5 |
| | FEVLEPLHAMERGPQTLKETSFNQAYG | |
| | RDLMEAQEWCRKYMKSGNVKDLTQAWD | |
| | LYYHVFRRISK | |
| S2035 mutant | ILWHEMWHEGLEEAXRLYFGERNVKGM | 6 |
| | FEVLEPLHAMERGPQTLKETSFNQAYG | |
| | RDLMEAQEWCRKYMKSGNVKDLTQAWD | |
| | LYYHVFRRISK | |
| R2036 mutant | ILWHEMWHEGLEEASXLYFGERNVKGM | 7 |
| | FEVLEPLHAMERGPQTLKETSFNQAYG | |
| | RDLMEAQEWCRKYMKSGNVKDLTQAWD | |
| | LYYHVFRRISK | |
| F2039 mutant | ILWHEMWHEGLEEASRLYXGERNVKGM | 8 |
| | FEVLEPLHAMERGPQTLKETSFNQAYG | |
| | RDLMEAQEWCRKYMKSGNVKDLTQAWD | |
| | LYYHVFRRISK | |
| G2040 mutant | ILWHEMWHEGLEEASRLYFXERNVKGM | 9 |
| | FEVLEPLHAMERGPQTLKETSFNQAYG | |
| | RDLMEAQEWCRKYMKSGNVKDLTQAWD | |
| | LYYHVFRRISK | |
| T2098 mutant | ILWHEMWHEGLEEASRLYFGERNVKGM | 10 |
| | FEVLEPLHAMERGPQTLKETSFNQAYG | |
| | RDLMEAQEWCRKYMKSGNVKDLXQAWD | |
| | LYYHVFRRISK | |
| W2101 mutant | ILWHEMWHEGLEEASRLYFGERNVKGM | 11 |
| | FEVLEPLHAMERGPQTLKETSFNQAYG | |
| | RDLMEAQEWCRKYMKSGNVKDLTQAXD | |
| | LYYHVFRRISK | |
| D2102 mutant | ILWHEMWHEGLEEASRLYFGERNVKGM | 12 |
| | FEVLEPLHAMERGPQTLKETSFNQAYG | |
| | RDLMEAQEWCRKYMKSGNVKDLTQAWX | |
| | LYYHVFRRISK | |
| Y2105 mutant | ILWHEMWHEGLEEASRLYFGERNVKGM | 13 |
| | FEVLEPLHAMERGPQTLKETSFNQAYG | |
| | RDLMEAQEWCRKYMKSGNVKDLTQAWD | |
| | LYXHVFRRISK | |
| F2108 mutant | ILWHEMWHEGLEEASRLYFGERNVKGM | 14 |
| | FEVLEPLHAMERGPQTLKETSFNQAYG | |
| | RDLMEAQEWCRKYMKSGNVKDLTQAWD | |
| | LYYHVXRRISK | |

A screen can be performed to evaluate candidate mutant FRB derived switch domains which enhance formation of a complex between the FRB mutant derived switch domain, a second switch domain, e.g., a FKBP derived switch domain, and a dimerization molecule, rapamycin, or a rapalog, e.g., RAD001, as further described in herein and in Examples 1 and 2.

In an aspect, a mutant FRB derived switch domain, e.g., which enhances formation of a complex between the FRB mutant derived switch domain, a second switch domain, e.g., a FKBP derived switch domain, and a dimerization molecule, rapamycin, or a rapalog, e.g., RAD001, comprises one or more mutations at the amino acid(s) selected from L2031, E2032, S2035, R2036, F2039, G2040, T2098, W2101, D2102, Y2105, and F2108, where the wild-type amino acid is mutated to any other naturally-occurring amino acid. In an aspect, a mutant FRB derived switch domain comprises a mutation at E2032, where E2032 is mutated to phenylalanine (E2032F), methionine (E2032M), arginine (E2032R), valine (E2032V), tyrosine (E2032Y), isoleucine (E2032I), e.g., SEQ ID NO: 15, or leucine (E2032L), e.g., SEQ ID NO: 16.

In an aspect, a mutant FRB derived switch domain comprises a mutation at T2098, where T2098 is mutated to phenylalanine (T2098F) or leucine (T2098L), e.g., SEQ ID NO: 17.

In an aspect, a mutant FRB derived switch domain comprises a mutation at E2032 and at T2098, where E2032 is mutated to any amino acid other than E, and where T2098 is mutated to any amino acid other than T, e.g., SEQ ID NO: 18. In an aspect, a mutant FRB derived switch domain comprises an E2032I and a T2098L mutation, e.g., SEQ ID NO: 19. In an aspect, a mutant FRB derived switch domain comprises an E2032L and a T2098L mutation, e.g., SEQ ID NO: 20. In some aspects, the mutant FRB derived switch domain comprises a mutation at E2032, e.g., E2032I or E2032L, and/or a mutation at T2098, e.g., T2098L, and a combination with one or more of any of the other mutations described herein, e.g., L2031, S2035, R2036, F2039, G2040, W2101, D2102, Y2105, and F2108.

Amino acid sequences of exemplary mutant FRB derived switch domains which enhance formation of a complex between the FRB mutant derived switch domain, a second switch domain, e.g., a FKBP derived switch domain, and a dimerization molecule, rapamycin, or a rapalog, e.g., RAD001 are provided in Table 3.

TABLE 3
Exemplary mutant FRB derived switch domains
which enhance formation of a complex between the
FRB mutant derived switch domain, a second switch
domain, e.g., a FKBP derived switch domain, and a
dimerization molecule, rapamycin, or a rapalog,
e.g., RAD001.

| | | SEQ |
|---|---|---|
| | | ID |
| FRB mutant | Amino Acid Sequence | NO: |
| E2032I mutant | ILWHEMWHEGLIEASRLYFGERNVKGM | 15 |
| | FEVLEPLHAMERGPQTLKETSFNQAYG | |
| | RDLMEAQEWCRKYMKSGNVKDLTQAWD | |
| | LYYHVFRRISK | |
| E2032L mutant | ILWHEMWHEGLLEASRLYFGERNVKGM | 16 |
| | FEVLEPLHAMERGPQTLKETSFNQAYG | |
| | RDLMEAQEWCRKYMKSGNVKDLTQAWD | |
| | LYYHVFRRISK | |
| T2098L mutant | ILWHEMWHEGLEEASRLYFGERNVKGM | 17 |
| | FEVLEPLHAMERGPQTLKETSFNQAYG | |
| | RDLMEAQEWCRKYMKSGNVKDLLQAWD | |
| | LYYHVFRRISK | |
| E2032X, T2098X | ILWHEMWHEGLXEASRLYFGERNVKGM | 18 |
| mutant | FEVLEPLHAMERGPQTLKETSFNQAYG | |
| | RDLMEAQEWCRKYMKSGNVKDLXQAWD | |
| | LYYHVFRRISK | |
| E2032I, T2098L | ILWHEMWHEGLIEASRLYFGERNVKGM | 19 |
| mutant | FEVLEPLHAMMERGPQTLKETSFNQAY | |
| | GRDLMEAQEWCRKYMKSGNVKDLLQAW | |
| | DLYYHVFRRISK | |
| E2032L, T2098L | ILWHEMWHEGLLEASRLYFGERNVKGM | 20 |
| mutant | FEVLEPLHAMMERGPQTLKETSFNQAY | |
| | GRDLMEAQEWCRKYMKSGNVKDLLQAW | |
| | DLYYHVFRRISK | |

FKBP Mutants

In an aspect, a mutant FKBP derived switch domain comprises one or more, e.g., 2, 3, 4, 5, 6, 7, 8, 9, 10 or more, mutations in the amino acid sequence of a wild-type FKBP, e.g., a FKBP comprising SEQ ID NO: 2. The mutant FKBP derived switch domain comprises increased affinity for a dimerization molecule, e.g., as compared to the affinity of wild-type FKBP for the dimerization molecule and/or comprise enhanced formation of a complex between the mutant FKBP derived switch domain, a second switch domain, e.g., a FRB derived switch domain, and a dimerization molecule, rapamycin, or a rapalog, e.g., RAD001. The amino acid position numbering of a wild-type or mutant FKBP derived switch domain referred to herein can be determined from SEQ ID NO: 1 or 3, where the first amino acid of SEQ ID NO: 3 is position 1 and the last amino acid of SEQ ID NO: 3 is position 108.

In an aspect, a mutant FKBP derived switch domain comprises a mutation at one or more amino acid positions(s) selected from a tyrosine at position 26 (Y26), phenylalanine at position 36 (F36), aspartic acid at position 37 (D37), arginine at position 42 (R42), lysine at position 44 (K44), proline at position 45 (P45) phenylalanine at position 46 (F46), glutamine at position 53 (Q53), glutamic acid at position 54 (E54), valine at position 55 (V55), isoleucine at position 56 (I56), tryptophan at position 59 (W59), tyrosine at position 82 (Y82), histidine at position 87 (H87), glycine at position 89 (G89), isoleucine at position 90 (I90), isoleucine at position 91 (I91), and phenylalanine at 99 (F99), where Y26, F36, D37, R42, K44, P45, F46, Q53, E54, V55, I56, W59, Y82, H87, G89, I90, I91, and F99 is mutated to any other naturally-occurring amino acid. In an aspect, a mutant FKBP derived switch domain comprises a mutation at one or more of Y26, F36, D37, R42, F46, Q53, E54, V55, I56, W59, Y82, H87, G89, I90, and F99. In an aspect, a mutant FKBP derived switch domain comprises a mutation at one or more of Q53, R42, I56, W59, Y82, G89, I90 or H87.

In an aspect, a mutant FKBP derived switch domain comprises an amino acid sequence selected from SEQ ID NOs: 21-35, where X can be any naturally occurring amino acid other than the amino acid in the corresponding position of SEQ ID NO: 3.

TABLE 4
Exemplary mutant FKBP derived switch domains having
increased affinity for a FKBP/FRB derived switch
dimerization molecule and/or which enhance
formation of a complex between the mutant FKBP
derived switch domain, a second switch
domain, e.g., a FRB derived switch domain, and
a dimerization molecule, rapamycin, or a rapalog,
e.g., RAD001.

| | | SEQ |
|---|---|---|
| | | ID |
| Name | Amino Acid Sequence | NO: |
| FKBP Y26 library | GVQVETISPGDGRTFPKRGQTCVVHXT | 21 |
| | GMLEDGKKFDSSRDRNKPFKFMLGKQE | |
| | VIRGWEEGVAQMSVGQRAKLTISPDYA | |
| | YGATGHPGIIPPHATLVFDVELLKLE | |
| FKBP F36 library | GSGVQVETISPGDGRTFPKRGQTCVVH | 22 |
| | YTGMLEDGKKXDSSRDRNKPFKFMLGK | |
| | QEVIRGWEEGVAQMSVGQRAKLTISPD | |
| | YAYGATGHPGIIPPHATLVFDVELLKL | |
| | E | |
| FKBP D37 library | GVQVETISPGDGRTFPKRGQTCVVHYT | 23 |
| | GMLEDGKKFXSSRDRNKPFKFMLGKQE | |
| | VIRGWEEGVAQMSVGQRAKLTISPDYA | |
| | YGATGHPGIIPPHATLVFDVELLKLE | |
| FKBP R42 library | GVQVETISPGDGRTFPKRGQTCVVHYT | 24 |
| | GMLEDGKKFDSSRDXNKPFKFMLGKQE | |
| | VIRGWEEGVAQMSVGQRAKLTISPDYA | |
| | YGATGHPGIIPPHATLVFDVELLKLE | |
| FKBP F46 library | GVQVETISPGDGRTFPKRGQTCVVHYT | 25 |
| | GMLEDGKKFDSSRDRNKPXKFMLGKQE | |
| | VIRGWEEGVAQMSVGQRAKLTISPDYA | |
| | YGATGHPGIIPPHATLVFDVELLKLE | |
| FKBP Q53 library | GVQVETISPGDGRTFPKRGQTCVVHYT | 26 |
| | GMLEDGKKFDSSRDRNKPFKFMLGKXE | |
| | VIRGWEEGVAQMSVGQRAKLTISPDYA | |
| | YGATGHPGIIPPHATLVFDVELLKLE | |
| FKBP E54 library | GVQVETISPGDGRTFPKRGQTCVVHYT | 27 |
| | GMLEDGKKFDSSRDRNKPFKFMLGKQX | |
| | VIRGWEEGVAQMSVGQRAKLTISPDYA | |
| | YGATGHPGIIPPHATLVFDVELLKLE | |
| FKBP V55 library | GVQVETISPGDGRTFPKRGQTCVVHYT | 28 |
| | GMLEDGKKFDSSRDRNKPFKFMLGKQE | |
| | XIRGWEEGVAQMSVGQRAKLTISPDYA | |
| | YGATGHPGIIPPHATLVFDVELLKLE | |
| FKBP I56 library | GVQVETISPGDGRTFPKRGQTCVVHYT | 29 |
| | GMLEDGKKFDSSRDRNKPFKFMLGKQE | |
| | VXRGWEEGVAQMSVGQRAKLTISPDYA | |
| | YGATGHPGIIPPHATLVFDVELLKLE | |
| FKBP W59 library | GVQVETISPGDGRTFPKRGQTCVVHYT | 30 |
| | GMLEDGKKFDSSRDRNKPFKFMLGKQE | |
| | VIRGXEEGVAQMSVGQRAKLTISPDYA | |
| | YGATGHPGIIPPHATLVFDVELLKLE | |
| FKBP Y82 library | GVQVETISPGDGRTFPKRGQTCVVHYT | 31 |
| | GMLEDGKKFDSSRDRNKPFKFMLGKQE | |
| | VIRGWEEGVAQMSVGQRAKLTISPDYA | |
| | XGATGHPGIIPPHATLVFDVELLKLE | |
| FKBP H87 library | GVQVETISPGDGRTFPKRGQTCVVHYT | 32 |
| | GMLEDGKKFDSSRDRNKPFKFMLGKQE | |
| | VIRGWEEGVAQMSVGQRAKLTISPDYA | |
| | YGATGXPGIIPPHATLVFDVELLKLE | |
| FKBP G89 library | GVQVETISPGDGRTFPKRGQTCVVHYT | 33 |
| | GMLEDGKKFDSSRDRNKPFKFMLGKQE | |
| | VIRGWEEGVAQMSVGQRAKLTISPDYA | |
| | YGATGHPXIIPPHATLVFDVELLKLE | |
| FKBP I90 library | GVQVETISPGDGRTFPKRGQTCVVHYT | 34 |
| | GMLEDGKKFDSSRDRNKPFKFMLGKQE | |
| | VIRGWEEGVAQMSVGQRAKLTISPDYA | |
| | YGATGHPGXIPPHATLVFDVELLKLE | |
| FKBP F99 library | GVQVETISPGDGRTFPKRGQTCVVHYT | 35 |
| | GMLEDGKKFDSSRDRNKPFKFMLGKQE | |
| | VIRGWEEGVAQMSVGQRAKLTISPDYA | |
| | YGATGHPGIIPPHATLVXDVELLKLE | |

A screen, as described herein, can be performed to identify a mutant FKBP derived switch domain having increased affinity for a dimerization molecule for a FKBP-FRB based switch, e.g., rapamycin or a rapalog described herein and/or which enhance formation of a complex between the mutant FKBP derived switch domain, a second switch domain, e.g., a FRB derived switch domain, and a dimerization molecule, rapamycin, or a rapalog, e.g., RAD001. For example, screen can be performed to evaluate a candidate mutant FKBP derived switch domain for increased affinity for the rapalog RAD001 and/or for enhanced formation of a complex between the mutant FKBP derived switch domain, a second switch domain, e.g., a FRB derived switch domain, and RAD001, as further described in herein and in Example 3.

In an aspect, a mutant FKBP derived switch domain, e.g., comprising increased affinity for RAD001 and/or comprising enhanced formation of a complex between the mutant FKBP derived switch domain, a second switch domain, e.g., a FRB derived switch domain, and a dimerization molecule, rapamycin, or a rapalog, e.g., RAD001, comprises one or more mutations at the amino acid(s) selected from Y26, F36, D37, R42, F46, Q53, E54, V55, I56, W59, Y82, H87, G89, I90, and F99, where the wild-type amino acid is mutated to any other naturally-occurring amino acid. In an aspect, a mutant FKBP derived switch domain comprises a mutation at Q53, where Q53 is mutated to threonine (Q53T), or valine (Q53V). In an aspect, a mutant FKBP derived switch domain comprises a mutation at E54, where E54 is mutated to histidine (E54H), lysine (E54K), arginine (E54R), valine (E54V), or tryptophan (E54W). In an aspect, a mutant FKBP derived switch domain comprises a mutation at V55, where V55 is mutated to methionine (V55M) or aspartic acid (V55D). In an aspect, a mutant FKBP derived switch domain comprises a mutation at T85, where T85 is mutated to aspartic acid (T85D) or glutamic acid (T85E).

### Dimerization Molecules

Rapamycin and rapamycin analogs (sometimes referred to as rapalogs), can be used as dimerization molecules in FKBP-FRB based dimerization switches. In an aspect the dimerization molecule can be selected from rapamycin (sirolimus), RAD001 (everolimus), zotarolimus, temsirolimus, AP-23573 (ridaforolimus), biolimus and AP21967.

Rapamycin is a known macrolide antibiotic produced by Streptomyces hygroscopicus having the structure shown in Formula A.

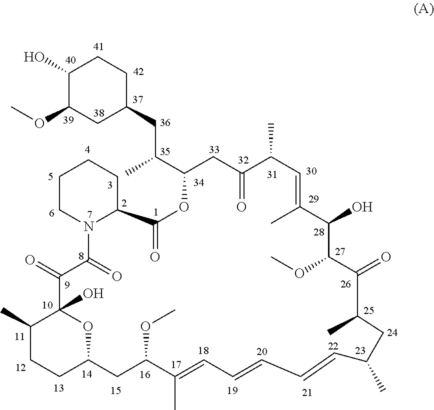 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

See, e.g., McAlpine, J. B., et al., J. Antibiotics (1991) 44:688; Schreiber, S. L., et al., J. Am. Chem. Soc. (1991) 113:7433; U.S. Pat. No. 3,929,992. There are various numbering schemes proposed for rapamycin. To avoid confusion, when specific rapamycin analogs are named herein, the names are given with reference to rapamycin using the numbering scheme of formula A.

Numerous rapamycin analogs can be used as a heterodimerization molecule in a FKBP/FRAP-based dimerization switch. For example, 0-substituted analogues in which the hydroxyl group on the cyclohexyl ring of rapamycin is replaced by OR₁ in which R₁ is hydroxyalkyl, hydroxyalkoxyalkyl, acylaminoalkyl, or aminoalkyl; e.g. RAD001, also known as, everolimus as described in U.S. Pat. No. 5,665,772 and WO94/09010 the contents of which are incorporated by reference. Other suitable rapamycin analogs include those substituted at the 26- or 28-position. The rapamycin analog may be an epimer of an analog mentioned above, particularly an epimer of an analog substituted in position 40, 28 or 26, and may optionally be further hydrogenated, e.g. as described in U.S. Pat. No. 6,015,815, WO95/14023 and WO99/15530 the contents of which are incorporated by reference, e.g. ABT578 also known as zotarolimus or a rapamycin analog described in U.S. Pat. No. 7,091,213, WO98/02441 and WO01/14387 the contents of which are incorporated by reference, e.g. AP23573 also known as ridaforolimus.

Examples of rapamycin analogs suitable for use in the present invention from U.S. Pat. No. 5,665,772 include, but are not limited to, 40-O-benzyl-rapamycin, 40-O-(4′-hydroxymethyl)benzyl-rapamycin, 40-O-[4′-(1,2-dihydroxyethyl)]benzyl-rapamycin, 40-O-allyl-rapamycin, 40-O-[3′-(2,2-dimethyl-1,3-dioxolan-4(S)-yl)-prop-2′-en-1′-yl]-rapamycin, (2′E,4′S)-40-O-(4′,5′-dihydroxypent-2′-en-1′-yl)-rapamycin, 40-O-(2-hydroxy)ethoxycarbonylmethyl-rapamycin, 40-O-(2-hydroxy)ethyl-rapamycin, 40-O-(3-hydroxy)propyl-rapamycin, 40-O-(6-hydroxy)hexyl-rapamycin, 40-O-[2-(2-hydroxy)ethoxy]ethyl-rapamycin, 40-O-[(3S)-2,2-dimethyldioxolan-3-yl]methyl-rapamycin, 40-O-[(2S)-2,3-dihydroxyprop-1-yl]-rapamycin, 40-O-(2-acetoxy)ethyl-rapamycin, 40-O-(2-nicotinoyloxy)ethyl-rapamycin, 40-O-[2-(N-morpholino)acetoxy]ethyl-rapamycin, 40-O-(2-N-imidazolylacetoxy)ethyl-rapamycin, 40-O-[2-(N-methyl-N′-piperazinyl)acetoxy]ethyl-rapamycin, 39-O-desmethyl-39,40-O,O-ethylene-rapamycin, (26R)-26-dihydro-40-O-(2-hydroxy)ethyl-rapamycin, 40-O-(2-aminoethyl)-rapamycin, 40-O-(2-acetaminoethyl)-rapamycin, 40-O-(2-nicotinamidoethyl)-rapamycin, 40-O-(2-(N-methyl-imidazo-2′-ylcarbethoxamido)ethyl)-rapamycin, 40-O-(2-ethoxycarbonylaminoethyl)-rapamycin, 40-O-(2-tolylsulfonamidoethyl)-rapamycin and 40-O-[2-(4′,5′-dicarboethoxy-1′,2′,3′-triazol-1′-yl)-ethyl]-rapamycin.

Other examples of rapamycin analogs where the hydroxyl group on the cyclohexyl ring of rapamycin and/or the hydroxy group at the 28 position is replaced with an hydroxyester group are known, for example, rapamycin analogs found in U.S. RE44,768, e.g. temsirolimus.

Other rapamycin analogs include those wherein the methoxy group at the 16 position is replaced with another substituent, preferably (optionally hydroxy-substituted) alkynyloxy, benzyl, orthomethoxybenzyl or chlorobenzyl and/or wherein the methoxy group at the 39 position is deleted together with the 39 carbon so that the cyclohexyl ring of rapamycin becomes a cyclopentyl ring lacking the 39 position methyoxy group; e.g. as described in WO95/16691 and WO96/41807 the contents of which are incorporated by reference. The analogs can be further modified such that the hydroxy at the 40-position of rapamycin is alkylated and/or the 32-carbonyl is reduced.

Rapamycin analogs from WO95/16691 include, but are not limited to, 16-demethoxy-16-(pent-2-ynyl)oxy-rapamycin, 16-demethoxy-16-(but-2-ynyl)oxy-rapamycin, 16-demethoxy-16-(propargyl)oxy-rapamycin, 16-demethoxy-16-(4-hydroxy-but-2-ynyl)oxy-rapamycin, 16-demethoxy-16-benzyloxy-40-O-(2-hydroxyethyl)-rapamycin, 16-demethoxy-16-benzyloxy-rapamycin, 16-demethoxy-16-ortho-methoxybenzyl-rapamycin, 16-demethoxy-40-O-(2-methoxyethyl)-16-pent-2-ynyl)oxy-rapamycin, 39-demethoxy-40-desoxy-39-formyl-42-nor-rapamycin, 39-demethoxy-40-desoxy-39-hydroxymethyl-42-nor-rapamycin, 39-demethoxy-40-desoxy-39-carboxy-42-nor-rapamycin, 39-demethoxy-40-desoxy-39-(4-methyl-piperazin-1-yl)carbonyl-42-nor-rapamycin, 39-demethoxy-40-desoxy-39-(morpholin-4-yl)carbonyl-42-nor-rapamycin, 39-demethoxy-40-desoxy-39-[N-methyl, N-(2-pyridin-2-yl-ethyl)]carbamoyl-42-nor-rapamycin and 39-demethoxy-40-desoxy-39-(p-toluenesulfonylhydrazonomethyl)-42-nor-rapamycin.

Rapamycin analogs from WO96/41807 include, but are not limited to, 32-deoxo-rapamycin, 16-O-pent-2-ynyl-32-deoxo-rapamycin, 16-O-pent-2-ynyl-32-deoxo-40-O-(2-hydroxy-ethyl)-rapamycin, 16-O-pent-2-ynyl-32-(S)-dihydro-40-O-(2-hydroxyethyl)-rapamycin, 32(S)-dihydro-40-O-(2-methoxy)ethyl-rapamycin and 32(S)-dihydro-40-O-(2-hydroxyethyl)-rapamycin.

Another suitable rapamycin analog is biolimus as described in US2005/0101624 the contents of which are incorporated by reference.

RAD001, otherwise known as everolimus (Afinitor®), has the chemical name (1R,9S,12S,15R,16E,18R,19R,21R,23S,24E,26E,28E,30S,32S,35R)-1,18-dihydroxy-12-{(1R)-2-[(1 S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9]hexatriaconta-16,24,26,28-tetraene-2,3,10,14,20-pentaone (also known as 40-O-(2-hydroxy)ethyl-rapamycin) and the following chemical structure:

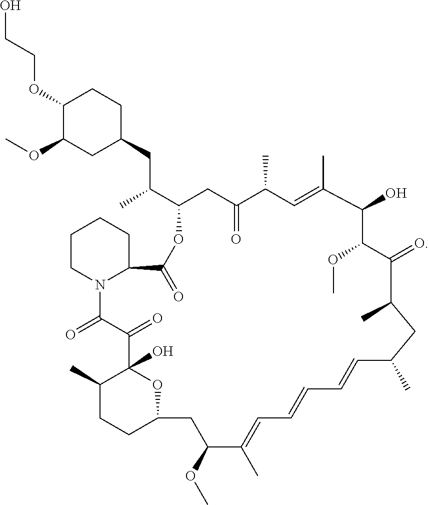 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

### Dimerization Switch-Containing Molecules

The present disclosure features compositions comprising, e.g., a dimerization switch described herein, for use in promoting the association of a moiety coupled to a first switch domain with a moiety coupled to a second switch domain, e.g., modulating, e.g., switching on or off, a biological activity, e.g., in an application described herein, e.g., in the section entitled “Uses of Dimerization Switch-Containing Molecules”. As used herein, the dimerization switch-containing molecules refers to the first moiety coupled, e.g., fused to a first switch domain described herein, or the second moiety coupled, e.g., fused to a second domain described herein, or collectively to both. In any of the aspects described herein, the first switch domain can be a FRB described herein, e.g., a mutant FRB derived switch domain, and the second switch domain can be a FKBP described herein, e.g., a mutant FKBP derived switch domain; or vice versa, where the first switch domain can be a FKBP described herein, e.g., a mutant FKBP derived switch domain, and the second switch domain can be a FRB described herein, e.g., a mutant FRB derived switch domain.

In some aspects, the moiety coupled, e.g., fused, to a first or second switch domain described herein can be a polypeptide. In one aspect, the polypeptide comprises a sequence from an intracellular protein, membrane-bound or a secreted protein. Examples of polypeptides include, but are not limited to: antibodies, growth hormones, cytokines, cytokine receptors, receptor tyrosine kinases, enzymes with post-translational modification activity, detectable proteins (e.g., luciferase, fluorescent proteins), recombinases, or transcription factors, functional fragments thereof. In some aspects there the moiety is a polypeptide, the moiety and the switch domain comprise a fusion protein. Thus, in some aspects, the switch domain and the moiety are encoded by the same nucleic acid.

In an aspect, the moiety coupled, e.g., fused, to a first or second switch domain is not a polypeptide. In an aspect, the moiety coupled, e.g., fused, to a first switch domain is a polypeptide, and the moiety coupled, e.g., fused, to a second switch domain is not a polypeptide. In some aspects where the moiety is not a polypeptide, the moiety comprises a molecule that anchors the switch domain to a membrane, e.g., a myristoyl group or a transmembrane domain. In some aspects, the second switch domain is not coupled or fused to any moiety.

In some aspects, a first or second switch domain described herein is coupled to an moiety, wherein the coupling is a covalent bond or a non-covalent bond. In some aspects, the coupling can be a peptide bond.

In one, the N-terminus of the switch domain is coupled, e.g., fused, to the moiety. In an aspect, the C-terminus of the switch domain is coupled, e.g., fused, to the moiety. In some aspects where more than one moiety is coupled to a switch domain, the switch domain can be disposed between two entities.

In some aspects, a linker is disposed between the switch domain and the moiety. In an aspect, the linker is a peptide sequence comprising 2 to 50 amino acids. For example, the linker may comprise glycine and serine residues. Other linkers, e.g., peptide linker sequences, or small molecule linkers can be used in the art to couple a switch domain to an moiety.

### Uses of Dimerization Switch-Containing Molecules

In one aspect, a dimerization switch described herein is useful in compositions and methods for therapeutic applications, such as treating a subject having a disease, e.g., cancer, or tissue regeneration or repair in a subject.

In another aspect, a dimerization switch described herein is useful in compositions and methods for probing biological mechanisms, e.g., signalling pathways in different biological processes or protein interactions, and the physiological consequences of disrupting such pathways or interactions.

In any of the aspects described herein, a dimerization switch described herein can modulate, e.g., switch on or off, a biological activity. The biological activities modulated by the dimerization switches described herein include: transcriptional regulation, cell proliferation, cell apoptosis, cell differentiation, protein interaction, e.g., association or dissociation, with other proteins, protein translocation, protein stability, e.g., degradation, and protein post-translational modification.

Exemplary uses of a dimerization switch provided in Table 5, and are further described in detail below. Without wishing to be bound by theory, it is believed that the use of dimerization switches, e.g., mutant FRB and/or mutant FKBP switch domains with increased affinity to the dimerization molecule and/or which enhance formation of a complex between a FKBP derived switch domain, a FRB derived switch domain, and a dimerization molecule, rapamycin, or a rapalog, e.g., RAD001, e.g., in the context of exemplary applications described below, increases the dosage range of the dimerization molecule that can be administered, e.g., without inducing immunosuppressive or other adverse effects. Without wishing to be bound by theory, it is believed that with a wider dosage range available, the therapeutic window is increased in vivo, in which lower dosages of dimerization molecule can be used to increase efficacy or biological effect caused by the dimerization, as compared to the efficacy or result achieved with wild-type FKBP/FRB dimerization switches and the maximal dosage of dimerization molecules that do not cause immunosuppressive effects. In any of the uses described in Table 5 and below, the first switch domain can be a FRB described herein, e.g., a mutant FRB derived switch domain, and the second switch domain can be a FKBP described herein, e.g., a mutant FKBP derived switch domain; or vice versa, where the first switch domain can be a FKBP described herein, e.g., a mutant FKBP derived switch domain, and the second switch domain can be a FRB described herein, e.g., a mutant FRB derived switch domain.

TABLE 5
Applications for Dimerization Switches described herein

| | | | |
|---|---|---|---|
| | Moiety coupled to | Moiety coupled to | |
| Use | 1^{st} switch domain | 2^{nd} switch domain | Reference(s) |

Modulation of transcription, e.g, in treating a disease, e.g., cancer

| | | | |
|---|---|---|---|
| Transcriptional | Transactivation domain | DNA binding domain of | Fang, J., et al. (2007). |
| regulation of transgenes, | of a transcription factor, | a transcription factor, | “An antibody delivery |
| e.g., for gene therapy | e.g.: | e.g.,: | system for regulated |
| | C-terminus of NFκB | ZFHD1 DNA binding | expression of |
| | p65 | domain | therapeutic levels of |
| | | | monoclonal antibodies |
| | | | in vivo.” Mol Ther |
| | | | 15(6): 1153-1159; |
| | | | Indraccolo, S., et al. |
| | | | (2006). “Gene therapy |
| | | | of ovarian cancer with |
| | | | IFN-[alpha]-producing |
| | | | fibroblasts: comparison |
| | | | of constitutive and |
| | | | inducible vectors.” Gene |
| | | | Ther 13(12): 953-965; |
| | | | Nguyen, M., et al. |
| | | | (2007). “Rapamycin- |
| | | | regulated control of |
| | | | antiangiogenic tumor |
| | | | therapy following |
| | | | rAAV-mediated gene |
| | | | transfer.” Mol Ther |
| | | | 15(5): 912-920; Rivera, V. M., |
| | | | et al. (1996). “A |
| | | | humanized system for |
| | | | pharmacologic control |
| | | | of gene expression.” Nat |
| | | | Med 2(9): 1028-1032. |

Modulation of signal transduction, e.g., modulation of cell proliferation

| | | | |
|---|---|---|---|
| Regulation of cell | Intracellular signalling | Intracellular signalling | Padrissa-Altes, S., et al. |
| proliferation, e.g., tissue | region of Fgfr4 | region of Fgfr4 | (2014). “Control of |
| repair and regeneration | | | hepatocyte proliferation |
| | | | and survival by Fgf |
| | | | receptors is essential for |
| | | | liver regeneration in |
| | | | mice.” Gut. |
| | FGF2IIIb | FGFRIIIb | Example 3 |

Probing Mechanisms and Function

| | | | |
|---|---|---|---|
| Regulation of protein | Membrane tethering | Akt, e.g., an Akt lacking | Li, B. et al. (2002) “A |
| translocation, e.g., to a | domain, e.g., myristoyl | a PH domain, | novel conditional Akt |
| plasma membrane | group or a | e.g., AktΔPH (deletion | ‘survival switch’ |
| | transmembrane domain | of aas 1-106) | reversibly protects cells |
| | | | from apoptosis.” Gene |
| | | | Ther. 9(4): 233-44; and |
| | | | Li, B., et al. (2007). |
| | | | “Conditional Akt |
| | | | activation promotes |
| | | | androgen-independent |
| | | | progression of prostate |
| | | | cancer.” Carcinogenesis |
| | | | 28(3): 572-583. |
| | Membrane tethering | Fgfr1 intracellular | Welm, B. E., et al. |
| | domain, e.g., myristoyl | signalling domain, e.g., | (2002). “Inducible |
| | group or a | intracellular kinase | dimerization of FGFR1: |
| | transmembrane domain | domain | development of a mouse |
| | | | model to analyze |
| | | | progressive |
| | | | transformation of the |
| | | | mammary gland.” J Cell |
| | | | Biol 157(4): 703-714. |
| Regulation of Cre- | N-terminus of Cre, e.g., | C-terminus of Cre, e.g., | Jullien, N., et al. (2007). |
| mediated recombination | aas 19-59 | 60-343 | « Conditional |
| | | | transgenesis using |
| | | | Dimerizable Cre |
| | | | (DiCre) » PLoS One |
| | | | 2(12): e1355. |
| Imaging protein-protein | N-terminus of luciferase | C-terminus of luciferase | Luker, K. E., et al. |
| interaction | and protein of interest 1; | and potential interactor | (2004). “Kinetics of |
| | N-terminus of luciferase | of protein of interest; or | regulated protein- |
| | and potential interactor | Cterminus of luciferase | protein interactions |
| | of protein of interest | and protein of interest 1 | revealed with firefly |
| | | | luciferase |
| | | | complementation |
| | | | imaging in cells and |
| | | | living animals.” Proc |
| | | | Natl Acad Sci USA |
| | | | 101(33): 12288-12293. |
| | | | Villalobos, V., et al. |
| | | | (2008). “Detection of |
| | | | protein-protein |
| | | | interactions in live cells |
| | | | and animals with split |
| | | | firefly luciferase protein |
| | | | fragment |
| | | | complementation.” |
| | | | Methods Mol Biol 439: |
| | | | 339-352. |

Modulation of the interaction of other components with a moiety fused to a switch domain, e.g.,
modification of the interaction with proteases or other entities that modify or degrated
polypeptides

| | | | |
|---|---|---|---|
| Probing protein function | GSK3b | — | Stankunas, K., et al. |
| | | | (2003). “Conditional |
| | | | protein alleles using |
| | | | knockin mice and a |
| | | | chemical inducer of |
| | | | dimerization.” Mol Cell |
| | | | 12(6): 1615-1624. |
| Regulation of post- | Sumoyltransferase U9 | U9 substrates, e.g., | Zimnik, S., et al. (2009). |
| translational | | STAT1, P53, CRSP9, | “Mutually exclusive |
| modification | | FOS, CSNK2B | STAT1 modifications |
| | | | identified by |
| | | | Ubc9/substrate |
| | | | dimerization-dependent |
| | | | SUMOylation.” Nucleic |
| | | | Acids Res 37(4): e30. |

Nuclear Localization

| | | | |
|---|---|---|---|
| Nuclear Export | NES | Biomolecule to be | Busch, et al., (2009), |
| | | transported out of the | Traffic 10: 1221-1227 |
| | | nucleus | |
| Nuclear Localization | NLS | Polypeptide to be | Busch, et al., (2009), |
| | | transported to the | Traffic 10: 1221-1227; |
| | | nucleus, e.g., a gene | WO2008/0212107; |
| | | editing protein, e.g., a | Urnov (2005) Nature |
| | | zinc finger nuclease, a | 435: 646-651; Cong |
| | | transcription-activator | (2013) Science 339: |
| | | like effector nuclease | 819-823; Huertas, P., |
| | | (TALEN), a Cas9, a | Nat. Struct. Mol. Biol. |
| | | dCas9 or a | (2010) 17: 11-16. |
| | | meganuclease | |

Regulation of Gene Editing Systems

| | Moiety coupled to 1^{st} | Moiety coupled to 2^{nd} | |
|---|---|---|---|
| | gene editing switch | gene editing switch | |
| Use | domain | domain | Reference |
| Regulation of Gene | DNA-binding domain, | DNA-modifying | Cong (2013) Science |
| Editing System | e.g., a zinc finger or | domain, e.g., FokI | 339: 819-823; |
| | engineered zinc finger | | WO2008/0212107; |
| | DNA-binding domain, a | | Urnov (2005) Nature |
| | transcription-activator | | 435: 646-651; Huertas, P., |
| | like effector (TALE) | | Nat. Struct. Mol. |
| | DNA-binding domain, a | | Biol. (2010) 17: 11-16. |
| | Cas9 DNA-binding | | |
| | domain, e.g., dCas9, | | |
| | and/or a CRISPR- | | |
| | associated guide RNA- | | |
| | binding domain | | |

Transcriptional Regulation

In one aspect, a dimerization switch described herein regulates transcription of a transgene. In such aspects, a first switch domain is coupled, e.g., fused, to a transactivation domain of a transcription factor or a functional fragment thereof, and a second switch domain is coupled, e.g., fused, to a DNA binding domain of a transcription factor or a functional fragment thereof. In one aspect, the transactivation domain and the DNA binding domain are from the same transcription factor. In another aspect, the transactivation domain and the DNA binding domain are from different transcription factors.

The first and second switch domains coupled to the transcription factor domains are introduced, e.g., to a cell, with a transgene that is operably linked to a promoter, e.g., a cell-specific, tissue-specific, or constitutive promoter, and transcriptional regulatory elements including one or more binding sites for the DNA binding domain coupled to the second switch domain. In some aspects, the first and second switch domains coupled to the transcription factor domains are introduced, e.g., to a cell, as polypeptides. In other aspects, the first and second switch domains coupled to the transcription factor domains are introduced, e.g., to a cell, by introducing nucleic acids, e.g., one or more vectors, encoding the first and second switch domains coupled to the transcription factor domains and causing said first and second switch domains coupled to the transcription factor domains to be expressed. For example, an FRB derived switch domain described herein, e.g., a mutant FRB, is coupled to the transactivation domain of the NFkB p65 transcription factor, e.g., amino acids 361-550. One or more, e.g., two or three, FKBP derived switch domains described herein, e.g., a mutant FKBP, are coupled to the DNA binding domain ZFHD1 of the Zif268 transcription factor, e.g., amino acids 333-390. In this aspect, the transgene to be expressed is operably linked to a promoter, e.g., an IL-2 promoter, and a plurality of ZFHD1 binding sites, e.g., 8-12, are upstream of the promoter. Upon administration of the dimerization molecule, the transactivation domain and DNA binding domain of the transcription factors coupled to the FKBP and FRB derived switch domains can associate. The ZFHD1 DNA binding domain(s) bind to the ZFHD1 binding sites located upstream of the desired transgene, and the transactivation domain is in sufficient proximity to initiate transcription of the transgene.

In some aspects, including in some aspects for treating a disease, e.g., cancer, the transgene can be a component of a gene editing system, e.g., a zinc finger nuclease gene editing system, a TALEN gene editing system, a CRISPR/Cas gene editing system or a meganuclease gene editing system, e.g., as described herein. In some aspects for treating a disease, e.g., a cancer, the transgene can be any therapeutic protein, e.g., an antibody, a growth hormone, a receptor or fragment thereof, a ligand, a cytokine, a secreted protein and the like, or a derivative or functional fragment thereof. In an aspect, nucleic acids encoding the dimerization switch-containing molecules and the transgene are introduced to the subject in need thereof using methods described herein. In another aspect, cells are engineered to express the dimerization switch-containing molecules and are capable of expressing the transgene, and the cells are delivered to the subject in need thereof using methods described herein.

In other aspects, the expression of any desired gene can be modulated using the dimerization switch coupled to the transcription factor domains as described above to ascertain the effects of expression of a gene on other pathways or biological processes, e.g., in cell culture or in an animal model.

(See, e.g., Fang, J., et al. (2007). “An antibody delivery system for regulated expression of therapeutic levels of monoclonal antibodies in vivo.” Mol Ther 15(6): 1153-1159; Indraccolo, S., et al. (2006). “Gene therapy of ovarian cancer with IFN-[alpha]-producing fibroblasts: comparison of constitutive and inducible vectors.” Gene Ther 13(12): 953-965; Nguyen, M., et al. (2007). “Rapamycin-regulated control of antiangiogenic tumor therapy following rAAV-mediated gene transfer.” Mol Ther 15(5): 912-920; Rivera, V. M., et al. (1996). “A humanized system for pharmacologic control of gene expression.” Nat Med 2(9): 1028-1032.)

Modulation of Signal Transduction

In one aspect, a dimerization switch described herein regulates signal transduction, e.g., cell proliferation, and can be used to regenerate or repair tissue. In an aspect, a first and second switch domain described herein is coupled, e.g., fused, to the intracellular portion of a receptor tyrosine kinase that mediates signalling, e.g., the intracellular signalling domain(s) through proliferation pathways, e.g., the P13K or AKT pathway. In such aspects, the expression of the dimerization switch containing molecules can be limited to a particular tissue or cell type that can benefit from cell proliferation, e.g., repair or regeneration, by introducing the nucleic acids encoding the dimerization switch-containing molecules operably linked to a cell-specific or tissue-specific promoter. In some aspects, the tissue that can be repaired or regenerated using the dimerization switch-containing molecules described herein is the liver. For example, a first and second switch domain described herein is coupled, e.g., fused, to Fgfr4 or FGFRIIIb, e.g., the intracellular kinase domain of Fgfr4 or FGFRIIIb. Upon administration of the dimerization molecule, the intracellular portions of the receptor associate, which causes activation of Akt and subsequent Akt-mediated signalling to stimulate cell proliferation.

Regulation of Protein Translocation

In one aspect, a dimerization switch described herein regulates protein translocation, e.g., to a membrane. These aspects provide a tool to investigate the function of particular protein of interest or the physiological consequences of modulating the function by sequestering the protein that is normally located or functioning intracellularly, or by activating a protein or pathways that are activated when the protein is localized at the membrane. The dimerization switch-containing molecules described here that regulate protein translocation can be used in cell culture studies, as well as introduced into in vivo models, e.g., mouse models, to investigate physiological consequences protein function at the membrane or the loss of intracellular protein function.

In an aspect, the first switch domain is coupled, e.g., fused, to a membrane anchoring domain, e.g., a molecule that is localized to the plasma membrane, e.g., a myristoyl group, or a myristoylation site, or a transmembrane domain. In such aspects, the transmembrane domain is derived from a naturally-occurring transmembrane protein and comprises the sequences that span or are sufficient for translocation to the plasma membrane. In other aspects, the transmembrane domain is a sequence of amino acids, e.g., 2 to 10 amino acids, comprising hydrophobic amino acids. In an aspect, the second switch domain is coupled to a protein of interest, or a functional fragment thereof, that is normally localized intracellularly, secreted, or otherwise not normally localized to the membrane. In such aspects, upon addition of the dimerization molecule, the protein of interest is localized to the membrane and therefore the normal intracellular function of the protein is inhibited, e.g., recapitulating loss of function. In other aspects, the protein of interest is localized to the membrane only in specific circumstances, e.g., during a specific signalling event or biological process. In such aspects, the protein of interest can be modified such that it lacks a membrane-interacting or transmembrane domain, e.g., a deletion mutant. In such aspects, upon addition of the dimerization molecule, the protein of interest is recruited to the plasma membrane, thereby recapitulating a particular signalling event or biological process.

For example, the first switch domain is coupled, e.g., fused, to a myristoylation group and the second switch domain is coupled to a mutant Akt that lacks the membrane-associating PH domain (e.g., aas 1-106). Dimerization of the switch domains results in Akt localization to the membrane to initiate Akt signalling. In another example, the first switch domain is coupled, e.g., fused, to a myristoylation group, and the second switch domain is coupled, e.g., fused, to the intracellular signaling region of Fgfr1. Dimerization of the switch domains results in dimerization or oligomerization of the Fgfr1 kinase domains, which initiates Fgfr1 signaling pathways. In these aspects, administration of the dimerization molecule allows for the investigation of specific signalling processes in particular cells. (See, e.g., Li, B. et al. (2002) “A novel conditional Akt ‘survival switch’ reversibly protects cells from apoptosis.” Gene Ther. 9(4):233-44; and Li, B., et al. (2007). “Conditional Akt activation promotes androgen-independent progression of prostate cancer.” Carcinogenesis 28(3): 572-583; and Welm, B. E., et al. (2002). “Inducible dimerization of FGFR1: development of a mouse model to analyze progressive transformation of the mammary gland.” J Cell Biol 157(4): 703-714.)

Regulation of Cre Recombination

In one aspect, the dimerization switch described herein can be used to regulate Cre recombination in generating transgenic animals, e.g., mice, comprising the Cre/loxP system. The Cre/LoxP system is known in the art and is used to generate mice with conditional expression of genes of interest. Current methods for regulating Cre recombination include using cell-specific promoters or inducible promoters, e.g., Tet-regulatable system, to regulate expression of the Cre recombinase.

In one aspect, a first switch domain described herein is coupled, e.g., fused, to a first fragment of the Cre recombinase, e.g., an N-terminal fragment of Cre, wherein the first fragment does not have substantial Cre activity. In an aspect, a second switch domain described herein is coupled, e.g., fused, to a second fragment of the Cre recombinase, e.g., a C-terminal fragment of Cre, wherein the second fragment does not have substantial Cre activity. The first and second fragments are selected such that, when associated together by dimerization of the switch domains in the presence of the dimerization molecule, results in Cre recombinase activity. For example, a first switch domain is coupled to a N-terminal fragment of Cre comprising amino acids 19-59, and a second switch domain is coupled to a C-terminal fragment of Cre comprising amino acids 60-343. (See, e.g., Jullien, N., et al. (2007). “Conditional transgenesis using Dimerizable Cre (DiCre)” PLoS One 2(12):e1355.)

Regulation of Protein Function

In one aspect, the dimerization switch described herein can be used to regulate protein function, e.g., by altering the stability or degradation of a protein of interest. In an aspect, a first switch domain is coupled to, e.g., fused to, a protein of interest that is degraded in the cell. In some aspects, a FRB derived switch domain is coupled to, e.g., fused to, a protein of interest, whereby the fusion with the FRB switch domain causes degradation of the protein. In an aspect, the second switch domain, e.g., FKBP, is not coupled to a second moiety, but upon addition of the dimerization molecule and association between the first and second switch domain inhibits or reduces the degradation of the protein of interest. (See, e.g., Stankunas, K., et al. (2003). “Conditional protein alleles using knockin mice and a chemical inducer of dimerization.” Mol Cell 12(6): 1615-1624).

Regulation of Post-Translational Modification

In one aspect, the dimerization switch described herein regulates post-translational modification. In an aspect, a first switch domain described herein is coupled, e.g., fused, to an enzyme that modifies proteins post-translationally. Examples of enzymes that modify proteins post-translationally include: sumoyltransferases, kinases, phosphatases, ubiquitin-transferring enzymes, neddylation enzymes, and glycosylases. In an aspect, the second switch domain described herein is coupled, e.g., fused, to a substrate of the enzyme that modifies proteins post-translationally. Upon addition of the dimerization molecule, the enzyme that mediates the post-translational modification is brought in sufficient proximity to modify the substrate. For example, a first switch domain is coupled to a sumoyltransferase, e.g., U9 symoyltransferase, and a second switch domain is coupled to a U9 sumoyltransferase substrate, e.g., STAT1, P53, CRSP9, FOS, CSNK2B. Dimerization by addition of a dimerization molecule induces sumoylation of the substrates. (See, e.g., Zimnik, S., et al. (2009). “Mutually exclusive STAT1 modifications identified by Ubc9/substrate dimerization-dependent SUMOylation.” Nucleic Acids Res 37(4): e30.).

Nuclear Localization

In some aspects, a FKBP:FRB dimerization switch or a dimerization switch described herein regulates protein translocation to and from the nucleus, e.g., of a cell, e.g., of a eukaryotic cell, e.g., of a mammalian cell. Without intending to be bound by theory, it is believed that in eukaryotic cells, macromolecules, e.g., proteins, move between the nucleus and cytoplasm through a large protein complex spanning the nuclear envelope referred to as the nuclear pore complex (NPC). The transport of proteins to and from the nucleus is often mediated by a family of transport receptors known as karyopherins. Karyopherins bind to their cargoes via recognition of nuclear localization signal (NLS) for nuclear import or nuclear export signal (NES) for export to form a transport complex that is passed though the NPC. These aspects provide, for example, a mechanism to direct a macromolecule, e.g., a protein, of interest into or out of the nucleus. The dimerization switch-containing molecules described here that regulate protein translocation to and from the nucleus can be used in, for example, cell culture studies, as well as introduced into in vivo models, e.g., mouse models, to investigate the role of localization of various protein in the nucleus or cytoplasm. The dimerization switch-containing molecules described here that regulate protein translocation to and from the nucleus can also be used to regulate the function of systems requiring the nuclear localization of one or more components of the system, e.g., of a gene editing system, e.g., as described herein.

In some aspects, the first or second switch domain is coupled, e.g., fused, to a nuclear localization sequence (NLS) comprising or derived from, e.g., a monopartite classical NLS, e.g., the SV40 large T antigen NLS (PKKKRRV; SEQ ID NO: 36) or, e.g., a bipartite classical NLS, e.g., the nucleoplasmin NLS (KRPAATKKAGQAKKKK; SEQ ID NO: 37). In another aspect, the NLS is derived from a naturally-occurring protein and comprises the amino acids that constitute the NLS or are sufficient to localize the protein to the nucleus. Such other suitable NLS sequences are known in the art (e.g., Sorokin, Biochemistry (Moscow) (2007) 72:13, 1439-1457; Lange J Biol Chem. (2007) 282:8, 5101-5). In some aspects, the other switch domain is coupled to a protein of interest, or a functional fragment thereof, that is normally localized in the cytoplasm, or otherwise not normally localized to the nucleus. In such aspects, upon addition of the dimerization molecule, the protein of interest is localized to the nucleus. In one aspect, the protein of interest is a protein that acts upon DNA, e.g., a gene or chromosome, e.g., is a transcription factor or protein with nuclease activity. In such aspects, upon addition of the dimerization molecule, the protein of interest is recruited to the nucleus where it can act upon DNA, e.g., regulate transcription or cleave a DNA substrate.

In some aspects the first or second switch domain is coupled, e.g., fused, to a NES, e.g., an NES known in the art.

In an aspect, the first switch domain is coupled, e.g., fused, to a NLS sequence, e.g., as described herein, and the second switch domain is coupled, e.g., fused, to a component of a gene editing system, e.g., as described herein. In an aspect the component of the gene editing system is a gene editing protein, e.g., as described herein, e.g., gene editing protein, e.g., a zinc finger nuclease, e.g., as described herein; e.g., a transcription activator-like effector nuclease (TALEN), e.g., as described herein; e.g., a CRISPR-associated nuclease, e.g., as described herein, e.g., Cas9, e.g., a Cas9 from S. pyogenes, e.g., as described herein; e.g., a meganuclease, e.g., as described herein. Without intending to be bound by theory, it is believed that NLS, e.g., one or more NLS, are important to localize the gene editing protein into the nucleus so that it can interact, e.g., cleave, the target nucleic acid. In such aspects, upon addition of the dimerization molecule, the gene editing protein is localized to the nucleus.

Regulation of Gene Editing Systems

The present invention provides a regulatable gene editing system comprising a gene editing dimerization switch. As used herein, the term “gene editing system” refers to a system comprising one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In known gene editing systems, for example, the one or more DNA-binding domains or components are associated with the one or more DNA-modifying domains or components, such that the one or more DNA-binding domains target the one or more DNA-modifying domains or components to a specific nucleic acid site. Polypeptide components of a gene editing systems are referred to herein as “gene editing proteins.”

Gene editing systems are known in the art, and include but are not limited to, zinc finger nucleases, transcription activator-like effector nucleases (TALENs); clustered regularly interspaced short palindromic repeats (CRISPR)/Cas systems, and meganuclease systems. Without wishing to be bound by theory, it is believed that the known gene editing systems may exhibit unwanted DNA-modifying activity which is detrimental to their utility in therapeutic applications. These concerns are particularly apparent in the use of gene editing systems for in vivo modification of genes or gene expression, e.g., where cells are engineered to constitutively express components of a gene editing system, such as through lentiviral or adenoviral vector transfection.

The present invention provides gene editing systems where the gene editing activity, e.g., the gene-modifying or -modulating activity, of the gene editing system can be regulated (e.g., turned “on” or “off”) through the use of a gene editing dimerization molecule, which optimizes the safety and efficacy of the therapeutic uses of the gene editing system. One aspect comprises a first gene editing switch domain is coupled, e.g., fused, to a DNA-binding domain of a gene editing system and a second gene editing switch domain is coupled, e.g., fused, to a DNA-modifying domain of a gene editing system. In the presence of a suitable gene editing dimerization molecule, e.g, as described herein, the first and second gene editing switch domains associate (e.g., form a complex), thereby causing the association of the DNA-binding domain and the DNA-modifying domain of the gene editing system.

DNA-Binding Domain

A “DNA-binding domain” of the present invention is a molecule or domain of a molecule that binds DNA, e.g., binds a specific sequence of DNA, e.g., binds a specific sequence of DNA comprising 1-50, e.g., 1-40, e.g., 1-30, e.g., a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-base pairs. The base pairs bound by the DNA-binding domain may be contiguous or may comprise sequences separated by sequences not targeted by the DNA-binding domain. In some aspects, the DNA-binding domain comprises one or more zinc fingers, e.g., one or more engineered zinc fingers, e.g., as described herein. In some aspects, the DNA-binding domain comprises one or more transcription activator-like effector (TALE) domains, e.g., one or more engineered TALE domains, e.g., as described herein. In some aspects, the DNA-binding domain is derived from a nuclease that has been engineered such that it binds DNA but does not have DNA-modifying activity, e.g., dCas9. Tsai (2014), Nat. Biotech. 32:569-577. In some aspects, the DNA-binding domain is derived from the domain of a protein capable of binding DNA, e.g., a nuclease or e.g., a transcription factor, that is responsible for the DNA-binding activity. In such aspects the DNA-binding domain does not have an activity other than DNA-binding activity. In some aspects, the DNA-binding domain is a nucleic acid, e.g., an RNA or DNA that hybridizes with a target nucleic acid.

DNA-Modifying Domain

A “DNA-modifying domain” of the present invention is a molecule or domain of a molecule that is capable of causing a change to the covalent structure of a DNA molecule. In some aspects, the change to the covalent structure of a DNA molecule is a cleavage i.e., a breakage, of the covalent backbone of a DNA molecule.

In some aspects, the DNA-modifying domain comprises a nuclease or catalytically active fragment thereof that is capable of introducing a double-strand break in DNA. In some aspects, the nuclease or catalytically active fragment is derived from a GIY-YIG homing endonuclease, e.g., is derived from I-Teel or, e.g., is derived from I-Bmol. Kleinstiver, B. P., Proc. Nat'l Acad. Sci. USA (2012) 109: 8061-8066; Edgell (2001) Proc Nat'l Acad Sci USA 98:7898-7903.

In some aspects, the DNA-modifying domain comprises a nuclease half-domain that, in conjunction with a second DNA-modifying domain (either identical or different) forms a complex capable of introducing a double-strand break in DNA. Nuclease half-domains may form homodimer or heterodimer complexes. In some aspects, the nuclease half-domain is derived from a Type IIS restriction enzyme. In some aspects, the nuclease half-domain is derived from FokI, e.g., a wt FokI half-domain (Wah et al., (1998) Proc Natl Acad Sci USA 95:10564-10569) or e.g., an engineered FokI half-domain, e.g., as described herein (e.g., as described in WO2007/139898; WO2011/097036; Doyon (2011) Nat. Methods, 8:74-79). In some aspects, the nuclease half-domain is derived from a PvuII restriction enzyme (Fonfara I (2012) Nucleic Acids Res 40:847-860; Schierling B, (2012) Nucleic Acids Res 40:2623-2638.

CRISPR/Cas Gene Editing System

“CRISPR” or “CRISPR/Cas” as used herein refers to a set of clustered regularly interspaced short palindromic repeats, or a system comprising such a set of repeats. “Cas”, as used herein, refers to a CRISPR-associated protein. A “CRISPR/Cas system” refers to a system derived from CRISPR and Cas which can be used to silence or modify a target gene.

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or modifying specific genes) in eukaryotes such as mice or primates. Wiedenheft et al. (2012) Nature 482: 331-8. This is accomplished by, for example, introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in gene editing applications in eukaryotic cells, the spacers are derived from the eukaryotic target gene sequence.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836. In some aspects, the Cas9 is derived from a S. pyogenes Cas9.

The CRISPR/Cas systems can thus be used to edit a target gene (adding, replacing or deleting one or more base pairs), or introducing a premature stop which thus decreases expression of a target gene. The CRISPR/Cas system can alternatively be used like RNA interference, turning off a target gene in a reversible fashion. In a mammalian cell, for example, the RNA can guide the Cas protein to a target promoter, sterically blocking RNA polymerases.

Artificial CRISPR/Cas systems that can be modified to comprise a gene editing switch as described herein are known in the art, e.g., are described in U.S. Publication No. 20140068797, and Cong (2013) Science 339: 819-823; are described in Tsai (2014) Nature Biotechnol., 32:6 569-576, U.S. Pat. Nos. 8,871,445; 8,865,406; 8,795,965; 8,771,945; and 8,697,359.

TALEN Gene Editing System

“TALEN” refers to a transcription activator-like effector nuclease, an artificial nuclease which can be used to edit a target gene.

TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence. Zhang (2011), Nature Biotech. 29: 149-153. By combining an engineered TALE with a DNA cleavage domain, a restriction enzyme can be produced which is specific to any desired DNA sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. Zhang (2011), Nature Biotech. 29: 149-153

To produce a TALEN, a TALE protein is fused to a nuclease (N), e.g., a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

TALEN can be used inside a cell to produce a double-stranded break (DSB) in a target nucleic acid, e.g., a site within a gene. A mutation can be introduced at the break site if the repair mechanisms improperly repair the break via non-homologous end joining. Huertas, P., Nat. Struct. Mol. Biol. (2010) 17: 11-16. For example, improper repair may introduce a frame shift mutation. Alternatively, foreign DNA can be introduced into the cell along with the TALEN; depending on the sequences of the foreign DNA and chromosomal sequence, this process can be used to modify a target gene, e.g., correct a defect in the target gene, thus causing expression of a repaired target gene, or e.g., introduce such a defect into a wt gene, thus decreasing expression of a target gene. Miller, J. C., (2011) Nat. Biotechnol. 29, 143-148 and Hockemeyer, D. (2011) Nat. Biotechnol. 29, 731-734.

TALEN gene editing systems that can be modified to comprise a gene editing switch as described herein are known in the art, e.g., as described in Zhang et al. (2011) Nature Biotech. 29: 149-53; Geibler et al. (2011) PLoS ONE 6: e19509.

Zinc Finger Nuclease Gene Editing System

“ZFN” or “Zinc Finger Nuclease” refer to a zinc finger nuclease, an artificial nuclease which can be used to edit a target gene.

Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. Sera (2002), Biochemistry, 41:7074-7081; Liu (2008) Bioinformatics, 24:1850-1857.

A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, e.g., via non-homologous end joining, leading to a decrease in the expression of a target gene in a cell. Alternatively, foreign DNA can be introduced into the cell along with the ZFN; depending on the sequences of the foreign DNA and chromosomal sequence, this process can be used to modify a target gene, e.g., correct a defect in the target gene, thus causing expression of a repaired target gene, or e.g., introduce such a defect into a wt gene, thus decreasing expression of a target gene, e.g., as described in WO2013/169802.

ZFN gene editing systems that can be modified to comprise a gene editing dimerization switch as described herein are known in the art and are described in, e.g. WO2008/0212107; Urnov (2005) Nature 435:646-651.

Meganuclease Gene Editing System

“Meganuclease” refers to a meganuclease, an artificial nuclease which can be used to edit a target gene.

Meganucleases are derived from a group of nucleases which recognize 15-40 base-pair cleavage sites. Meganucleases are grouped into families based on their structural motifs which affect nuclease activity and/or DNA recognition. Members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif form homodimers, whereas members with two copies of the LAGLIDADG motif are found as monomers. The GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). The NHN family, the members are defined by motifs containing two pairs of conserved histidines surrounded by asparagine residues (see Chevalier et al. (2001), Nucleic Acids Res. 29(18): 3757-3774).

Strategies for engineering a meganuclease with altered DNA-binding specificity, e.g., to bind to a predetermined nucleic acid sequence are known in the art. E.g., Chevalier et al. (2002), Mol. Cell., 10:895-905; Epinat et al. (2003) Nucleic Acids Res 31: 2952-62; Silva et al. (2006) J Mol Biol 361: 744-54; Seligman et al. (2002) Nucleic Acids Res 30: 3870-9; Sussman et al. (2004) J Mol Biol 342: 31-41; Rosen et al. (2006) Nucleic Acids Res; Doyon et al. (2006) J. Am Chem Soc 128: 2477-84; Chen et al. (2009) Protein Eng Des Sel 22: 249-56; Arnould S (2006) J Mol Biol. 355: 443-58; Smith (2006) Nucleic Acids Res. 363(2): 283-94.

A meganuclease can create a double-stranded break in the DNA, which can create a frame-shift mutation if improperly repaired, e.g., via non-homologous end joining, leading to a decrease in the expression of a target gene in a cell. Alternatively, foreign DNA can be introduced into the cell along with the Meganuclease; depending on the sequences of the foreign DNA and chromosomal sequence, this process can be used to modify a target gene, e.g., correct a defect in the target gene, thus causing expression of a repaired target gene, or e.g., introduce such a defect into a wt gene, thus decreasing expression of a target gene, e.g., as described in Silva et al. (2011) Current Gene Therapy 11:11-27.

Gene Editing Switch Domains

Gene Editing Dimerization Switches

According to the present invention, gene editing dimerization switches comprise a polypeptide comprising a first gene editing switch domain and a polypeptide comprising a second gene editing switch domain. A Gene editing dimerization switch can be non-covalent or covalent, depending on the form of interaction between the gene editing switch domains. Examples of gene editing dimerization switches (and their associated gene editing switch domains) are described herein.

### Non-Covalent Gene Editing Dimerization Switches

In a non-covalent gene editing dimerization switch, the gene editing dimerization molecule promotes a non-covalent interaction between the gene editing switch domains. Examples of non-covalent gene editing dimerization switches include the FKBP/FRAP-Based Dimerization Switches, GyrB-GyrB Based Dimerization Switches and Gibberelin-Based Dimerization Switches, described herein.

### FKBP/FRB-Based Gene Editing Dimerization Switches

In some aspects, the gene editing dimerization switch of the present invention is a FKBP/FRB based switch, e.g., as described herein. In some aspects, the gene editing dimerization switch comprises a dimerization switch. It is contemplated that any of the dimerization switches described herein are suitable for use as a gene editing dimerization switch. In some aspects, the FKBP/FRB based gene editing dimerization switch comprises a switch domain, e.g., as described herein in the sections entitled FRB MUTANTS and/or FKBP MUTANTS. In some aspects the first or second gene editing switch domain is a FRB mutant, e.g., as described herein, and the other gene editing switch domain is a FKBP mutant, e.g., as described herein. In other aspects, the FKBP/FBP-based gene editing dimerization switch comprises an FRB capable of forming a complex with a FKBP and AP21967.

In some aspects, the gene editing dimerization switch comprises one or more of the gene editing switch domains 1) to 10), below:
1) In an aspect, the first gene editing switch domain comprises one or more mutations each of which enhances formation of a complex between a first gene editing switch domain, a second gene editing switch domain (e.g., a FKBP derived switch domain), and a gene editing dimerization molecule (e.g., a rapamycin, or a rapalog, e.g., RAD001). In an aspect, the enhancement is additive or more than additive.
2) In an aspect, the first gene editing switch domain comprises a mutation at E2032, e.g., E2032I or E2032L, and at T2098, e.g., T2098L.
3) In an aspect, the gene editing first switch domain comprises the mutation E2032I, and further comprises a mutation at one or a plurality of L2031, S2035, R2036, F2039, G2040, T2098, W2101, D2102, Y2105, or F2108.
4) In an aspect, the first gene editing switch domain comprises a mutation at E2032I and at T2098. In one aspect the mutation at T2098 is T2098L.
5) In an aspect, the first gene editing switch domain comprises the mutation at E2032L, and further comprises a mutation at one or more of L2031, S2035, R2036, F2039, G2040, T2098, W2101, D2102, Y2105, or F2108.
6) In an aspect, the first gene editing switch domain comprises a mutation at E2032L and at T2098. In one aspect the mutation at T2098 is T2098L.
7) In an aspect, the first gene editing switch domain comprises a T2098 mutation and one or more mutations at L2031, E2032, R2036, G2040, or F2108. In one aspect the mutation at T2098 is T2098L.
8) In an aspect the gene editing first switch domain comprises a mutation at T2098L and at E2032. In an aspect the mutation at E2032 is E2032I. In another aspect the mutation at E2032 is E2032L.
9) In an aspect the second gene editing switch domain comprises one or more mutations that enhance the formation of a complex between the first gene editing switch domain, the second gene editing switch domain, and the gene editing dimerization molecule, rapamycin, or a rapalog, e.g., RAD001. In an aspect the second gene editing switch domain comprises one or more mutations at Q53, I56, W59, Y82, G89, I90, I91, K44, P45, or H87. In an aspect, the second gene editing switch domain comprises one or more mutations at Q53, I56, W59, Y82, H87, G89, or I90.
10) the first gene editing switch domain comprises one or more mutations that enhance the formation of a complex between the first gene editing switch domain, the second gene editing switch domain, and the gene editing dimerization molecule, rapamycin, or a rapalog, e.g., RAD001; and (B) the second gene editing switch domain comprises one or more mutations that enhance the formation of a complex between the first gene editing switch domain, the second gene editing switch domain, and the gene editing dimerization molecule, rapamycin, or a rapalog, e.g., RAD001.

In some aspects, the first gene editing switch domain comprises a first switch domain as described herein, or a polypeptide comprising an FRB fragment or analog thereof as described herein in the section titled FRB MUTANTS.

In an aspect, the second gene editing switch domain comprises a second switch domain as described herein, or a polypeptide comprising a FKBP fragment or analog thereof as described herein in the section titled FKBP MUTANTS.

In an aspect, the first gene editing switch domain comprises a first switch domain as described herein, or a polypeptide comprising an FRB fragment or analog thereof as described herein and the second gene editing switch domain comprises a second switch domain as described herein, or a polypeptide comprising a FKBP fragment or analog thereof as described herein.

In some aspects the gene editing dimerization switch comprises a first gene editing switch domain that differs at no more than 1, 2, 3, 4, 5, 6, 7, 8, 9, or 10 amino acid residues from the sequence of SEQ ID NO:2.

In some aspects, the gene editing dimerization switch comprises a first gene editing switch domain comprising 30, 35, 40, 45, 50, 55, 60, 70, 75, 80, 85 or 90 amino acids of the sequence of FRB, SEQ ID NO:2.

In some aspects, the gene editing dimerization switch comprises a second gene editing switch domain that differs at no more than 1, 2, 3, 4, 5, 6, 7, 8, 9, or 10 amino acid residues from the sequence of SEQ ID NO:1 or 3.

In some aspects, the gene editing dimerization switch comprises a second gene editing switch domain comprising 30, 35, 40, 45, 50, 55, 60, 70, 75, 80, 85 or 90 amino acids of the sequence of FKBP, SEQ ID NO:1 or 3.

In some aspects, the gene editing dimerization switch comprises a first gene editing switch domain comprising T2098L and E2032I. In some aspects, the gene editing dimerization switch comprises a first gene editing switch domain or T2098L and E2032L.

In some aspects, the gene editing dimerization switch comprises a first gene editing switch domain comprising T2098L and E2032I, or T2098L and E2032L, and the second gene editing switch domain comprises one or more mutations at Y26, F36, D37, R42, K44, P45, F46, Q53, E54, V55, I56, W59, Y82, H87, G89, I90, I91, and F99, e.g., one or more mutations at Y26, F36, D37, R42, F46, Q53, E54, V55, I56, W59, Y82, H87, G89, I90, or F99.

### AP21967 and FKBP/FRB Gene Editing Dimerization Switches

In one aspect, the gene editing dimerization molecule is a rapamycin analog, e.g., AP21967, that does not mediate formation of a complex comprising wild-type endogenous FRAP, e.g., FRB, but that does mediate formation of a complex comprising a modified FRB (e.g., a FRB comprising one or more mutations). While not wishing to be bound by theory, it is believed that a gene editing dimerization molecule lacking the ability to mediate formation of a complex comprising endogenous FRB reduces its immunosuppressive activity. An exemplary modified FRB contains a single amino acid change (T2098L) to SEQ ID NO: 2. Incorporation of this mutation into the FRB component of a gene editing dimerization switch allows AP21967 to be used as a gene editing dimerization molecule.

In an aspect, one gene editing switch domain comprises sequence from FKBP having the ability to form a complex with a FRB and AP21967, and/or having the ability to form a complex with a FRB gene editing switch domain and a rapamycin analog, e.g., AP21967, wherein the FRB or FRB gene editing switch domain comprises sequence from an FRB that is capable of forming a complex with the FKBP gene editing switch domain and AP21967.

In an aspect, one gene editing switch domain comprises amino acid residues disclosed in SEQ ID NO: 1 and one gene editing switch domain comprises amino acid residues disclosed in SEQ ID NO: 2.

In some aspects the gene editing switch domain having the ability to form a complex with a second gene editing switch domain and a rapamycin analog, e.g., AP21967 will have at least 70, 75, 80, 85, 90, 95, 96, 97, 98, or 99% identity with the FKBP sequence of SEQ ID NO: 1. In some aspects, the gene editing switch domain having the ability to form a complex with a second gene editing switch domain and a rapamycin analog, e.g., AP21967, will differ by no more than 30, 25, 20, 15, 10, 5, 4, 3, 2, or 1 amino acid residues from the corresponding the sequence of SEQ ID NO: 1

In some aspects the gene editing switch domain having the ability to form a complex between a gene editing switch domain and a rapamycin analog, e.g., AP21967 will have at least 70, 75, 80, 85, 90, 95, 96, 97, 98, or 99% identity with the FRB sequence of SEQ ID NO: 17. In some aspects, the gene editing switch domain having the ability to form a complex between a gene editing switch domain and a rapamycin analog, e.g., AP21967, will differ by no more than 30, 25, 20, 15, 10, 5, 4, 3, 2, or 1 amino acid residues from the corresponding FRB sequence of SEQ ID NO: 17.

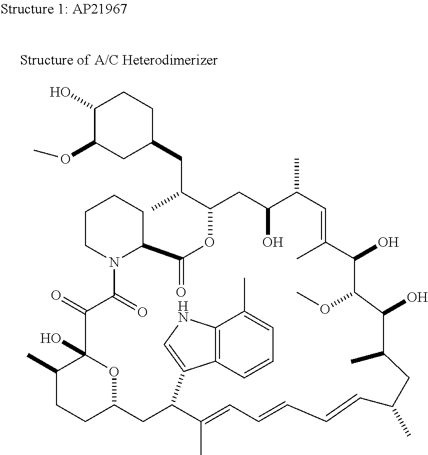

Similar switches have been used to control the localization and activity of signaling domains as described above (see, e.g., Graef, I. A., Holsinger, L. J., Diver, S., Schreiber, S. L. & Crabtree, G. R. (1997) Proximity and orientation underlie signaling by the non-receptor tyrosine kinase ZAP70. Embo J 16: 5618-28).

The present invention also provides methods for screening for other candidate sequences for use as a gene editing switch domain having the ability to form a complex between a gene editing switch domain and a rapamycin analog, e.g., AP21967. Such candidate sequences can be evaluated by screening for AP21967-mediated complex formation, e.g., in an assay similar to those described in Examples 1 and 2.

GyrB-GyrB Based Gene Editing Dimerization Switches

In some aspects, the gene editing dimerization switch of the present invention is a GyrB-GyrB based gene editing dimerization switch, e.g., as described herein. Coumermycin, a product of Streptomyces, binds the amino-terminal 24K subdomain of the B subunit of bacterial DNA gyrase, GyrB. Coumermycin binds two GyrB subunits, see, e.g., Rarrar et al., (1996) Activation of the Raf-1 kinase cascade by coumermycin induced dimerization, Nature 383: 178; Gilbert et al. (1994) The 24 kDa N-terminal sub-domain of the DNA gyrase B protein binds coumarin drugs, Molecular Microbiology 12: 365. Thus, coumermcyn can be used as a gene editing dimerization molecule in a homodimerization gene editing dimerization switch comprising gene editing switch domains that comprise a coumermycin binding sequence of GyrB.

In an aspect, the gene editing switch domain comprises a coumermycin binding sequence from the 24 K Da amino terminal sub-domain of GyrB.

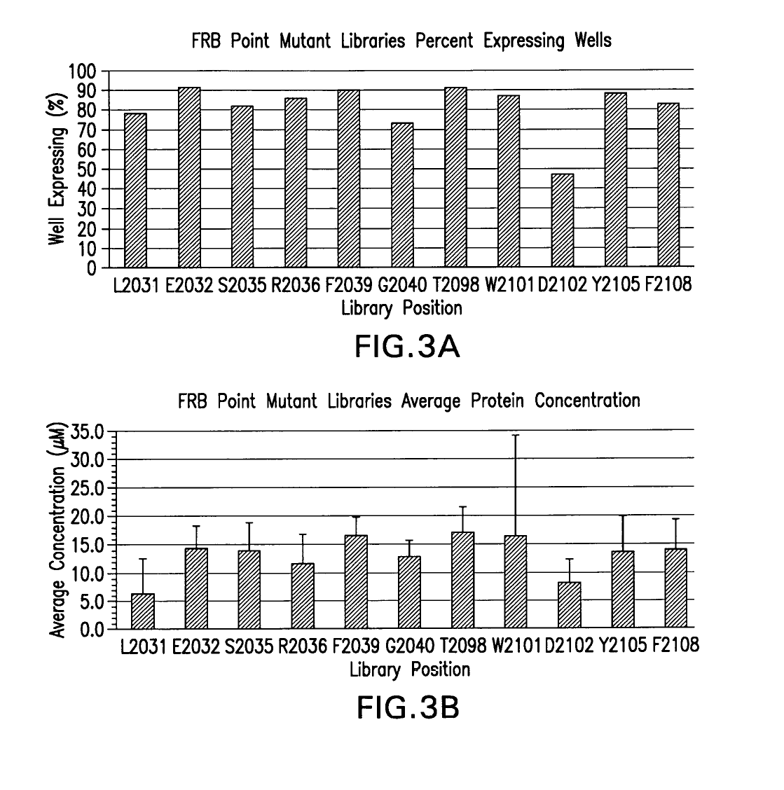

In some aspects, the gene editing switch domain, or a coumermycin binding sequence of the gene editing switch domain thereof, will have at least 70, 75, 80, 85, 90, 95, 96, 97, 98, or 99% identity with the GyrB sequence of Rarrar et al., (1996). In some aspects, the gene editing switch domain, or a coumermycin binding sequence thereof, will differ by no more than 30, 25, 20, 15, 10, 5, 4, 3, 2, or 1 amino acid residues from the corresponding sequence of Rarrar et al., (1996). See, e.g., FIG. 3.

The present invention also provides methods for screening for other candidate sequences for use as a GyrB-GyrB based gene editing switch domain. For example, candidate sequences can be evaluated by incorporation into a system such as that described in Rarrar et al., (1996).

In such aspects, a suitable gene editing dimerization molecule is a coumermycin, e.g., Structure 2.

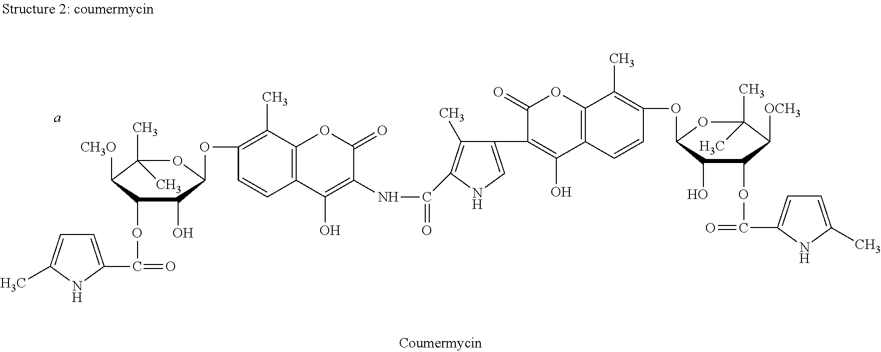

Gibberellin-Based Gene Editing Dimerization Switches

In some aspects, the gene editing dimerization switch of the present invention is a gibberellin based gene editing dimerization switch, e.g., as described herein. Gibberellins are plant hormones that regulate plant growth and development. Gibberellin binds to its receptor, gibberellin insensitive dwarf1 (GID1) and induces a conformational change in GID1. The new conformation allows GID1 to bind another protein, gibberellin insentivive (GAI). Gibberellin, or a giberellin analog, e.g., GA₃, or AM/GA₃, can be used to dimerize a gene editing switch domain comprising GA₃ binding sequence from GID1 (a GIDI gene editing switch domain) and a gene editing switch domain comprising sequence from GAI sufficient to bind GA₃-bound GID1. GA₃-AM can cross the plasma membrane of target cells. Once inside the cells, GA₃-AM is cleaved by an esterase to form GA₃. See Miyamoto et al. (2010) Rapid and orthogonal logic gating with a gibberellins-induced dimerization system, Nat. Chem. Biol. 8:465.

In an aspect, one gene editing switch domain (a GAI gene editing switch domain) comprises a sequence of GAI sufficient to bind to a gibberellin analog, e.g., GA₃, and once bound to the analog, e.g., GA₃, bind to GID1; and one gene editing switch domain (a GID gene editing switch domain) comprises sequence of GID1 sufficient to bind to a GAI gene editing switch domain bound to a gibberellin analog, e.g., GA₃.

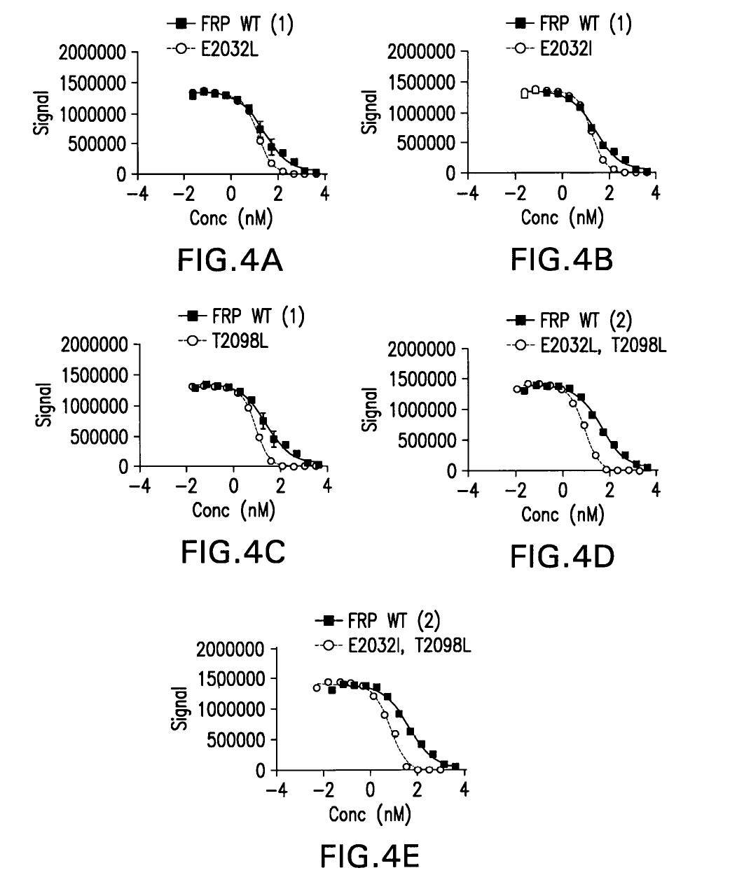

In some aspects, a GAI gene editing switch domain, or a sequence of GAI is sufficient to bind to a gibberellin analog, e.g., GA₃, and once bound to the analog, e.g., GA₃, bind to GID1, thereof, will have at least 70, 75, 80, 85, 90, 95, 96, 97, 98, or 99% identity with a GAI sequence of Miyamoto et al. (2010); or will differ by no more than 30, 25, 20, 15, 10, 5, 4, 3, 2, or 1 amino acid residues from the corresponding a sequence of Miyamoto et al. (2010). See, e.g., FIG. 4.

In some aspects, a GID1 gene editing switch domain, or a sequence of GID1 sufficient to bind to a GAI gene editing switch domain, thereof, will have at least 70, 75, 80, 85, 90, 95, 96, 97, 98, or 99% identity with the GID1 sequence of Miyamoto et al. (2010); or will differ by no more than 30, 25, 20, 15, 10, 5, 4, 3, 2, or 1 amino acid residues from the corresponding of Miyamoto et al. (2010).

The present invention also provides methods for screening for other candidate sequences for use as a GAI or GID1 gene editing switch domain. For example, candidate sequences can be evaluated by incorporating the candidate sequence into a system such as that described in Miyamoto et al. (2010).

In such aspects, a suitable gene editing dimerization molecule is gibberellin, or a giberellin analog, e.g., GA₃, or AM/GA₃

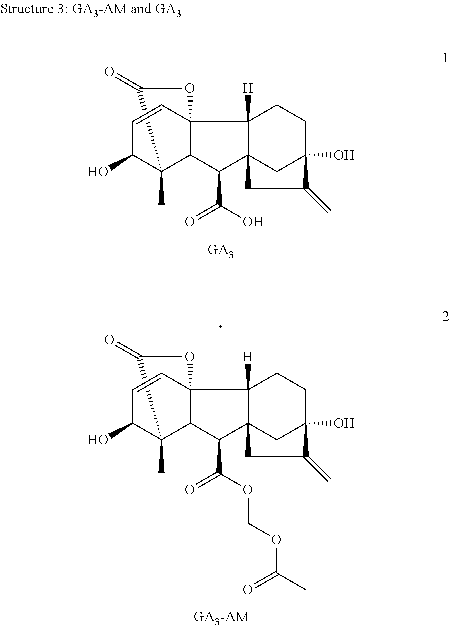 [SMILES: C=C1C[C@]23C[C@@]1(O)CC[C@H]2[C@@]12C=C[C@H](O)[C@@](C)(C(=O)O1)C2[C@@H]3C(=O)O ; C=C1C[C@]23C[C@@]1(O)CC[C@H]2[C@@]12C=C[C@H](O)[C@@](C)(C(=O)O1)C2[C@@H]3C(=O)OCOC(C)=O]

### Covalent Gene Editing Dimerization Switches

In a covalent gene editing dimerization switch, the gene editing dimerization molecule promotes a covalent interaction between the gene editing switch domains. In an aspect, a gene editing dimerization switch comprises first and second gene editing switch domains, which, upon contact with a gene editing dimerization molecule, are covalently coupled to one another. In some aspects, a covalent gene editing dimerization switch is a homodimerization switch, wherein the gene editing dimerization molecule covalently couples a first and second gene editing switch domain having the same structure. In some aspects of a covalent homodimerization switch, the linking molecule comprises a first and second reactive group, each of which can bind to and form a covalent bond with a gene editing switch domain, thereby covalently linking the gene editing switch domains. The first and second reactive groups can have the same structure or different structures. In some aspects, a covalent gene editing dimerization switch is a heterodimerization switch, wherein the gene editing dimerization molecule covalently couples first and second gene editing switch domains having structures that differ from one another. In some aspects of a covalent heterodimerization switch, the linking molecule can have a first reactive group that covalently binds the first gene editing switch domain, but not the second gene editing switch domain, and a second reactive group that covalently binds the second gene editing switch domain, but not the first gene editing switch domain. In some aspects, the gene editing dimerization molecule comprises an additional moiety that alters its solubility or cell permeability. E.g., in the case of an intracellular covalent heterodimerization switch, the dimerization molecule can comprise a moiety that optimizes the cell permeability of the dimerization molecule.

A Halotag/SNAP-tag switch is an example of a covalent heterodimerization switch. In an aspect, the gene editing dimerization molecule comprises a first reactive group, e.g., an O6-benzylguanine reactive group, that reacts covalently with a SNAP-tag domain, a second reactive group, e.g., a chloroalkane reactive group, that reacts with a Halotag domain, and a moiety that renders the gene editing dimerization molecule cell permeable.

Covalent dimerization switches are described in Erhart et al., 2013 Chem Biol 20(4): 549-557. HaXS species described therein are useful as gene editing dimerization molecules in a Halotag/SNAP-tag switch. In some aspects, a covalent dimerization molecule minimizes potential kinetic limitations related to off rates and need for accumulation of non-covalent gene editing dimerization molecules in the cell as prerequisites to activation of the required signal cascades, e.g., for T-cell mediated killing.

In an aspect, a Halotag/SNAP-tag gene editing dimerization switch comprises a first gene editing switch domain comprising a Halo-Tag, e.g., SEQ ID NO: 38, or a functional derivative or fragment thereof, and a second gene editing switch domain comprising a SNAP-Tag, e.g., SEQ ID NO: 39, or a functional derivative or fragment thereof. In some aspects the gene editing dimerization molecule comprises reactive groups for linking a Halo-Tag with a SNAP-Tag along with a cell penetrating core. Structure 5 depicts a gene editing dimerization molecule suitable for use in this system.
A Halo-tag Domain (SEQ ID NO: 38) (E.g., Genbank accession number ADN27525.1, residues 3 to 297)
eigtgfpfdphyvevlgermhyvdvgprdgtpvlflhgnptssyvwrniiphvapthrciapdligmgksdkpdlgyffddhvrf mdafiealgleevvlvihdwgsalgfhwakrnpervkgiafmefirpiptwdewpefaretfqafrttdvgrkliidqnvfiegtlpm gvvrpltevemdhyrepflnpvdreplwrfpnelpiagepanivalveeymdwlhqspvpkllfwgtpgvlippaeaarlakslpn ckavdigpglnllqednpdligseiarwlstleisg
A SNAP-tag domain (SEQ ID NO: 39) (E.g., Genbank accession number AIQ78245.1 residues 172 to 353)
Mdkdcemkrttldsplgklelsgceqglhriiflgkgtsaadavevpapaavlggpeplmqatawlnayfhqpeaieefpvpalhh pvfqqesftrqvlwkllkvvkfgevisyshlaalagnpaataavktalsgnpvpilipchrvvqgdldvggyegglavkewllaheg hrlgkpglg

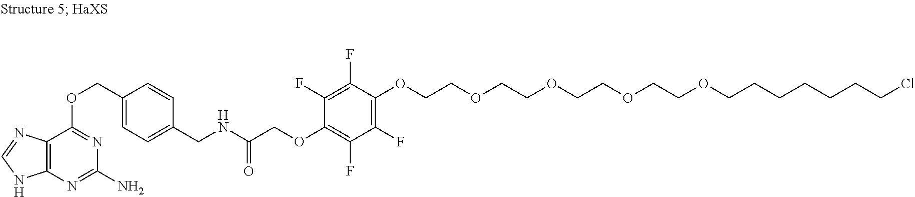 [SMILES: Nc1nc(OCc2ccc(CNC(=O)COc3c(F)c(F)c(OCCOCCOCCOCCOCCCCCCCCl)c(F)c3F)cc2)c2nc[nH]c2n1]

In an aspect, one gene editing switch domain comprises amino acid residues disclosed in SEQ ID NO: 38 and one gene editing switch domain comprises amino acid residues disclosed in SEQ ID NO: 39.

In some aspects the first gene editing switch domain, will have at least 70, 75, 80, 85, 90, 95, 96, 97, 98, or 99% identity with the sequence of SEQ ID NO: 38. In some aspects, the first gene editing switch domain, will differ by no more than 30, 25, 20, 15, 10, 5, 4, 3, 2, or 1 amino acid residues from the corresponding the sequence of SEQ ID NO: 38.

In some aspects the second gene editing switch domain, will have at least 70, 75, 80, 85, 90, 95, 96, 97, 98, or 99% identity with the sequence of SEQ ID NO: 39. In some aspects, the second gene editing switch domain, will differ by no more than 30, 25, 20, 15, 10, 5, 4, 3, 2, or 1 amino acid residues from the corresponding the sequence of SEQ ID NO: 39.

In such aspects, a suitable gene editing dimerization molecule is HaXS.

The present invention also provides methods for screening for other candidate sequences for use as a Halo-tag or SNAP-tag gene editing switch domain. For example, candidate sequences can be evaluated by incorporating the candidate sequence into a system such as that described herein.

Uses for Gene Editing Dimerization Switch-Containing Gene Editing Systems

In some aspects, a gene editing system, e.g., as described herein, comprising a gene editing dimerization switch can be used to create an allogeneic immune cell, e.g., a T-cell or NK cell, e.g., an allogeneic immunce cell lacking expression of a functional T cell receptor (TCR) and/or human leukocyte antigen (HLA), e.g., HLA class I and/or HLA class II.

In some aspects, a gene editing system, e.g., as described herein, comprising a gene editing dimerization switch can be used to create a T cell lacking a functional TCR, e.g., engineered (e.g., in the presence of a gene editing dimerization molecule) such that it does not express any functional TCR on its surface, such that it does not express one or more subunits, e.g., a TCRα and/or TCRβ that comprise a functional TCR, or such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

In some aspects, a gene editing system, e.g., as described herein, comprising a gene editing dimerization switch can be used to engineer (e.g., in the presence of a gene editing dimerization molecule) a T cell such that it does not express a functional HLA on its surface, or where cell surface expression of HLA, e.g., HLA class I and/or HLA class II, is downregulated. For example, a gene editing dimerization switch of the present invention comprises a first polypeptide comprising a DNA-binding domain that recognizes a nucleic acid sequence of an HLA gene (e.g., a zinc finger engineered to recognize a nucleic acid sequence of an HLA gene) coupled, e.g., fused, to a first gene editing switch domain, e.g., a FKBP-derived switch domain, and a second polypeptide comprising a DNA-modifying domain (e.g., a nuclease, e.g., a FokI half domain) coupled, e.g., fused, to a second gene editing switch domain, e.g., a FRB-derived switch domain. The function of such a gene editing system can be regulated by addition of an effective amount of a gene editing dimerization molecule, e.g., rapamycin or a rapalog, e.g., RAD001.

In some aspects, two or more gene editing systems, e.g., as described herein, each comprising a gene editing dimerization switch can be used to regulate expression of two or more genes. In some aspects, the same gene editing dimerization switch is used in each of the two or more gene editing systems. In some aspects, it may be desirable to provide only a single DNA-modifying domain coupled, e.g., fused to, a first or second gene editing switch domain, wherein said first or second gene editing switch domain has the ability to associate with two or more unique DNA-binding domains each fused to a gene editing switch domain. Without wishing to be bound by theory, it is believed that administration of a suitable gene editing dimerization molecule allows the DNA-modifying domain to associate with each of the DNA-binding domains, thereby directing the gene editing systems to each of their target genes.

In other aspects, different gene editing dimerization switches are used in each of the two or more gene editing systems, such that regulation of each gene editing system can be independently controlled.

In one aspect, two gene editing systems comprising one or more gene editing dimerization switches are used to regulate, e.g., inhibit, expression of both a functional TCR and a functional HLA, e.g., HLA class I and/or HLA class II.

In some aspects, a gene editing system, e.g., as described herein, comprising a gene editing dimerization switch can be used to regulate, e.g., downregulate, inhibit or repress expression of an inhibitory molecule. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4 and TGFR beta. Inhibition of an inhibitory molecule in a cell, e.g., with the use of a gene editing system comprising a gene editing dimerization switch as described herein, can improve the function of the cell.

In some aspects, a gene editing system, e.g., as described herein, comprising a gene editing dimerization switch (or nucleic acid encoding said gene editing dimerization switch, or cell comprising said gene editing dimerization switch, e.g., as described herein) is used to treat a disorder associated with abberant gene expression, e.g., a cancer or a genetic disorder. Examples of cancers that may be treated with the compositions of the present invention include breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis. Examples of genetic disorders are described on the website of the National Institutes of Health under the topic subsection Genetic Disorders (website at health.nih.gov/topic/GeneticDisorders). Other examples include ocular defects caused by genetic mutations, including those described in Genetic Diseases of the Eye, Second Edition, edited by Elias I. Traboulsi, Oxford University Press, 2012. Preferably the genetic disorder is selected from the group consisting of epidermolysis bullosa, recessive dystrophic epidermolysis bullosa (RDEB), osteogenesis imperfecta, dyskeratosis congenital, a mucopolysaccharidosis, muscular dystrophy, cystic fibrosis (CFTR), fanconi anemia, a sphingolipidosis, a lipofuscinosis, adrenoleukodystrophy, severe combined immunodeficiency, sickle-cell anemia and thalassemia.

In some aspects, a gene editing system, e.g., as described herein, comprising a gene editing dimerization switch (or nucleic acid encoding said gene editing dimerization switch, or cell comprising said gene editing dimerization switch, e.g., as described herein) is used to treat a lysosomal storage disorder. Examples of liposomal storage disorders include Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease, GM1 gangliosidosis, I-Cell disease/Mucolipidosis II, Infantile Free Sialic Acid Storage Disease/ISSD, Juvenile Hexosaminidase A Deficiency, Krabbe disease, Metachromatic Leukodystrophy, Mucopolysaccharidoses disorders, e.g., Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type AMPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV; Multiple sulfatase deficiency, Niemann-Pick Disease, Neuronal Ceroid Lipofuscinoses, CLN6 disease, Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, Pycnodysostosis, Sandhoff disease/Adult Onset/GM2 Gangliosidosis, Schindler disease, Salla disease/Sialic Acid Storage Disease, Tay-Sachs/GM2 gangliosidosis, and Wolman disease

### Nucleic Acids and Vectors

Nucleic acid sequences encoding a dimerization switch-containing molecule, e.g., polypeptide, described herein can be obtained using standard synthetic and/or recombinant techniques. Desired nucleic acid sequences may be isolated and sequenced from appropriate source cells or can be synthesized using nucleotide synthesizer or PCR techniques.

The expression of natural or synthetic nucleic acids encoding a dimerization switch-containing molecule described herein is typically achieved by operably linking a nucleic acid encoding the dimerization switch-containing molecule polypeptide or portions thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. In one aspect, the the vector comprising the nucleic acid encoding the dimerization switch-containing molecule of the invention is a DNA, a RNA, a plasmid, an adenoviral vector, a lentivirus vector, or a retrovirus vector.

Viral vector technology is well known in the art and is described, for example, in Sambrook et al., 2012, MOLECULAR CLONING: A LABORATORY MANUAL, volumes 1-4, Cold Spring Harbor Press, N.Y.), and in other virology and molecular biology manuals. Viruses, which are useful as vectors include, but are not limited to, retroviruses, adenoviruses, adeno-associated viruses, herpes viruses, and lentiviruses. Selection of an appropriate vector will depend mainly on the size of the nucleic acids to be inserted into the vector and the particular host cell to be transformed with the vector. Each vector contains various components, depending on its function (amplification or expression of heterologous nucleic acid sequence, or both) and its compatibility with the particular host cell in which it resides. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193). Other elements that may be included in the vector include a ribosomal binding site, a signal sequence, a transcriptional termination site, a tag, and a reporter gene.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to desired host cells, or cells of the subject, either in vivo or ex vivo. A number of retroviral systems are known in the art. In some aspects, adenovirus vectors are used. A number of adenovirus vectors are known in the art. In one aspect, adeno-associated virus (AAV) vector, e.g., an AAV2, AAV3, AAV4, AAV5, AAV6, AAV7, AAV8, or AAV9 vector, or any modified vectors thereof. In one aspect, lentivirus vectors are used.

### Expression in Cells

The present invention provides dimerization switches and gene editing dimerization switches useful in engineering cells to express a dimerization switch-containing molecule or a gene editing dimerization switch-containing molecule, and in applications involving the use of such engineered cells. The cells may be eurkaryote cells, e.g., insect, worm or mammalian cells. Suitable mammalian cells include, but are not limited to, equine, bovine, ovine, canine, feline, murine, non-human primate cells, and human cells.

Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Other cells for use in the present invention include stem and progenitor cells, such as hematopoietic, neural, stromal, muscle, hepatic, pulmonary, gastrointestinal and mesenchymal stem or progenitor cells. In some aspects using hematopoietic cells, the hematopoietic cells may include any of the nucleated cells which may be involved with the erythroid, lymphoid or myelomonocytic lineages, as well as myoblasts and fibroblasts, and immune effector cells, e.g., T cells and NK cells. The cells may be autologous cells, syngeneic cells, allogeneic cells and even in some cases, xenogeneic cells with respect to an intended host organism.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a nucleic acid into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, N.Y.). A preferred method for the introduction of a polynucleotide into a host cell is lipofection, e.g., using Lipofectamine (Life Technologies).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some aspects, host cells can be modified ex vivo with a nucleic acid, e.g., vector, comprising the dimerization switch-containing molecules described herein. Cells which have been modified ex vivo with the vector may be grown in culture under selective conditions and cells which are selected as having the desired construct(s) may then be expanded and further analyzed, using, for example, the polymerase chain reaction for determining the presence of the construct in the host cells and/or assays for the production of the desired gene product(s). Once modified host cells have been identified, they may then be used as planned, e.g. grown in culture or introduced into a host organism.

Depending upon the nature of the cells, the cells may be introduced into a host organism, e.g. a mammal, e.g., a human, in a wide variety of ways. Hematopoietic cells may be administered by injection into the vascular system, there being usually at least about 10⁴ cells and generally not more than about 10¹⁰ cells. The number of cells which are employed will depend upon a number of circumstances, the purpose for the introduction, the lifetime of the cells, the protocol to be used, for example, the number of administrations, the ability of the cells to multiply, the stability of the therapeutic agent, the physiologic need for the therapeutic agent, and the like. Generally, for myoblasts or fibroblasts for example, the number of cells will be at least about 10⁴ and not more than about 10⁹ and may be applied as a dispersion, generally being injected at or near the site of interest. The cells will usually be in a physiologically-acceptable medium. Cells engineered in accordance with this invention may also be encapsulated, e.g. using conventional biocompatible materials and methods, prior to implantation into the host organism or patient for the production of a therapeutic protein.

In other aspects, the cells can be engineered to express the dimerization switch-containing molecules in vivo. For this purpose, various techniques have been developed for modification of target tissue and cells in vivo. A number of viral vectors have been developed, such as adenovirus, adeno-associated virus, and retroviruses, as discussed above, which allow for transfection and, in some cases, integration of the virus into the host. See, for example, Dubensky et al. (1984) Proc. Natl. Acad. Sci. USA 81, 7529-7533; Kaneda et al., (1989) Science 243, 375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The vector may be administered by injection, e.g. intravascularly or intramuscularly, inhalation, or other parenteral mode. Non-viral delivery methods such as administration of the DNA via complexes with liposomes or by injection, catheter or biolistics may also be used.

In accordance with in vivo genetic modification, the manner of the modification will depend on the nature of the tissue, the efficiency of cellular modification required, the number of opportunities to modify the particular cells, the accessibility of the tissue to the nucleic acid, e.g., vector, composition to be introduced, and the like. Nucleic acid introduction need not result in integration. In some situations, transient maintenance of the introduced nucleic acids described herein may be sufficient. In this way, one could have a short term effect, where cells could be introduced into the host and then turned on after a predetermined time, for example, after the cells have been able to home to a particular site.

### Pharmaceutical Compositions and Treatments

Pharmaceutical compositions may comprise dimerization switch-containing molecules, e.g., a polypeptide or a nucleic acid encoding the dimerization switch-containing molecules, e.g., a vector encoding the dimerization switch-containing molecules, or a cell comprising the dimerization switch-containing molecules, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In an aspect, the pharmaceutical compositions are formulated for intravenous administration.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, disease state, e.g., tumor size, extent of infection or metastasis, and condition of the patient (subject). Compositions may also be administered multiple times at these dosages. The optimal dosage and treatment regime for a particular patient can be determined by one skilled in the art of medicine by monitoring the patient for signs of disease and adjusting the treatment accordingly.

The administration of the dimerization molecule may be carried out in any convenient manner, including by aerosol inhalation, injection, ingestion, transfusion, or implantation. In an aspect the dimerization molecule is administered orally. The dimerization molecule may be administered to a patient transarterially, subcutaneously, intradermally, intratumorally, intranodally, intramedullary, intramuscularly, by intravenous (i.v.) injection, or intraperitoneally. In an aspect, the dimerization molecule is administered orally, e.g., in tablet form. In an aspect, the dimerization molecule is administered by intradermal or subcutaneous injection. In an aspect, an aspect the dimerization molecule is administered by i.v. injection.

In an aspect, the dimerization molecule is administered after the composition comprising the dimerization switch, e.g., nucleic acids encoding the dimerization switch or cells comprising the dimerization switch, have been administered to the patient. In some aspects where the dimerization switch composition comprises cells comprising the dimerization switch-containing molecules, the dimerization switch composition is infused into the patient. In one aspect, the dimerization molecule is administered one day after the dimerization switch composition has been administered to the patient. In one aspect, the dimerization molecule is administered 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the dimerization switch composition has been administered to the patient. In an aspect the dimerization molecule is administered after administration of the dimerization switch composition, e.g., on or after 1, 2, 3, 4, 5, 6, 7, 8, 9, 10, 11, 12, 16, 17, 18, 19, 20, 21, 22, or 23 hours, or on or after 1, 2, 3, 4, 5, 6, 7 or 8 days, after administration of the dimerization switch composition. In one aspect, the dimerization molecule is administered more than once to the after the dimerization switch composition has been administered to the patient, e.g., based on a dosing schedule tailored for the patient, e.g., administration of the dimerization molecule on a bi-weekly, weekly, monthly, 6-monthly, yearly basis. In an aspect, dosing of the dimerization molecule will be daily, every other day, twice a week, or weekly, but in some aspects will not exceed 5 mg, 10 mg, 15 mg, 20 mg, 30 mg, 40 mg, or 50 mg, weekly. In an aspect, the dimerization molecule is dosed continuously, e.g. by use of a pump, e.g., a wearable pump. In an aspect continuous administration lasts for at least 4 hours, 12 hours, 24 hours, 2 days, 3 days, 4 days or 5 days. In an aspect, a FKBP-FRB heterodimerization molecule, e.g., rapamycin, or a rapalog, e.g., AP21967 or RAD001, is administered at a dose of no greater than about 0.5 mg in a 24 hr period.

In an aspect a dimerization molecule is administered at the same time, e.g., on the same day, as the administration of the dimerization switch composition.

Dosages of dimerization molecules depend on the type of dimerization molecule being used and the PK properties of the individual dimerization molecules.

Also provided herein are compositions comprising a FKBP-FRB heterodimerization molecule, e.g., rapamycin, or a rapalog, e.g., AP21967 or RAD001 at a concentration of about 0.005-1.5 mg, about 0.005-1.5 mg, about 0.01-1 mg, about 0.01-0.7 mg, about 0.01-0.5 mg, or about 0.1-0.5 mg. In a further aspect the present invention provides compositions comprising a FKBP-FRB heterodimerization molecule, e.g., rapamycin, or a rapalog, e.g., AP21967 or RAD001 at a concentration of 0.005-1.5 mg, 0.005-1.5 mg, 0.01-1 mg, 0.01-0.7 mg, 0.01-0.5 mg, or 0.1-0.5 mg. More particularly, in one aspect, the invention provides compositions comprising a FKBP-FRB heterodimerization molecule, e.g., rapamycin, or a rapalog, e.g., AP21967 or RAD001 at a dose of about 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, or 1.0 mg. In one aspect, the FKBP-FRB heterodimerization molecule, e.g., rapamycin, or a rapalog, e.g., AP21967 or RAD001 is at a dose of 0.5 mg or less. In a still further aspect, a FKBP-FRB heterodimerization molecule, e.g., rapamycin, or a rapalog, e.g., AP21967 or RAD001 is at a dose of about 0.5 mg. In a further aspect, the invention provides compositions comprising a FKBP-FRB heterodimerization molecule, e.g., rapamycin, or a rapalog, e.g., AP21967 or RAD001 at a dose of 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, or 1.0 mg. In one aspect, a FKBP-FRB heterodimerization molecule, e.g., rapamycin, or a rapalog, e.g., AP21967 or RAD001 is at a dose of 0.5 mg or less. In a still further aspect, a FKBP-FRB heterodimerization molecule, e.g., rapamycin, or a rapalog, e.g., AP21967 or RAD001 is at a dose of 0.5 mg. In a further aspect, the invention relates to compositions comprising an rapamycin, or a rapamycin analog, that is not RAD001, in an amount that is bioequivalent to the specific amounts or doses specified for RAD001. In a further aspect, the invention relates to compositions comprising a FKBP-FRB heterodimerization molecule, e.g., rapamycin, or a rapalog, e.g., AP21967 or RADOOlin an amount sufficient to promote RCART activation following target engagement, as measured by NFAT activation, tumor cell killing or cytokine production. In an aspect the dose of a FKBP-FRB heterodimerization molecule, e.g., rapamycin, or a rapalog, e.g., AP21967 or RAD001 is not immunsuppressive. In an aspect a dose provided here is designed to produce only partial or minimal inhibition of mTOR activity.

Also within the invention are unit dosage forms of a heterodimerization molecule, e.g., rapamycin, or a rapalog, e.g., AP21967 or RAD001, that contain 25%, 50%, 100%, 150% or 200% of any daily dosage referred to herein.

A FKBP-FRB heterodimerization molecule, e.g., rapamycin, or a rapalog, e.g., AP21967 or RAD001, can be administered at a dose that results in a therapeutic effect.

In an aspect, rapamycin, or a rapalog, e.g., AP21967 or RAD001, is administered at a dose of about 0.005-1.5 mg daily, about 0.01-1 mg daily, about 0.01-0.7 mg daily, about 0.01-0.5 mg daily, or about 0.1-0.5 mg daily.

In an aspect, rapamycin, or a rapalog, e.g., AP21967 or RAD001, is administered at a dose of 0.005-1.5 mg daily, 0.005-1.5 mg daily, 0.01-1 mg daily, 0.01-0.7 mg daily, 0.01-0.5 mg daily, or 0.1-0.5 mg daily.

In an aspect, rapamycin, or a rapalog, e.g., AP21967 or RAD001, is administered at a dose of about: 0.005 mg daily, 0.006 mg daily, 0.007 mg daily, 0.008 mg daily, 0.009 mg daily, 0.01 mg daily, 0.02 mg daily, 0.03 mg daily, 0.04 mg daily, 0.05 mg daily, 0.06 mg daily, 0.07 mg daily, 0.08 mg daily, 0.09 mg daily, 0.1 mg daily, 0.2 mg daily, 0.3 mg daily, 0.4 mg daily, 0.5 mg daily, 0.6 mg daily, 0.7 mg daily, 0.8 mg daily, 0.9 mg daily, or 1.0 mg daily.

In an aspect, rapamycin, or a rapalog, e.g., AP21967 or RAD001, is administered at a dose of 0.5 mg daily, or less than 0.5 mg daily.

In an aspect, rapamycin, or a rapalog, e.g., AP21967 or RAD001, is administered at a dose of about 0.1-20 mg weekly, about 0.5-15 mg weekly, about 1-10 mg weekly, or about 3-7 mg weekly.

In an aspect, rapamycin, or a rapalog, e.g., AP21967 or RAD001, is administered at a dose of 0.1-20 mg weekly, 0.5-15 mg weekly, 1-10 mg weekly, or 3-7 mg weekly.

In an aspect, rapamycin, or a rapalog, e.g., AP21967 or RAD001, is administered at a dose of no greater than about: 0.7 mg in a 24 hour period; 0.5 mg in a 24 hour period. In some aspects, rapamycin, or a rapalog, e.g., AP21967 or RAD001, can be administered at a dose of or 0.5 mg, or less daily. In some aspects, rapamycin, or a rapalog, e.g., AP21967 or RAD001,01 can be administered at a dose of 0.5 mg daily.

In an aspect, rapamycin, or a rapalog, e.g., AP21967 or RAD001, is administered at a dose of about: 0.1 mg weekly, 0.2 mg weekly, 0.3 mg weekly, 0.4 mg weekly, 0.5 mg weekly, 0.6 mg weekly, 0.7 mg weekly, 0.8 mg weekly, 0.9 mg weekly, 1 mg weekly, 2 mg weekly, 3 mg weekly, 4 mg weekly, 5 mg weekly, 6 mg weekly, 7 mg weekly,8 mg weekly,9 mg weekly, 10 mg weekly, 11 mg weekly, 12 mg weekly, 13 mg weekly, 14 mg weekly, 15 mg weekly, 16 mg weekly, 17 mg weekly, 18 mg weekly, 19 mg weekly, or 20 mg weekly.

In an aspect, the invention can utilize an FKBP-FRB heterodimerization molecule other than RAD001 in an amount that is bioequivalent, in terms of its ability to activate a RCAR, to the specific amounts or doses specified for RAD001.

In an aspect, rapamycin, or a rapalog, e.g., AP21967 or RAD001, is administered at a dosage of about: 30 pM to 4 nM; 50 pM to 2 nM; 100 pM to 1.5 nM; 200 pM to 1 nM; 300 pM to 500 pM; 50 pM to 2 nM; 100 pM to 1.5 nM; 200 pM to 1 nM; or 300 pM to 500 pM.

In an aspect, rapamycin, or a rapalog, e.g., AP21967 or RAD001, is administered at a dosage of about: 30 pM, 40 pM, 50 pM, 60 pM, 70 pM, 80 pM, 90 pM, 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 1.5 nM, 2 nM, 2.5 nM, 3 nM, 3.5 nM, or 4 nM.

In an aspect, rapamycin, or a rapalog, e.g., AP21967 or RAD001, is administered to a subject at a dosage that provides a target trough level. As used herein, the term “trough level” refers to the concentration of a drug in plasma just before the next dose, or the minimum drug concentration between two doses. In an aspect, the trough level is significantly lower than trough levels associated with dosing regimens used in organ transplant and cancer patients. In an aspect rapamycin, or a rapalog, e.g., AP21967 or RAD001, is administered to result in a trough level that is less than ½, ¼, 1/10, or 1/20 of the trough level that results in immunosuppression or an anticancer effect. In an aspect rapamycin, or a rapalog, e.g., AP21967 or RAD001, is administered to result in a trough level that is less than ½, ¼, 1/10, or 1/20 of the trough level provided on the FDA approved packaging insert for use in immunosuppression or an anticancer indications.

In an aspect, a heterodimerization molecule, e.g., rapamycin, or a rapalog, e.g., AP21967 or RAD001, is administered in sufficient amounts to provide a trough level in a selected range. In an aspect the range is selected from between: 0.1 and 4.9 ng/ml; 2.4 and 4.9 ng/ml; about 0.1 and 2.4 ng/ml; about 0.1 and 1.5 ng/ml.

In an aspect, a heterodimerization molecule, e.g., rapamycin, or a rapalog, e.g., AP21967 or RAD001, is administered in sufficient amounts to provide a trough level of about: is 0.1 ng/ml; 0.2 ng/ml; 0.3 ng/ml; 0.4 ng/ml; 0.5 ng/ml; 0.6 ng/ml; 0.7 ng/ml; 0.8 ng/ml; 0.9 ng/ml; 1.0 ng/ml; 1.1 ng/ml; 1.2 ng/ml; 1.3 ng/ml; 1.4 ng/ml; and 1.5 ng/ml.

In an aspect, a heterodimerization molecule, e.g., rapamycin, or a rapalog, e.g., AP21967 or RAD001, is administered in sufficient amounts to provide a trough level of less than: 5 ng/ml. 2.5 ng/ml; 2 ng/ml; 1.9 ng/ml; 1.8 ng/ml; 1.7 ng/ml; 1.6 ng/ml; 1.5 ng/ml; 1.4 ng/ml; 1.3 ng/ml, 1.2 ng/ml; 1.1 ng/ml; 1.0 ng/ml; 0.9 ng/ml; 0.8 ng/ml; 0.7 ng/ml; 0.6 ng/ml; 0.5 ng/ml; 0.4 ng/ml; 0.3 ng/ml; 0.2 ng/ml; or 0.1 ng/ml.

Also within the invention are unit dosage forms of a heterodimerization molecule, e.g., rapamycin, or a rapalog, e.g., AP21967 or RAD001, that contain any daily dosage referred to herein.

The present invention provides compositions and methods for the treatment of a variety of diseases and disorders. In some aspects, the disease or disorder is a disease or disorder that is associated with abberant gene expression. In some aspects, the disease or disorder is a genetic disorder, e.g., a genetic disorder as described above in the section entitled USES FOR GENE EDITING DIMERIZATION SWITCH-CONTAINING GENE EDITING SYSTEMS. In some aspects, the disease or disorder is a lysosomal storage disorder, e.g., a lysosomal storage disorder described above in the section entitled USES FOR GENE EDITING DIMERIZATION SWITCH-CONTAINING GENE EDITING SYSTEMS.

In other aspects, the present invention provides compositions and methods for the treatment of a subject in need thereof of heart, lung, combined heart lung, liver, kidney, pancreatic, skin or corneal transplants, including, but not limited to, allograft rejection or xenograft rejection, and for the prevention of graft versus host disease, such as following bone marrow transplant, and organ transplant associated arteriosclerosis.

The invention also provides compositions and methods for the treatment, prevention, or amelioration of autoimmune disease and of inflammatory conditions, in particular inflammatory conditions with an aetiology including an autoimmune component such as arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankylosing spondylitis, Reiter syndrome, reactive arthritis, psoriatic arthritis, juvenile idiopathic arthritis and enterophathis arthritis, enthesitis, hypersensitivity (including both airways hypersensitivity and dermal hypersensitivity) and allergies. Specific auto immune diseases for which antibodies of the disclosure may be employed include autoimmune haematological disorders (including e.g. hemolytic anaemia, aplastic anaemia, pure red cell anaemia and idiopathic thrombocytopenia), systemic lupus erythematosus (SLE), lupus nephritis, inflammatory muscle diseases (dermatomyosytis), periodontitis, polychondritis, scleroderma, Wegener granulomatosis, dermatomyositis, chronic active hepatitis, myasthenia gravis, psoriasis, Steven Johnson syndrome, idiopathic sprue, autoimmune inflammatory bowel disease (including e.g. ulcerative colitis, Crohn's disease and irritable bowel syndrome), endocrine ophthalmopathy, Graves' disease, sarcoidosis, multiple sclerosis, systemic sclerosis, fibrotic diseases, primary biliary cirrhosis, juvenile diabetes (diabetes mellitus type I), uveitis, keratoconjunctivitis sicca and vernal keratoconjunctivitis, interstitial lung fibrosis, periprosthetic osteolysis, glomerulonephritis (with and without nephrotic syndrome, e.g. including idiopathic nephrotic syndrome or minimal change nephropathy), multiple myeloma other types of tumors, inflammatory disease of skin and cornea, myositis, loosening of bone implants, metabolic disorders, (such as obesity, atherosclerosis and other cardiovascular diseases including dilated cardiomyopathy, myocarditis, diabetes mellitus type II, and dyslipidemia), and autoimmune thyroid diseases (including Hashimoto thyroiditis), small and medium vessel primary vasculitis, large vessel vasculitides including giant cell arteritis, hidradenitis suppurativa, neuromyelitis optica, Sjögren's syndrome, Behcet's disease, atopic and contact dermatitis, bronchiolitis, inflammatory muscle diseases, autoimmune peripheral neurophaties, immunological renal, hepatic and thyroid diseases, inflammation and atherothrombosis, autoinflammatory fever syndromes, immunohematological disorders, and bullous diseases of the skin and mucous membranes. Anatomically, uveitis can be anterior, intermediate, posterior, or pan-uveitis. It can be chronic or acute. The etiology of uveitis can be autoimmune or non-infectious, infectious, associated with systemic disease, or a white-dot syndrome.

The present invention also provides compositions and methods for the treatment, prevention, or amelioration of asthma, bronchitis, bronchiolitis, idiopathic interstitial pneumonias, pneumoconiosis, pulmonary emphysema, and other obstructive or inflammatory diseases of the airways.

The present invention also provides compositions and methods for treating diseases of bone metabolism including osteoarthritis, osteoporosis and other inflammatory arthritis, and bone loss in general, including age-related bone loss, and in particular periodontal disease.

In addition, the present invention provides compositions and methods for treating chronic candidiasis and other chronic fungal diseases, as well as complications of infections with parasites, and complications of smoking are considered to be promising avenues of treatment, as well as viral infection and complications of viral infection (e.g., HIV infection).

The present invention also provides compositions and methods for treating breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Generation and Characterization of a Mutant FRB Switch Domain

In this example, mutation of the residues involved in binding between the switch domains, e.g., FRB or FKBP, with the dimerization molecule was performed to identify switch domains with enhanced interaction with the dimerization molecule. Libraries of candidate mutant FKBP and FRB switch domains were generated and screened as described herein. Mutant FKBP or FRB with increased affinity and/or which enhance formation of a complex between the mutant switch domain, a second switch domain (e.g., a FRB derived switch domain or a FKBP derived switch domain), and a dimerization molecule, rapamycin, or a rapalog, e.g., RAD001 allows the use of circulating concentrations of the dimerization molecule, e.g., RAD001, which are less than the concentrations used to mediate immunosuppression.

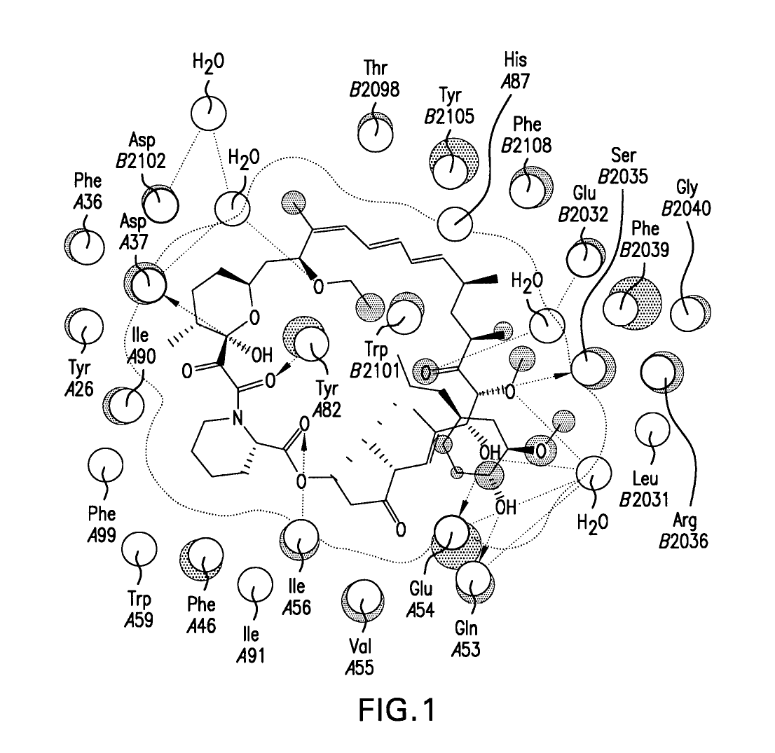

The interface between FKBP, FRB, and rapamycin is clearly defined allowing for inspection of the FRB/rapamycin and FRB/FKBP interface. In the 2.2 Å x-ray structure of the ternary FKBP/FRB/rapamycin complex, FRB residues Leu2031, Glu2032, Ser2035, Arg2036, Phe2039, Gly2040, Thr2098, Trp2101, Tyr2015, and Phe2108 make 38 direct contacts with rapamycin and FRB residues Arg2042 and Asp2102 make water mediated contacts with the compound (4). FIG. 1 shows the rapamycin interaction with FKBP and FRB which were determined in the x-ray structure of the ternary complex, RCSB code 2FAP, generated using the Molecular Operating Environment (MOE) (5). The FRB molecule is chain B in the structure.

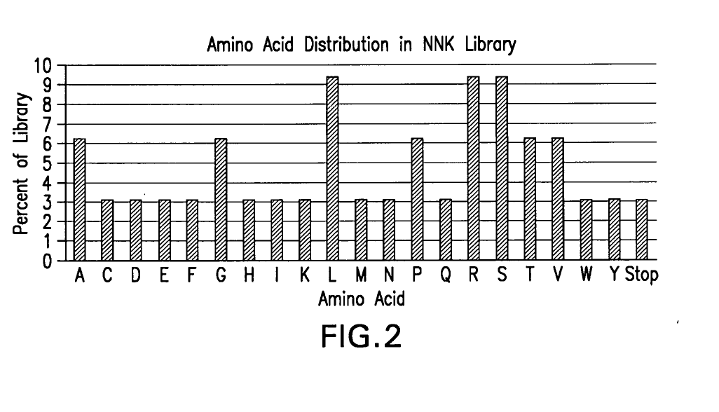

The FRB residues chosen for mutation included: L2031, E2032, S2035, R2036, F2039, G2040, T2098, W2101, D2102, Y2105, and F2108. Each point mutant library was generated by randomizing the codon at the desired position using an NNK library, where N can be adenine (A), cytosine (C), guanine (G), or thymine (T), and K can be guanine (G) or thymine (T). Table 13 shows the codon distribution of an NNK library and the corresponding amino acids. FIG. 2 shows the distributions of the amino acids produced from the codons in the NNK library, ranging from a low of 3.1% to a high 9.4%. Each point mutant library was cloned into the pNAT43 vector with a N-terminal histidine tag. SEQ ID NOs: 36-46 give the amino acid composition of each point mutant library, where X indicates the position of the NNK library. The DNA for each library was transformed into Acella chemically competent E. coli, plated onto 100 mm LB agar plates with 50 μg/mL kanamycin sulfate, and incubated overnight at 37° C. 94 colonies from each library plate were transferred to Costar 2 mL pyramidal bottom 96-well plates with 1 mL of ZYP-5052 auto induction medium containing 75 μg/mL kanamycin sulfate. The plates were incubated for 40 hours at 800 rpm at 30° C. in a micro plate incubator.

The candidate FRB clones were isolated as follows. First, the cells were lysed. The cells were pelleted by centrifugation at 2,000×g at 4° C. for 30 minutes. The supernatant was discarded and the cell pellets were stored at −80° C. The 96-well plates containing the cell pellets were removed from storage at −80° C. and thawed at room temperature for 1 hour. 0.5 mL of 50 mM HEPES pH 7.5, 150 mM NaCl, 5 mM EDTA, 0.25% (v/v) Triton X-100, 2.5 mg/mL lysozyme were added to each well. The pellets were resuspended by pipetting 180 μL, 60 times. The samples were incubated at room temperature for 0.1 to 1 hour. 0.5 mL of 50 mM HEPES pH 7.5, 150 mM NaCl, 20 mM CaCl₂, 20 mM MgCl₂, 0.5 mg/mL DNase I were added to each well. The samples were mixed by pipetting 180 μL, 10 times. The plates were incubated for 30 minutes at room temperature. The lysed cells were pelleted by centrifugation at 2,000×g at 4° C. for 30 minutes. The supernatant was discarded from each plate by inversion followed by gentle tapping. The plates were stored overnight at −80° C.

Next, the stored lysates were processed by affinity purification to isolate the mutant FRB as follows. The following morning, the plates were removed from storage at −80° C. and thawed at room temperature for 1 hour. 0.7 mL of 50 mM HEPES, 500 mM NaCl, 5 mM TCEP, 5% (v/v) Triton X-100, pH 7.5 were added to each well. The pellets were resuspended by pipetting 180 μL 50 times, followed by a 1 hour incubation at room temperature. The plates were centrifuged for 30 minutes at 2,000×g at 4° C. and the supernatant for each was discarded. 0.5 mL of 50 mM HEPES pH 7.5, 1 mM TCEP, 60% ethanol were added to each well. The pellets were resuspended by pipetting 180 μL, 50 times, followed by a 1 hour incubation at room temperature. The plates were centrifuged for 30 minutes at 2,000×g at 4° C. and the supernatant for each was discarded. 0.5 mL of 50 mM HEPES pH 7.5, 500 mM NaCl, 1 mM TCEP, 8 M urea were added to each well. The pellets were resuspended by pipetting 180 μL 50 times and incubated overnight at room temperature. The following morning, the samples were transferred to 20 μm fritted 96-well plates. The samples were filtered through the plates into new 2 mL Costar 96-well plates by centrifugation for 5 minutes at 1,500×g at 4° C. A 25% slurry of Ni Sepharose 6 Fast Flow resin in 50 mM HEPES pH 7.5, 500 mM NaCl, 1 mM TCEP, 8 M urea was prepared. 100 μL of slurry, 25 μL of resin, were added to each well. The resin was incubated with the samples for 1 hour at room temperature. The resin was then transferred to 20 μm fritted 96-well plates and the column flow-through was removed by vacuum. 500 μL of 50 mM HEPES pH 7.5, 150 mM NaCl, 1 mM TCEP, 4 M urea was added to each well, incubated for 5 minutes, and removed by vacuum. 500 μL of 50 mM HEPES pH 7.5, 150 mM NaCl, 1 mM TCEP, 2 M urea was added to each well, incubated for 5 minutes, and removed by vacuum. 500 μL of 50 mM HEPES pH 7.5, 150 mM NaCl, 1 mM TCEP, 1 M urea was added to each well, incubated for 5 minutes, and removed by vacuum. 500 μL of 50 mM HEPES pH 7.5, 150 mM NaCl, 1 mM TCEP, 25 mM imidazole was added to each well, incubated for 5 minutes, and removed by vacuum. 200 μL of 50 mM HEPES pH 7.5, 150 mM NaCl, 1 mM TCEP, 500 mM imidazole was added to each and incubated for 5 minutes. The bound protein was eluted by centrifugation for 2 minutes at 500×g at 4° C. into a new 300 μL BD Falcon 96-well plate. The protein concentration in mg/mL for each well was measured using the Bradford assay with BSA as the standard. The protein concentrations were converted to μM by using the molecular weight for wild type FRB. The point mutant libraries had expression in a least 50% of the wells except for FRB D2102, which was 47%. FIG. 3A shows the expression levels of each library and FIG. 3B shows the average concentration for the expressing wells.

The inhibition for each well expressing protein for each library was calculated by using the well known to contain no protein as blank measurements. For each library plate, the average for the blank wells was calculated. Expressing wells with values greater than the average for the blank wells were defined to have 0% inhibition. The percent inhibition for wells with values less than or equal to the average for the blank wells was calculated by subtracting the average for the blank wells from the well value, dividing by −1 multiplied by the average for the blank wells, and multiplying by 100. When the well value was 0, there was 100% inhibition and when the well value was equal to the average of the blank wells, there was 0% inhibition. Wells with inhibition greater than or equal to 75% were chosen for re-array. Table 6 shows the number of wells selected for each library and the number of wells expected to be wild type FRB. 320 out of 1034 wells were chosen, 31.3%. The selected wells were grown, purified, and analyzed as described. The DNA for each of the selected wells was sequenced to identify the individual mutations. The protein concentration for each of the mutants was assessed by the Bradford assay. The activity of each mutant was compared with the ability of wild type FRB to bind to everolimus, e.g., RAD001, in multiple assay formats.

TABLE 6
Wells selected for retesting for each point mutant
library in the initial screen

| | Wells | Expected Wild |
|---|---|---|
| Library | Selected | Type Wells |
| L2031 | 41 | 9 |
| E2032 | 82 | 3 |
| S2035 | 33 | 9 |
| R2036 | 9 | 9 |
| F2039 | 15 | 3 |
| G2040 | 49 | 6 |
| T2098 | 57 | 6 |
| W2101 | 1 | 3 |
| D2102 | 11 | 3 |
| Y2105 | 6 | 3 |
| F2108 | 16 | 3 |

For the competition assay, FRB mutations of interest are ranked compared to wild type FRB. Unlabeled FRB proteins of interest (SEQ ID NOs: 48-52) and unlabeled wild type FRB (SEQ ID NO: 47) were serial diluted 1:3 from a starting final concentration of between 0.9 and 4 uM dependent upon expression and added in solution with 30 nM (final) wild type Flag-FRB (SEQ ID NO: 53) and 30 nM (final) biotinylated wild-type FKBP (SEQ ID NO: 59) in the presence of 60 nM (final) everolimus in a 96 well ½ surface flat-bottom plate (PerkinElmer). All dilutions were made in 1×AlphaLISA Immunoassay buffer (PerkinElmer). The plate was incubated for one hour at room temperature with mild shaking. Anti-Flag acceptor beads (PerkinElmer) were then added at 10 ug/ml final concentration and incubated for one hour at room temperature with mild shaking. Streptavidin donor beads (PerkinElmer), were then added at a final concentration of 40 ug/ml and the plate was protected from light for a 30 minute room temperature incubation with mild shaking. The plate was then read on the PerkinElmer EnVision Multiplate reader equipped with the Alpha Module using excitation of 680 nm and a 570 nm Emission filter. The EC50s of each FRB sequence from the competition assay are shown in Table 7 in comparison to WT FRB analyzed in the same plate. Single point mutations E2032L (SEQ ID NO: 49) and E2032I (SEQ ID NO: 48) were approximately 2-fold better than wild type (FIGS. 4A and 4B; T2098L (SEQ ID NO: 50) was 3-fold improved (FIG. 4C). FRB proteins incorporating mutations at both sites (SEQ ID NOs: 51 and 52) demonstrated 5-fold relative improvement (FIGS. 4D and 4E).

TABLE 7
EC50 Values from Competition Assay

| Mutation | Ec50 (nM) |
|---|---|
| Wild type FRB (1) | 23.33 |
| E2032L | 12.94 |
| E2032I | 16.61 |
| T2098L | 8.255 |
| Wild type FRB (2) | 42.62 |
| E2032L, T2098L | 8.047 |
| E2032I, T2098L | 7.138 |

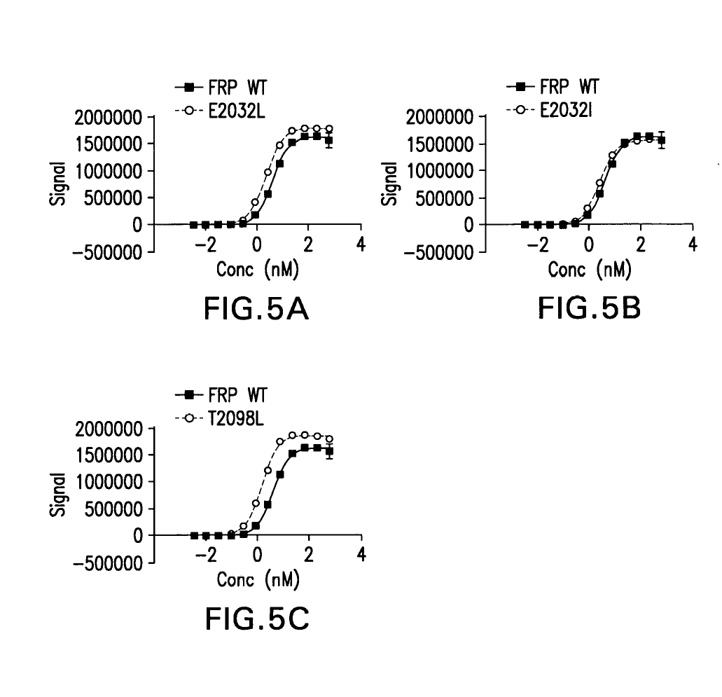

FRB mutations were also ranked in an alternative assay format. Briefly, FRB proteins incorporating single and double mutations (SEQ ID NO: 54-58) were produced as FLAG tagged constructs in E. coli as described previously. 30 nM (final) of biotinylated FKBP (SEQ ID NO: 59) and each FLAG FRB protein were combined in the presence of everolimus serial diluted 1:3 from a starting final concentration of 600 nM into a 96 well ½ surface flat-bottom plate (PerkinElmer) and incubated for one hour at room temperature. All dilutions were made in 1× AlphaLISA Immunoassay buffer (PerkinElmer). Anti-Flag acceptor beads (PerkinElmer) were added at 10 ug/ml final concentration and incubated for one hour at room temperature. Streptavidin donor beads (PerkinElmer), were then added at a final concentration of 40 ug/ml and the plate was protected from light and incubated for 30 minutes at room temperature. The plate was then read on the PerkinElmer EnVision Multiplate reader equipped with the Alpha Module using excitation of 680 nm and a 570 nm Emission filter. The EC50s of each FRB sequence from this assay are shown in Table 8. Single point mutations E2032I (SEQ ID NO: 54) and E2032L (SEQ ID NO: 55) were approximately 1.5-2-fold better than wild type (FIGS. 5A and 5B); T2098L (SEQ ID NO: 56) was 3-fold improved (FIG. 5C). Flag-tagged FRB proteins which incorporated the double mutations (SEQ ID NO: 57 and 32358 demonstrated limited dynamic range in this assay and therefore could not be evaluated.

TABLE 8
EC50 Values from Direct Binding Assay

| Mutation | Ec50 (nM) |
|---|---|
| Wild type FRB | 3.899 |
| E2032L | 2.146 |
| E2032I | 2.461 |
| T2098L | 1.442 |

TABLE 9
Sequences of candidate mutant FRB and constructs
used in binding assays. Tag sequences, e.g., His-
and avi-tags are highlighted (N-Terminal) and
amino acids associated with the cloning process
(C-terminal) are underlined without bold.

| | | SEQ |
|---|---|---|
| | | ID |
| Name | Amino Acid Sequence | NO: |
| L2031 library | MGHHHHHHHHGSASRILWHEMWHEGXEEASRLYFGERNVKGMFEVLEPLHAMMER | 36 |
| | GPQTLKETSFNQAYGRDLMEAQEWCRKYMKSGNVKDLTQAWDLYYHVFRRISKTS | |
| E2032 library | MGHHHHHHHHGSASRILWHEMWHEGLXEASRLYFGERNVKGMFEVLEPLHAMMER | 37 |
| | GPQTLKETSFNQAYGRDLMEAQEWCRKYMKSGNVKDLTQAWDLYYHVFRRISKTS | |
| S2035 library | MGHHHHHHHHGSASRILWHEMWHEGLEEAXRLYFGERNVKGMFEVLEPLHAMMER | 38 |
| | GPQTLKETSFNQAYGRDLMEAQEWCRKYMKSGNVKDLTQAWDLYYHVFRRISKTS | |
| R2036 library | MGHHHHHHHHGSASRILWHEMWHEGLEEASXLYFGERNVKGMFEVLEPLHAMMER | 39 |
| | GPQTLKETSFNQAYGRDLMEAQEWCRKYMKSGNVKDLTQAWDLYYHVFRRISKTS | |
| F2039 library | MGHHHHHHHHGSASRILWHEMWHEGLEEASRLYXGERNVKGMFEVLEPLHAMMER | 40 |
| | GPQTLKETSFNQAYGRDLMEAQEWCRKYMKSGNVKDLTQAWDLYYHVFRRISKTS | |
| G2040 library | MGHHHHHHHHGSASRILWHEMWHEGLEEASRLYFXERNVKGMFEVLEPLHAMMER | 41 |
| | GPQTLKETSFNQAYGRDLMEAQEWCRKYMKSGNVKDLTQAWDLYYHVFRRISKTS | |
| T2098 library | MGHHHHHHHHGSASRILWHEMWHEGLEEASRLYFGERNVKGMFEVLEPLHAMMER | 42 |
| | GPQTLKETSFNQAYGRDLMEAQEWCRKYMKSGNVKDLXQAWDLYYHVFRRISKTS | |
| W2101 library | MGHHHHHHHHGSASRILWHEMWHEGLEEASRLYFGERNVKGMFEVLEPLHAMMER | 43 |
| | GPQTLKETSFNQAYGRDLMEAQEWCRKYMKSGNVKDLTQAXDLYYHVFRRISKTS | |
| D2102 library | MGHHHHHHHHGSASRILWHEMWHEGLEEASRLYFGERNVKGMFEVLEPLHAMMER | 44 |
| | GPQTLKETSFNQAYGRDLMEAQEWCRKYMKSGNVKDLTQAWXLYYHVFRRISKTS | |
| Y2105 library | MGHHHHHHHHGSASRILWHEMWHEGLEEASRLYFGERNVKGMFEVLEPLHAMMER | 45 |
| | GPQTLKETSFNQAYGRDLMEAQEWCRKYMKSGNVKDLTQAWDLYXHVERRISKTS | |
| F2108 library | MGHHHHHHHHGSASRILWHEMWHEGLEEASRLYFGERNVKGMFEVLEPLHAMMER | 46 |
| | GPQTLKETSFNQAYGRDLMEAQEWCRKYMKSGNVKDLTQAWDLYYHVXRRISKTS | |
| His-FRB | MGHHHHHHHHGSASRILWHEMWHEGLEEASRLYFGERNVKGMFEVLEPLHAMMER | 47 |
| (wild-type FRB) | GPQTLKETSFNQAYGRDLMEAQEWCRKYMKSGNVKDLTQAWDLYYHVERRISKTS | |
| His-FRB | MGHHHHHHHHGSASRILWHEMWHEGLIEASRLYFGERNVKGMFEVLEPLHAMMER | 48 |
| E2032I | GPQTLKETSFNQAYGRDLMEAQEWCRKYMKSGNVKDLTQAWDLYYHVERRISKTS | |
| His-FRB | MGHHHHHHHHGSASRILWHEMWHEGLLEASRLYFGERNVKGMFEVLEPLHAMMER | 49 |
| E2032L | GPQTLKETSFNQAYGRDLMEAQEWCRKYMKSGNVKDLTQAWDLYYHVERRISKTS | |
| His-FRB T | MGHHHHHHHHGSASRILWHEMWHEGLEEASRLYFGERNVKGMFEVLEPLHAMMER | 50 |
| 2098L | GPQTLKETSFNQAYGRDLMEAQEWCRKYMKSGNVKDLLQAWDLYYHVERRISKTS | |
| His-FRB | MGHHHHHHHHGSASRILWHEMWHEGLIEASRLYFGERNVKGMFEVLEPLHAMMER | 51 |
| E2032I, T2098L | GPQTLKETSFNQAYGRDLMEAQEWCRKYMKSGNVKDLLQAWDLYYHVERRISKTS | |
| His-FRB | MGHHHHHHHHGSASRILWHEMWHEGLLEASRLYFGERNVKGMFEVLEPLHAMMER | 52 |
| E2032L, T2098L | GPQTLKETSFNQAYGRDLMEAQEWCRKYMKSGNVKDLLQAWDLYYHVERRISKTS | |
| His-FLAG-FRB | MGHHHHHHHHGSDYKDDDDKGSASRILWHEMWHEGLEEASRLYFGERNVKGMFEV | 53 |
| (wild-type FRB) | LEPLHAMMERGPQTLKETSFNQAYGRDLMEAQEWCRKYMKSGNVKDLTQAWDLYY | |
| | HVERRISKTS | |
| His-FLAG-FRB | MGHHHHHHHHGSDYKDDDDKGSASRILWHEMWHEGLIEASRLYFGERNVKGMFEV | 54 |
| E2032I | LEPLHAMMERGPQTLKETSFNQAYGRDLMEAQEWCRKYMKSGNVKDLTQAWDLYY | |
| | HVERRISKTS | |
| His-FLAG-FRB | MGHHHHHHHHGSDYKDDDDKGSASRILWHEMWHEGLLEASRLYFGERNVKGMFEV | 55 |
| E2032L | LEPLHAMMERGPQTLKETSFNQAYGRDLMEAQEWCRKYMKSGNVKDLTQAWDLYY | |
| | HVERRISKTS | |
| His-FLAG-FRB | MGHHHHHHHHGSDYKDDDDKGSASRILWHEMWHEGLEEASRLYFGERNVKGMFEV | 56 |
| T2098L | LEPLHAMMERGPQTLKETSFNQAYGRDLMEAQEWCRKYMKSGNVKDLLQAWDLYY | |
| | HVERRISKTS | |
| His-FLAG-FRB | MGHHHHHHHHGSDYKDDDDKGSASRILWHEMWHEGLIEASRLYFGERNVKGMFEV | 57 |
| E2032I, T2098L | LEPLHAMMERGPQTLKETSFNQAYGRDLMEAQEWCRKYMKSGNVKDLLQAWDLYY | |
| | HVERRISKTS | |
| His-FLAG-FRB | MGHHHHHHHHGSDYKDDDDKGSASRILWHEMWHEGLLEASRLYFGERNVKGMFEV | 58 |
| E2032L, T2098L | LEPLHAMMERGPQTLKETSFNQAYGRDLMEAQEWCRKYMKSGNVKDLLQAWDLYY | |
| | HVERRISKTS | |
| His-Avidin-FKBP | MGHHHHHHHHGSGLNDIFEAQKIEWHEGSGVQVETISPGDGRTFPKRGQTCVVHY | 59 |
| (wild-type FKBP) | TGMLEDGKKFDSSRDRNKPFKFMLGKQEVIRGWEEGVAQMSVGQRAKLTISPDYA | |
| | YGATGHPGIIPPHATLVFDVELLKLE | |

### Example 2: Generation and Characterization of a Mutant FKBP Switch Domain

The interface between FKBP, FRB, and rapamycin is clearly defined allowing for inspection of the FRB/rapamycin and FRB/FKBP interface, as described herein and in Example 1. In the 2.2 Å x-ray structure of the ternary FKBP/FRB/rapamycin complex, FKBP residues Tyr26, Phe36, Asp37, Phe46, Gln53, Glu54, Val55, Ile56, Tyr59, Tyr82, Ile90, Ile91, and Phe99 make 84 direct contacts, while Arg42, Lys44, Pro45, Lys47, Glu54, and His87 make water mediated contacts with the compound (Liang et al. J. Acta Cryst. (1999), D55:736-744). FIG. 1 shows the rapamycin interaction with FKBP and FRB which were determined in the x-ray structure of the ternary complex, RCSB code 2FAP, generated using the Molecular Operating Environment (MOE) (5). The FKBP molecule is chain A in the structure. The FKPB residues chosen for mutation are shown in Table 1 and number by their position in the UniProtKB entry P62942, which shifts the numbering +1 relative to the crystal structure. Each point mutant library was generated by randomizing the codon at the desired position using an NNK library, where N can be adenine (A), cytosine (C), guanine (G), or thymine (T), and K can be guanine (G) or thymine (T). Table 10 shows the codon distribution of an NNK library and the corresponding amino acids. FIG. 2 shows the distributions of the amino acids produced from the codons in the NNK library, ranging from a low of 3.1% to a high 9.4%. Each point mutant library was cloned into the pNAT43 vector with a N-terminal histidine tag.

TABLE 10
Mutation Libraries

| FKBP Residue Number | Wild Type Amino Acid |
|---|---|
| 26 | Tyrosine, Tyr, Y |
| 36 | Phenylalanine, Phe, F |
| 37 | Aspartic Acid, Asp, D |
| 42 | Arginine, Arg, R |
| 46 | Phenylalanine, Phe, F |
| 53 | Glutamine, Gln, Q |
| 54 | Glutamic Acid, Glu, E |
| 55 | Valine, Val, V |
| 56 | Isoleucine, Ile, I |
| 59 | Trptophan, Trp, W |
| 82 | Tyrosine, Tyr, Y |
| 87 | Histidine, His, H |
| 89 | Glycine, Gly, G |
| 90 | Isoleucine, Ile, I |
| 99 | Phenylalanine, Phe, F |

Sequences 60-77 give the amino acid composition of each point mutant library, where X indicates the position of the NNK library. The DNA for each library was transformed into Acella chemically competent E. coli, plated onto 100 mm LB agar plates with 50 μg/mL kanamycin sulfate, and incubated overnight at 37° C. 94 colonies from each library plate were transferred to Costar 2 mL pyramidal bottom 96-well plates with 1 mL of ZYP-5052 auto induction medium containing 75 μg/mL kanamycin sulfate. The plates were incubated for 40 hours at 800 rpm at 30° C. in a micro plate incubator. The cells were pelleted by centrifugation at 2,000×g at 4° C. for 30 minutes. The supernatant was discarded and the cell pellets were stored at −80° C. The 96-well plates containing the cell pellets were removed from storage at −80° C. and thawed at room temperature for 1 hour. 0.5 mL of 50 mM HEPES pH 7.5, 150 mM NaCl, 5 mM EDTA, 0.25% (v/v) Triton X-100, 2.5 mg/mL lysozyme were added to each well. The pellets were resuspended by pipetting 180 μL 60 times. The samples were incubated at room temperature for 0.1 to 1 hour. 0.5 mL of 50 mM HEPES pH 7.5, 150 mM NaCl, 20 mM CaCl₂, 20 mM MgCl₂, 0.5 mg/mL DNase I were added to each well. The samples were mixed by pipetting 180 μL 10 times. The plates were incubated for 30 minutes at room temperature. The lysed cells were pelleted by centrifugation at 2,000×g at 4° C. for 30 minutes. The supernatant was discarded from each plate by inversion followed by gentle tapping. The plates were stored overnight at −80° C. The following morning, the plates were removed from storage at −80° C. and thawed at room temperature for 1 hour. 0.7 mL of 50 mM HEPES, 500 mM NaCl, 5 mM TCEP, 5% (v/v) Triton X-100, pH 7.5 were added to each well. The pellets were resuspended by pipetting 180 μL 50 times, followed by a 1 hour incubation at room temperature. The plates were centrifuged for 30 minutes at 2,000×g at 4° C. and the supernatant for each was discarded. 0.5 mL of 50 mM HEPES pH 7.5, 1 mM TCEP, 60% ethanol were added to each well. The pellets were resuspended by pipetting 180 μL 50 times, followed by a 1 hour incubation at room temperature. The plates were centrifuged for 30 minutes at 2,000×g at 4° C. and the supernatant for each was discarded. 0.5 mL of 50 mM HEPES pH 7.5, 500 mM NaCl, 1 mM TCEP, 8 M urea were added to each well. The pellets were resuspended by pipetting 180 μL 50 times and incubated overnight at room temperature. The following morning, the samples were transferred to 20 μm fritted 96-well plates. The samples were filtered through the plates into new 2 mL Costar 96-well plates by centrifugation for 5 minutes at 1,500×g at 4° C. A 25% slurry of Ni Sepharose 6 Fast Flow resin in 50 mM HEPES pH 7.5, 500 mM NaCl, 1 mM TCEP, 8 M urea was prepared. 100 μL of slurry, 25 μL of resin, were added to each well. The resin was incubated with the samples for 1 hour at room temperature. The resin was then transferred to 20 μm fritted 96-well plates and the column flow-through was removed by vacuum. 500 μL of 50 mM HEPES pH 7.5, 150 mM NaCl, 1 mM TCEP, 4 M urea was added to each well, incubated for 5 minutes, and removed by vacuum. 500 μL of 50 mM HEPES pH 7.5, 150 mM NaCl, 1 mM TCEP, 2 M urea was added to each well, incubated for 5 minutes, and removed by vacuum. 500 μL of 50 mM HEPES pH 7.5, 150 mM NaCl, 1 mM TCEP, 1 M urea was added to each well, incubated for 5 minutes, and removed by vacuum. 500 μL of 50 mM HEPES pH 7.5, 150 mM NaCl, 1 mM TCEP, 25 mM imidazole was added to each well, incubated for 5 minutes, and removed by vacuum. 200 μL of 50 mM HEPES pH 7.5, 150 mM NaCl, 1 mM TCEP, 500 mM imidazole was added to each and incubated for 5 minutes. The bound protein was eluted by centrifugation for 2 minutes at 500×g at 4° C. into a new 300 μL BD Falcon 96-well plate. The protein concentration in mg/mL for each well was measured using the Bradford assay with BSA as the standard. The protein concentrations were converted to μM by using the molecular weight for wild type FKBPB. To screen the libraries unlabeled FKBP neat protein (Seq 1-15) was added in solution with 30 nM (final) biotinylated wild-type FKBP (Seq 17) and 30 nM (final) wild type Flag-FRB (Seq 18) in the presence of 60 nM (final) Everolimus in a 96 well ½ surface flat-bottom plate (PerkinElmer) and incubated for one hour at room temperature. Unlabeled wild type FRBP (Seq 16) was added as a control for each plate. Anti-flag acceptor beads (PerkinElmer) were then added at 10 ug/ml final concentration and incubated for one hour. Streptavidin donor beads (PerkinElmer), were then added at a final concentration of 40 ug/ml and the plate was protected from light for a 30 minute incubation. The plate was then read on the PerkinElmer EnVision Multiplate reader equipped with the Alpha Module using excitation of 680 nm and a 570 nm Emission filter. All dilutions were made in 1×AlphaELISA Immunoassay buffer (PerkinElmer) and all incubations were performed at room temperature with shaking. The inhibition for each well expressing protein for each library was calculated by using the well known to contain no protein as blank measurements. For each library plate, the average for the blank wells was calculated. Expressing wells with values greater than the average for the blank wells were defined to have 0% inhibition. The percent inhibition for wells with values less than or equal to the average for the blank wells was calculated by subtracting the average for the blank wells from the well value, dividing by −1 multiplied by the average for the blank wells, and multiplying by 100. When the well value was 0, there was 100% inhibition and when the well value was equal to the average of the blank wells, there was 0% inhibition. Wells with inhibition greater than or equal to 75% were chosen for rearray. Table 3 shows the number of wells selected for each library and the number of wells expected to be wild type FRB. 320 out of 1034 wells were chosen, 31.3%. The selected wells were grown, purified, and assayed as described. The DNA for each of the selected wells was sequenced to identify the individual mutations. The protein concentration for each of the mutants was assessed by the Bradford assay.

TABLE 11
Sequences of candidate FKBP mutants and constructs used in
binding assays. His-tag, avi- and FLAG-tag sequences
(N-terminal) and amino acids associated with the cloning
process (C-terminal) are underlined without bold.

| | | SEQ |
|---|---|---|
| | | ID |
| Name | Amino Acid Sequence | NO: |
| FKBP Y26 library | MGHHHHHHHHGSGVQVETISPGDGRTFPKRGQTCVVHXTGMLEDGKKFDSSRD | 60 |
| | RNKPFKFMLGKQEVIRGWEEGVAQMSVGQRAKLTISPDYAYGATGHPGIIPPH | |
| | ATLVFDVELLKLE | |
| FKBP F36 library | MGHHHHHHHHGSGVQVETISPGDGRTFPKRGQTCVVHYTGMLEDGKKXDSSRD | 61 |
| | RNKPFKFMLGKQEVIRGWEEGVAQMSVGQRAKLTISPDYAYGATGHPGIIPPH | |
| | ATLVFDVELLKLE | |
| FKBP D37 library | MGHHHHHHHHGSGVQVETISPGDGRTFPKRGQTCVVHYTGMLEDGKKFXSSRD | 62 |
| | RNKPFKFMLGKQEVIRGWEEGVAQMSVGQRAKLTISPDYAYGATGHPGIIPPH | |
| | ATLVFDVELLKLE | |
| FKBP R42 library | MGHHHHHHHHGSGVQVETISPGDGRTFPKRGQTCVVHYTGMLEDGKKFDSSRD | 63 |
| | XNKPFKFMLGKQEVIRGWEEGVAQMSVGQRAKLTISPDYAYGATGHPGIIPPH | |
| | ATLVFDVELLKLE | |
| FKBP F46 library | MGHHHHHHHHGSGVQVETISPGDGRTFPKRGQTCVVHYTGMLEDGKKFDSSRD | 64 |
| | RNKPXKFMLGKQEVIRGWEEGVAQMSVGQRAKLTISPDYAYGATGHPGIIPPH | |
| | ATLVFDVELLKLE | |
| FKBP Q53 library | MGHHHHHHHHGSGVQVETISPGDGRTFPKRGQTCVVHYTGMLEDGKKFDSSRD | 65 |
| | RNKPFKFMLGKXEVIRGWEEGVAQMSVGQRAKLTISPDYAYGATGHPGIIPPH | |
| | ATLVFDVELLKLE | |
| FKBP E54 library | MGHHHHHHHHGSGVQVETISPGDGRTFPKRGQTCVVHYTGMLEDGKKFDSSRD | 66 |
| | RNKPFKFMLGKQXVIRGWEEGVAQMSVGQRAKLTISPDYAYGATGHPGIIPPH | |
| | ATLVFDVELLKLE | |
| FKBP V55 library | MGHHHHHHHHGSGVQVETISPGDGRTFPKRGQTCVVHYTGMLEDGKKFDSSRD | 67 |
| | RNKPFKFMLGKQEXIRGWEEGVAQMSVGQRAKLTISPDYAYGATGHPGIIPPH | |
| | ATLVFDVELLKLE | |
| FKBP I56 library | MGHHHHHHHHGSGVQVETISPGDGRTFPKRGQTCVVHYTGMLEDGKKFDSSRD | 68 |
| | RNKPFKFMLGKQEVXRGWEEGVAQMSVGQRAKLTISPDYAYGATGHPGIIPPH | |
| | ATLVFDVELLKLE | |
| FKBP W59 library | MGHHHHHHHHGSGVQVETISPGDGRTFPKRGQTCVVHYTGMLEDGKKFDSSRD | 69 |
| | RNKPFKFMLGKQEVIRGXEEGVAQMSVGQRAKLTISPDYAYGATGHPGIIPPH | |
| | ATLVFDVELLKLE | |
| FKBP Y82 library | MGHHHHHHHHGSGVQVETISPGDGRTFPKRGQTCVVHYTGMLEDGKKFDSSRD | 70 |
| | RNKPFKFMLGKQEVIRGWEEGVAQMSVGQRAKLTISPDYAXGATGHPGIIPPH | |
| | ATLVFDVELLKLE | |
| FKBP H87 library | MGHHHHHHHHGSGVQVETISPGDGRTFPKRGQTCVVHYTGMLEDGKKFDSSRD | 71 |
| | RNKPFKFMLGKQEVIRGWEEGVAQMSVGQRAKLTISPDYAYGATGXPGIIPPH | |
| | ATLVFDVELLKLE | |
| FKBP G89 library | MGHHHHHHHHGSGVQVETISPGDGRTFPKRGQTCVVHYTGMLEDGKKFDSSRD | 72 |
| | RNKPFKFMLGKQEVIRGWEEGVAQMSVGQRAKLTISPDYAYGATGHPXIIPPH | |
| | ATLVFDVELLKLE | |
| FKBP I90 library | MGHHHHHHHHGSGVQVETISPGDGRTFPKRGQTCVVHYTGMLEDGKKFDSSRD | 73 |
| | RNKPFKFMLGKQEVIRGWEEGVAQMSVGQRAKLTISPDYAYGATGHPGXIPPH | |
| | ATLVFDVELLKLE | |
| FKBP F99 library | MGHHHHHHHHGSGVQVETISPGDGRTFPKRGQTCVVHYTGMLEDGKKFDSSRD | 74 |
| | RNKPFKFMLGKQEVIRGWEEGVAQMSVGQRAKLTISPDYAYGATGHPGIIPPH | |
| | ATLVXDVELLKLE | |
| FKBP Wild type | MGHHHHHHHHGSGVQVETISPGDGRTFPKRGQTCVVHYTGMLEDGKKFDSSRD | 75 |
| | RNKPFKFMLGKQEVIRGWEEGVAQMSVGQRAKLTISPDYAYGATGHPGIIPPH | |
| | ATLVFDVELLKLE | |
| His-Avi-FKBP | MGHHHHHHHHGSGLNDIFEAQKIEWHEGSGVQVETISPGDGRTFPKRGQTCVV | 76 |
| | HYTGMLEDGKKFDSSRDRNKPFKFMLGKQEVIRGWEEGVAQMSVGQRAKLTIS | |
| | PDYAYGATGHPGIIPPHATLVFDVELLKLE | |
| His-FLAG-FRB | MGHHHHHHHHGSDYKDDDDKGSASRILWHEMWHEGLEEASRLYFGERNVKGMF | 77 |
| | EVLEPLHAMMERGPQTLKETSFNQAYGRDLMEAQEWCRKYMKSGNVKDLTQAW | |
| | DLYYHVFRRISKTS | |

### Example 3: Dimerization Switch for Tissue Regeneration and Repair

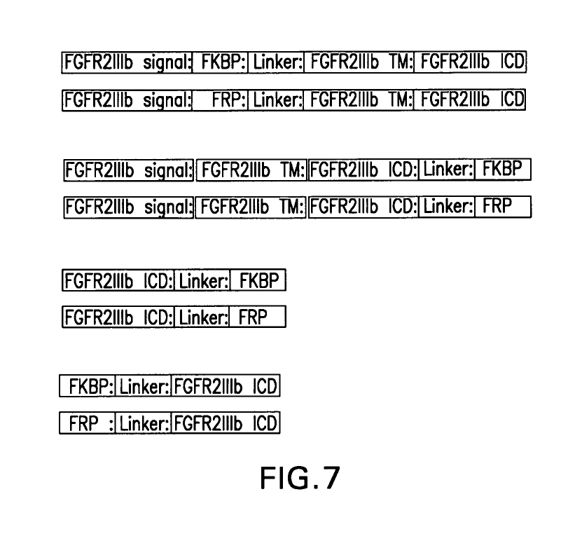

RTK-mediated induction, via homo- or heterodimerization after growth factor binding, of the PI3K/AKT pathway is an important pathway for cell proliferation and tissue repair. Introduction of transgene(s) via targeted/local delivery or cell specific promoters encoding for inducible/switchable homo- or heterodimers may provide an avenue for therapeutic intervention after tissue damage. Addition of the low molecular weight dimerizer provides the mechanism for tunable activity (FIG. 7). Engineering the FKBP-FRP switch for higher affinity will enhance the activity by limiting suppression induced effects of approved rapalogs such as rapamycin and affinitor.

### Liver Repair

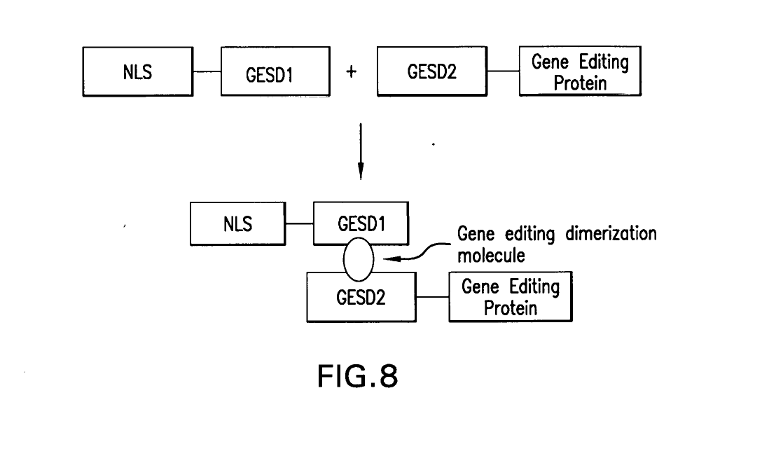

FGFR2IIIb is highly expressed in the liver and integral to cell proliferation. Plasmids encoding FKBP/FRP pairs as fusions with FGFR2IIIb will be synthesized externally as shown in FIG. 8. HepG2, THLE-3, THLE-2 will be used as a surrogate cell lines for primary hepatocytes and will be cultured according to the suppliers recommended conditions. For harvesting of the cells, cells will be detached with accutase and subsequently diluted in media. For each transfection, 1×10⁶ cells will be spun down at 200 g for 10 minutes. One μg of DNA per FKBP construct and one μg of DNA FRP construct will be used per transfection. 100 μl Cell Line Nucleofector Solution X (Lonza) will be added into the tube with DNA constructs. The mixture will be then added to the cells and transferred to the electroporation cuvette. Electroporation will be done under setting EH100 using Amaxa 4D Nucleofector Device. 0.5 ml of growth media will be added immediately after electroporation and the mixture transferred into 9.5 ml growth media. 1×10⁴ cells will be plated into a 96 well plate and the cells will be incubated in the 37° C. incubator with 5% CO2 for 18-24 hrs. Rapologues will be serially diluted into the respective cell lines into a final volume of 100 μL per well. The cells will be incubated in the 37° C. incubator with 5% CO2 for up to six days. Cell proliferation will be measured by Cell Citer-Glo Assay (Promega) according to the manufacturer's directions. Untransduced cells and transduced cells without rapalogs will be used as controls.

TABLE 12
Sequences for Domains.
Sequences associate with a tag and/or
are associated with the
cloning process are underlined.

| SEQ ID | | |
|---|---|---|
| NO: | Description | Sequence |
| 78 | FGFR2IIIb | MVSWGRFICLVVVTMATLSLA |
| | signal | |
| | sequence | |
| 79 | FGFR2IIIb | IAIYCIGVFLIACMVVTVILC |
| | transmembrane | |
| | domain | |
| 80 | FGFR2IIIb | RMKNTTKKPDFSSQPAVHKLT |
| | Intracellular | KRIPLRRQVTVSAESSSSMNS |
| | domain | NTPLVRITTRLSSTADTPMLA |
| | | GVSEYELPEDPKWEFPRDKLT |
| | | LGKPLGEGCFGQVVMAEAVGI |
| | | DKDKPKEAVTVAVKMLKDDAT |
| | | EKDLSDLVSEMEMMKMIGKHK |
| | | NIINLLGACTQDGPLYVIVEY |
| | | ASKGNLREYLRARRPPGMEYS |
| | | YDINRVPEEQMTFKDLVSCTY |
| | | QLARGMEYLASQKCIHRDLAA |
| | | RNVLVTENNVMKIADFGLARD |
| | | INNIDYYKKTTNGRLPVKWMA |
| | | PEALFDRVYTHQSDVWSFGVL |
| | | MWEIFTLGGSPYPGIPVEELF |
| | | KLLKEGHRMDKPANCTNELYM |
| | | MMRDCWHAVPSQRPTFKQLVE |
| | | DLDRILTLTTNEEYLDLSQPL |
| | | EQYSPSYPDTRSSCSSGDDSV |
| | | FSPDPMPYEPCLPQYPHINGS |
| | | VKT |
| 81 | Linker | (GGGGS)n |
| 82 | Wild Type | ASRILWHEMWHEGLEEASRLY |
| | FRB | FGERNVKGMFEVLEPLHAMME |
| | | RGPQTLKETSFNQAYGRDLME |
| | | AQEWCRKYMKSGNVKDLTQAW |
| | | DLYYHVFRRISKTS |
| 83 | Wild Type | GLNDIFEAQKIEWHEGSGVQV |
| | FKBP | ETISPGDGRTFPKRGQTCVVH |
| | | YTGMLEDGKKFDSSRDRNKPF |
| | | KFMLGKQEVIRGWEEGVAQMS |
| | | VGQRAKLTISPDYAYGATGHP |
| | | GIIPPHATLVFDVELLKLE |

### EQUIVALENTS

The disclosures of each and every patent, patent application, and publication (including online publications, e.g., websites) cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A dimerization switch comprising:
(a) a polypeptide comprising a first switch domain comprising an FRB fragment or analog thereof having the ability to form a complex between the FRB fragment or analog thereof, a FK506 binding protein (FKBP) fragment or analog thereof and a dimerization molecule; and
(b) a polypeptide comprising a second switch domain comprising an FKBP fragment or analog thereof having the ability to form a complex between the FKBP fragment or analog thereof, a FRB fragment or analog thereof and a dimerization molecule;
wherein the dimerization switch comprises one or more of the following properties:
(i) the first switch domain comprises a one or more mutations each of which enhances formation of a complex between the first switch domain, a second switch domain, and a dimerization molecule, rapamycin, or a rapalog;
(ii) the first switch domain comprises a mutation at E2032 and at T2098;
(iii) the first switch domain comprises the mutation E2032I, and further comprises a mutation at one or a plurality of L2031, S2035, R2036, F2039, G2040, T2098, W2101, D2102, Y2105, or F2108;
(iv) the first switch domain comprises a mutation at E2032I and at T2098;
(v) the first switch domain comprises the mutation at E2032L, and further comprises a mutation at one or a plurality of L2031, S2035, R2036, F2039, G2040, T2098, W2101, D2102, Y2105, or F2108;
(vi) the first switch domain comprises a mutation at E2032L and at T2098;
(vii) the first switch domain comprises a T2098 mutation and one or a plurality of mutations at L2031, E2032, R2036, G2040, or F2108.
(viii) the first switch domain comprises a mutation at T2098L and at E2032;
(ix) the second switch domain comprises one or more mutations that enhance the formation of a complex between the first switch domain, the second switch domain, and the dimerization molecule, rapamycin, or a rapalog, wherein the one or more mutations comprise mutations at Q53, I56, W59, Y82, G89, I90, I91, K44, P45, or H87, or the one or more mutations comprise mutations at Q53, I56, W59, Y82, H87, G89, or I90; or
(x) (A) the first switch domain comprises one or more mutations that enhance the formation of a complex between the first switch domain, the second switch domain, and the dimerization molecule, rapamycin, or a rapalog; and (B) the second switch domain comprises one or more mutations that enhance the formation of a complex between the first switch domain, the second switch domain, and the dimerization molecule, rapamycin, or a rapalog.

2. The dimerization switch of claim 1, wherein the polypeptide of (a) and the polypeptide of (b) are on separate molecules, and activation of the switch results in an intermolecular association.

3. The dimerization switch of claim 1, wherein the polypeptide of (a) and the polypeptide of (b) are on the same molecule and activation of the switch results in an intramolecular association.

4.-13. (canceled)

14. The dimerization switch of claim 1, comprising property (ix) and one of properties (i), (ii), (iii), (iv), (v), (vi), (vii), and (viii).

15. The dimerization switch of claim 1, wherein the first switch domain comprises T2098L and E2032I, or T2098L and E2032L.

16. The dimerization switch of claim 15, wherein the second switch domain comprises one or more mutations at Y26, F36, D37, R42, K44, P45, F46, Q53, E54, V55, I56, W59, Y82, H87, G89, I90, I91, and F99.

17. The dimerization switch of claim 1, wherein the first switch domain differs at no more than 1, 2, 3, 4, 5, 6, 7, 8, 9, or 10 amino acid residues from the sequence of SEQ ID NO:2.

18. The dimerization switch of claim 1, wherein the first switch domain comprises 30, 35, 40, 45, 50, 55, 60, 70, 75, 80, 85 or 90 amino acids of the sequence of FRB, SEQ ID NO:2.

19. The dimerization switch of claim 1, wherein the second switch domain differs at no more than 1, 2, 3, 4, 5, 6, 7, 8, 9, or 10 amino acid residues from the sequence of SEQ ID NO:1 or 3.

20. The dimerization switch of claim 1, wherein the second switch domain comprises 30, 35, 40, 45, 50, 55, 60, 70, 75, 80, 85 or 90 amino acids of the sequence of FKBP, SEQ ID NO:1 or 3.

21.-22. (canceled)

23. The dimerization switch of claim 1, wherein:
The polypeptide of (a) further comprises an additional switch domain; and
the polypeptide of (b) further comprises an additional switch domain.

24.-29. (canceled)

30. The dimerization switch of claim 1, wherein the polypeptide comprising the first switch domain is coupled to a first moiety.

31. The dimerization switch of claim 1, wherein the polypeptide comprising the second switch domain is coupled to a second moiety.

32. The dimerization switch of claim 1, where one of the polypeptide comprising the first or second switch domain is coupled to a moiety that anchors the switch domain to a membrane.

33-34. (canceled)

35. The dimerization switch of claim 1, wherein the polypeptides comprising the first or second switch domains are, independently, coupled to a moiety from a pair of entities from Table 5.

36.-48. (canceled)

49. A polypeptide comprising an FRB fragment or analog thereof having the ability to form a complex between the FRB fragment or analog thereof, a FKBP fragment or analog thereof and a dimerization molecule, wherein the polypeptide comprises one or more of the following properties:
(i) the FRB fragment or analog thereof comprises one or more mutations each of which enhances the formation of a complex between the FRB fragment or analog thereof, a FKBP fragment or analog thereof and a dimerization molecule, rapamycin or a rapalog;
(ii) the FRB fragment or analog thereof comprises a mutation at E2032, and at T2098;
(iii) the FRB fragment or analog thereof comprises the mutation E2032I, and comprises a mutation at one or a plurality of L2031, S2035, R2036, F2039, G2040, T2098, W2101, D2102, Y2105, or F2108;
(iv) the FRB fragment or analog thereof comprises a mutation at E2032I and at T2098;
(v) the FRB fragment or analog thereof comprises the mutation at E2032L, and further comprises a mutation at one or a plurality of L2031, S2035, R2036, F2039, G2040, T2098, W2101, D2102, Y2105, or F2108;
(vi) the FRB fragment or analog thereof comprises a mutation at E2032L and at T2098;
(vii) the FRB fragment or analog thereof comprises a T2098 mutation and one or a plurality of mutations at L2031, E2032, R2036, G2040, or F2108; or
(viii) the FRB fragment or analog thereof comprises a mutation at T2098L and at E2032.

50.-57. (canceled)

58. The polypeptide of claim 49, wherein the FRB fragment or analog thereof comprises T2098L and E2032I.

59. The polypeptide of claim 49, wherein the FRB fragment or analog thereof comprises T2098L and E2032L.

60. (canceled)

61. The polypeptide of claim 49, wherein the polypeptide is coupled to a member of a pair from Table 5.

62.-68. (canceled)

69. A polypeptide comprising an FKBP fragment or analog thereof, wherein the polypeptide comprises a mutation that enhances the formation of a complex between the FKBP fragment or analog thereof, a FRB fragment or analog thereof, and a dimerization molecule, rapamycin, or a rapalog, wherein the mutation comprises one or more mutations at Q53, I56, W59, Y82, I90, I91, K44, P45, H87 or G89.

70. (canceled)

71. The polypeptide of claim 69, wherein the polypeptide is coupled to a member of a pair from Table 5.

72.-78. (canceled)

79. A nucleic acid comprising sequence that encodes:
(a) the first switch domain of claim 1;
(b) the second switch domain of claim 1; or
(a) and (b).

80.-87. (canceled)

88. A vector system comprising the nucleic acid of claim 79.

89. (canceled)

90. A cell comprising
the dimerization switch of claim 1 or a nucleic acid molecule encoding the dimerization switch of claim 1.

91.-93. (canceled)

94. A method of making a cell comprising introducing into the cell
a dimerization switch of claim 1 or a nucleic acid molecule encoding the dimerization switch of claim 1.

95. A method of activating a dimerization switch, comprising,
providing the cell of claim 90; and
contacting the cell with a dimerization molecule.

96.-97. (canceled)

98. A method of treating a subject having a disease or disorder described herein comprising administering to the subject an effective amount of a cell of claim 90.

99.-104. (canceled)

105. The method of claim 98, comprising administering a dimerization molecule to the subject.

106. The method of claim 98, comprising administering a dimerization molecule comprising an mTOR inhibitor rapamycin or a rapalog.

107. The method of claim 106, comprising administering a low, immune enhancing, dose of an allosteric mTOR inhibitor.

108.-112. (canceled)

113. A gene editing dimerization switch comprising:
(a) a polypeptide comprising a first gene editing switch domain coupled to a first moiety; and
(b) a polypeptide comprising second gene editing switch domain coupled to a second moiety;
Wherein the first or second moiety comprises a nuclear localization sequence (NLS), and wherein the other moiety comprises a gene editing protein.

114.-118. (canceled)

119. The gene editing dimerization switch of claim 113, wherein the first gene editing switch domain comprises an FRB fragment or analog thereof and the second gene editing switch domain comprises an FKBP fragment or analog thereof, further wherein:
(i) the first switch domain comprises a one or more mutations each of which enhances formation of a complex between the first switch domain, a second switch domain, and a dimerization molecule, rapamycin, or a rapalog;
(ii) the first switch domain comprises a mutation at E2032 and at T2098;
(iii) the first switch domain comprises the mutation E2032I, and further comprises a mutation at one or a plurality of L2031, S2035, R2036, F2039, G2040, T2098, W2101, D2102, Y2105, or F2108;
(iv) the first switch domain comprises a mutation at E2032I and at T2098;
(v) the first switch domain comprises the mutation at E2032L, and further comprises a mutation at one or a plurality of L2031, S2035, R2036, F2039, G2040, T2098, W2101, D2102, Y2105, or F2108;
(vi) the first switch domain comprises a mutation at E2032L and at T2098;
(vii) the first switch domain comprises a T2098 mutation and one or a plurality of mutations at L2031, E2032, R2036, G2040, or F2108.
(viii) the first switch domain comprises a mutation at T2098L and at E2032;
(ix) the second switch domain comprises one or more mutations that enhance the formation of a complex between the first switch domain, the second switch domain, and the dimerization molecule, rapamycin, or a rapalog wherein the one or more mutations comprise mutations at Q53, I56, W59, Y82, G89, I90, I91, K44, P45, or H87, or the one or more mutations comprise mutations at Q53, I56, W59, Y82, H87, G89, or I90; or
(x) (A) the first switch domain comprises one or more mutations that enhance the formation of a complex between the first switch domain, the second switch domain, and the dimerization molecule, rapamycin, or a rapalog; and (B) the second switch domain comprises one or more mutations that enhance the formation of a complex between the first switch domain, the second switch domain, and the dimerization molecule, rapamycin, or a rapalog.

120.-121. (canceled)

122. The gene editing dimerization switch of claim 113, wherein the gene editing protein is selected from the group consisting of a zinc finger nuclease; a transcription activator-like effector nuclease (TALEN); a CRISPR-associated nuclease; and a meganuclease.

123. A gene editing dimerization switch comprising:
(a) a polypeptide comprising a first gene editing switch domain coupled to a first moiety; and
(b) a polypeptide comprising second gene editing switch domain coupled to a second moiety;
Wherein the first or second moiety comprises a DNA-binding domain and the other moiety comprises a DNA-modifying domain.

124. The gene editing dimerization switch of claim 123, wherein the DNA-binding domain is a zinc finger or engineered zinc finger, a transcription activator-like effector (TALE), or a polypeptide comprising a DNA-binding domain of a Cas9.

125.-126. (canceled)

127. The gene editing dimerization switch of claim 123, wherein the DNA-modifying domain is a polypeptide having nuclease activity or a nuclease half-domain.

128.-146. (canceled)

147. The gene editing dimerization switch of claim 113, further comprising a NLS.

148. A nucleic acid comprising sequence that encodes a gene editing dimerization switch of claim 113.

149. (canceled)

150. A vector system comprising the nucleic acid of claim 148.

151. (canceled)

152. A method of modulating expression of an endogenous gene in a cell comprising administering to the cell the gene editing dimerization switch of claim 113 or a nucleic acid encoding the gene editing dimerization switch of claim 113, and contacting the cell with a gene editing dimerization molecule, such that expression of the endogenous gene is modulated.

153.-155. (canceled)

156. A method of modifying an endogenous nucleic acid sequence in a cell, comprising administering to the cell the gene editing dimerization switch of claim 113 or a nucleic acid encoding the gene editing dimerization switch of claim 113, and contacting the cell with a gene editing dimerization molecule, such that an endogenous nucleic acid sequence in a cell is modified.

157.-158. (canceled)

159. The method of claim 152, wherein the administering to the cell is performed in vivo, in vitro or ex vivo.

160.-161. (canceled)

162. A cell comprising the gene editing dimerization switch of claim 113 or a nucleic acid encoding the gene editing dimerization switch of claim 113.

163. A cell, wherein expression of one or more endogenous genes has been modulated by the method of claim 152.

164. A cell, wherein one or more endogenous nucleic acid sequences have been modified by the method of claim 156.

165.-168. (canceled)

169. A method of treating a subject having a disease associated with abberant gene expression comprising administering to the subject an effective amount of the gene editing dimerization switch of claim 113 or a nucleic acid encoding the gene editing dimerization switch of claim 113.

170. A method of treating a subject having a lysosomal storage disorder comprising administering to the subject an effective amount of the gene editing dimerization switch of claim 113 or a nucleic acid encoding the gene editing dimerization switch of claim 113.

171. The dimerization switch of claim 15, wherein the second switch domain comprises one or more mutations at Y26, F36, D37, R42, F46, Q53, E54, V55, I56, W59, Y82, H87, G89, I90, or F99.
